SEQUENCE LISTING (86 sequences)

SEQ ID NO: 1 - DNA, 931 bp, Human
tttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagcagaaaaaaaaaaaaaaaa

SEQ ID NO: 2 - DNA, 1925 bp, Human
agtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccattcctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaaccctcggccagcaggtcccctatgccaccaagggcaaccagtgggtaggatacgacgaccaggaaagcgtcaaaagcaaggtgcagtacctgaaggataggcagctggcaggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatcctacttccccttcctaattccacagctgctcaataaagtacaagagtttaacagtgtgttggcgctttgctttggtctatctttgagcgcccactagacccactggactcacctcccccatctcttctgggttccttcctctgagccttgggacccctgagcttgcagagatgaaggccgccatgtt

SEQ ID NO: 3 - DNA, 1439 bp, Human
tgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 4 - DNA, 1080 bp, Human
ggagaggactggctgggcaggggcgccgccccgcctcgggagaggcgggccgggcggggctgggagtatttgaggctcggagccaccgccccgccggcgcccgcagcacctcctcgccagcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaaaagaatctaaaccttacatgtgtaaaggtttcatgttcactgtgagtgaaaatttttacattcatcaatatccctcttgtaagtcatctacttaataaatattacagtgaattacctgtctcaatatgtcaaaaaaaaaaaaaaaaaa

SEQ ID NO: 5 - DNA, 586 bp, Human
agttaaaagggtgggagcgtccgggggcccatctctctcgggtggagtcttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaaaaaaaaaaaaaaaaaaaaaaaaaaaaaa

SEQ ID NO: 6 - DNA, 5641 bp, Human
ggagcacaaaccctattgtgaactatgcatgcgagtaatgtaggttgcccgctccttgtgagaatctaattaatgcctgattatctgaggtggaacagtttcatcctggtctgtggaaaaattgtcttccacgaaactagtccctggtgccaaaaatcttggagactgctgacctagagaacccctaccagagttcttgggaaatctgtatctgattagagataagtttttcaaacaaagcacagtgaccgtagaagggaagtttttcagctcatgaataatgtcaagaatgcagaagagcaaaagacaaggatacgtacatagagatttgatggaccgctttaagctcagcccatgtgccactcttcctctctcccctaaacacaccacagtctgtgctttgaccatgaatcaggcgatactttgctgtggggctgaagtgggggaagtccttcagttttattgtcagctctcttttccagtcatgtgtctagtggtatgggcatgagttctgaggatgacaaagatggtctgtgcttagatcttggccctgacagtagcttactgtgtgccttgagggagacacttaacctgtaagcctcaatgaactcatctgtaaaatgagggtcatagcactcatctagaagagtggtgaagttcatatgaattgagaaatagcagctgcccaataaatgatcctttctatgtcctttcccagggccatttgctgaggaaaaaaggaccttaccatggctaccagctatttcaggataggtatctttacccattcctattcctcagtgccacctcatcccccttggaattcagtaccaaacccgaacaggttgcttttcactggagtgtttcctgagctgccagatgtgcccctgagtggagatgggaatcagtgttatcttccccagggatgctgattctactccaggctggcaggagacattgtggtcctgacatggcagccaaccagatagcaatggtcttggcacccccaaagcttactgagagtaggacattctatagttgaagatgaggggagcatcttcaactgctgaaaccacaccttggcctggctgtacctgctcctatcttgaggccaaaataggagggagcacaaaagaacatggtggggtagatctgtgtgcacgtgacaccagtgaggtgctggagtgggccgtgagagtctttagtgagaatatgttggctcatgttttggaaacctttgaaaattgtcctgtggtaagggaatgagaacagtacctctctctttgtgctgttgacaggatgagttcatgtctgtcaggtgttttagaacagtgactcccatagtgctcagtaagtgtttccaagctagcagctctggctcagcaattccagtttgtacatgtcccccaaggaaatgactaagtatgtgcagaaagcattctttataacagggttagaattggaaacaacctaaatttccaacaaaaggggagattggctgattgattcatgggatggaatagtatgtgatcacgaaataagtcattttaagacaaagattttatggtaaatttattaagtgaataaacaaaggttttaaaacaatacagaatggaatatgctttttttttttttttttttttcctcctagagacaggatctccatatgttgcccaggctggtcttgtgcgccaccatgtctgggtaaagaatggatctatttttaaaaggaaactttttattttggagtaattttaagtttacagaaaggttctaaaaatagtatagagagtttctgtatatacttcacctagtttcctctaatgttaacattttacttaagtatggtacatttgtcaaaactaagagattaacattggtatattactagactgactatatttggttttcagtagtttttctactaatgtcttttctatccaggctccaaccctttataccacattgcatttttttttttttttatttaaaagtgtatgtacatagaaaagcatccgggtgcatatagatgaaaatgttaacaatgatcatagctgagtggttgaattaagggagatttttgttttaaagttcttagtatgtcttctaatttttctatgtttaacatgtcacataattttaaaacacaaaactaacaattcaggctgagttggtgccctctgattaccccttgtatggctatactgtgtatgtttagctgagtcttttcaacactcagctaaagacggcaaatgaataagtctgcctcatccctctttccctatcatgcagcccagggcccggcacagaggaggcactgataatatctagttgaataatgggtgaagtcagagaagtttattgctaccaactctcggagataaactagtcttagcactctttttcagcttaaggaaacccaggcttatgtttccccagtttagtgaccccagcaggacaggacctgtttttaaagtttgcagtctcagggctgacttttatgcccatcgctgaattagttgctgtggccaggagcatggaatatgcctcttggccaggcttgggtccactgaccaggcctttagatggcttcccaaagaaaagtcagggttctgctatccaaaggaggtaagatggatactgaggaagttaaatacaacatcagagccatcccctaaacacaggcattttaatctctaattccttttgccatactatatgtcatgcttgggctccctgatgagaatagtaccacattaatgctattcatcattaaatcctagcatccagcacagtgcttgtatgtagcagggacctagtcaacatacaagtgaataaatacatttcttggtgacctaaacaaaatcatttaactagatgtttaaatcagaatgctttgagttgtaaataacagaaaactggctcaataaaggacatttgttgactcatgcaactgaaaagtctagtggagcccagcagtcaggcatggtgtggtcaggacttcacttatgtgccagtctctgactgcccccttggtatcatcaggcttgtctctctcatggtagcaaaatgactgcagcagttccagacgtgacatctgtactccgcaacatccagaacaagagaaaatacagagaccctgtatcacagaattcccagcaaaagtcctgagagtcactgtgaactgaataagattacatgcccacctctgaatcagcaattagtggcaagggttgtttactggcttagcctagatcatgtacctcacccttagaactggggagataggggagggagagagtagaatcagcttccctgaaactacatggattcctgatgggaactgagactttggggaaggggaaagcagtccggatcctgaggatgtaaccaataggaatccagctcaattgaataccctgaattgtgtggcataaacaagggctgcaggagttagagggcaatgagtgaaggttgcatcagtcagagcagggaggcttcatggaggagaatgagagactggagccaaaccctgaagagataaaagatttgggcaatggggtagggaggtggaaaggaaggaacattgcccatgtgctgtgccagacatcatactaaaccctctgtatgccgggcgtggtggctcacgcctgtaatcccaacactttgggaggctgagacaggtggatcacctgaggtcaggagttcgagacaagattggccaacatggagaaaccccgtctctacaaaaatacaaaaattagtcaggcatggtggtgcactcctttagtcccagctactcaggagactgaggtgggggcattgcctgaacccgggaggcagaggttgcggtgagccgagattgtgcctctgcactgcagcctgggtgataaagcaaaactccatctcaaaaaaaaaaaaaaaattctctgtatgcattatctcagttaacccttacaatgacctgcaagtttggtatcatcattatccccattttacagttgagaaactgaaacccaggggtcacacagtaagtgttgaggttgagacttggacccagcttgtctggctgagaagcttgtgcctggagctatgacgctggtgagtaaagtagacagtggtcacctgggaagtttgcattagtgtgaagtaagtggcctgacctcaggtcatcacagaggtagtgcagaatgtgaagttcttccctcagctgtgctgtttttggggcagcggggatcagaaccaaagcacaattcctttgaaagtttgccaaactttgaccttaaccatgtgaaaccttgtgcctttcctgctcaccagatggattcttcagacaaggcctaggtgctggttctgctccttactaggtggccttgaatgtatcattcacagtctttgagcctcagtttccctatttgcaaaatagacacagtaataccacatttattgattgaacagatttactcttgccttatctttgctgggcctgggagaccaagaaacaaagatgaatgagacagtgcctgcccccaaggacctcccagtctagcagaggagatggatataaaatcagagagacttatggagagcagtggaaaacatagatcagggagcgatcaacttagcttaagtggatctgggaaggcctcaaagtaggggacatctgagccacaccttgaatggttagtaagagtttgccaggcagccacttggggcagagttttgtgagaatcagataagctgtgtcaaagtaaatgttttataaattgggctgcctttcccttctaggttcatacctcagcttgcattgctgctgtaggaaacataagaaatgccctagaaacccctccctgggctgagagttcctagaattaggggccggggaagagttttgaatgcatgctaaagtctaggctcatcatcttgccacacaaggcctgtttggcttcatctttctcttaattttatgctcacgcaaaacaaccacctagagttccatgaaatcattacctccctgtttctcctcctatgtcttcttcttctccacacccacctggcaacctctaatttgtttcctggaaatgtgattgacccacactccactcacgcagcctcagcacccttttatttgtaaatacatggactcatcttctccaggggactggggctccttcaaagacaaagagtaggtctcaatctctaccctgccacacctagcaccaactgatcagagctgcatctctgatgcagggataggtagttgggagtctcacttcttccacaaacattgagcttctgccacaggcctgctggcgtcaagtcaggcaaaaaggattcaggaagcaaagggtacaatccaaccctggaagcagtcacagtccagagagaaactcacatgtaaacagtccattgtaatactgggtgatgtgtgctgggatgaaaggatatctggaacatgaaggggacacaggagagatcaactactttgggcacacgggaaattgagcaggaatctgctgggtggtgaaagacagacactagagcttgtacgaggcatggaagcatgaaaaagcacagcaggttccagggtcagtgagtatgcgtatggctgaaggaacatgggactagttctagatcgcga

SEQ ID NO: 7 - DNA, 2764 bp, Human
agctctcagagggaattgagcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatagaaggggtctggcccgggcatccacgtagggggcaggggcaggggcgggcggagggaggggaggggtgaaatccatactgtagacactctgacaagctggccaaagtcacttccccaagatctgccagacctgcatggtcaagcctcttatgggggtgtttctatctctttctttctctttctgtttcctggcctcagagcttcctggggaccaagatttaccaattcacccactcccttgaagttgtggaggaggtgaaagaaagggacccacctgctagtcgcccctcagagcatgatgggaggtgtgccagaaagtcccccctcgccccaaagttgctcaccgactcacctgcgcaagtgcctgggattctaccgtaattgctttgtgcctttgggcacggccctccttcttttcctaacatgcaccttgctcccaatggtgcttggagggggaagagatcccaggaggtgcagtggagggggcaagctttgctccttcagttctgcttgctcccaagcccctgacccgctcggacttactgcctgaccttggaatcgtccctatatcagggcctgagtgacctccttctgcaaagtggcagggaccggcagagctctacaggcctgcagcccctaagtgcaaacacagcatgggtccagaagacgtggtctagaccagggctgctctttccacttgccctgtgttctttccccaggggcatgactgtcgccacacgcctctgtgtacatgtgtgcagagcagacaggctacaaagcagagatcgacagacagccaggtagttggaactttctgttccctatggagaggcttccctacacagggcctgctattgcagaatgaagccatttagagggtgaaggagaaatacccatgttacttctctgagttttagttggtctttccatctatcactgcattatcttgctcattcttcagttctctactccctcttgtcagtgtagacacaggtcaccattatgctggtgtatgtttatcaaagagcacttgagctgtctgaagcccaaagcctgaggacagaaagaccctgatgcaggtcagcccatggaggcagatgcccttgctgggcctgggggttttccaagccctcagctggtcctgaccaggatggagcaagctcttcccttgctcatgagctcctgatcagaggcatttgagcagctgaataacctgcacaggcttgctgtatgacccctggccacagccttccctctgcattgacctggaggggagaggtcagccttgacctaatgaggtagctatagttgcagcccaaggacagttcagagatcaggatcagctttgaaggctggattctatctacataagtcctttcaattccaccagggccagagcagctccaccactgtgcacttagccatgatggcaacagaaaccaagagacacaattacgcaggtatttagaagcagagggacaaccagaaggcccttaactatcaccagtgcatcacatctgcacactctcttctccattccctagcaggaacttctagctcatttaacagataaagaaactgaggcccacggtttcagctagacaatgatttggccaggcctagtaaccaaggccctgtctctggctactccctggaccacgaggctgattcctctcatttccagcttctcagtttctgcctgggcaatggccaggggccaggagtggggagagttgtgatggaggggagaggggtcacacccaccccctgcctggttctaggctgctgcacaccaaggccctgcatctgtctgctctgcatatatgtctctttggagttggaatttcattatatgttaagaaaataaaggaaaatgacttgtaaggtcaaaaaaaaaa

SEQ ID NO: 8 - DNA, 3327 bp, Human
ggaagaggaggggggagaaggaggagctgaagcgggaagagcggcaggcagcggctcggcggagttgcagcagaggcggcggcgacgctgagacaccgcagcttccctgagcgccgagtccctccggggacagcagcagggagcgcccgcgcagccaccgagcctctgcccagccaagccgccgtcgccgcgccgggggaccgccagccatggccgcgccgggggatccgcaggacgagctgctgccgctggccggccccgggtcccagtggctcaggcaccggggggagggggagaacgaagcggtgacgccgaaaggggccacgccggcgccgcaggctggggagcccagcccggggttgggcgccagggcccgggaagcggcgtcgcgggaagccggctcgggccccgcccggcagtcgcccgttgccatggaaactgcatccacaggtgtggcaggtgtttccagtgccatggaccacaccttctcaacaacatcaaaagatggggaaggatcgtgttacacatctctcatttctgacatctgctatccacctcaggaggattctacatattttactggaattcttcagaaggaaaatggccacgtcaccatttcagagagccctgaggagctgggtacacccggcccctccttaccagatgtgcctgggatagagtctcgtggcttatttagttctgattctggaatagagatgactcctgcagagtccacggaagtgaacaagatcttagcagaccctctggaccagatgaaagcagaggcctataaatacattgacataaccagacccgaggaggtgaagcaccaagaacaacatcaccccgagctggaagataaagacttggactttaagaataaagacactgacatctcaattaaacctgaaggagtccgtgaacctgacaaaccagctcctgtggagggaaaaatcatcaaggaccatttattggaagaatccacatttgctccatacatagatgatctctctgaagaacagcgcagggctcctcagatcaccacccctgtcaaaatcacactgacggaaatagaaccttctgttgaaaccactacccaagagaagacccctgagaagcaagatatatgtctaaagccaagtcctgacacagtccccactgtcactgtctcggagcctgaagacgacagcccaggatctatcacccctccatcttctggaacagaaccatctgctgcagaatcccaggggaaaggcagcatctccgaggatgagctgatcaccgccatcaaagaagcaaagggattatcgtatgaaaccgccgagaacccacggccggtgggccagctggccgacaggcccgaggtcaaggccaggtccggaccgccaaccatccccagccccctggaccacgaggccagcagcgcggagtcgggggactcagagatcgagctggtgtccgaggaccccatggccgcggaggacgcgctgccctcaggctatgtgagctttggccacgtgggcggcccgccgccctcgcccgcctcgccatccatccagtacagcatcctgagggaggagcgcgaggccgagctggacagcgagctcatcatcgagtcgtgcgacgcctcctcggcctcggaggagagccccaagcgggagcaggactcacccccgatgaagcccagcgccctggatgccatccgggaggagactggcgtccgggccgaggagcgtgcgccaagccggcggggcctggccgagccgggttccttcctcgactacccctcaactgagccccagcctggccccgagctgccccctggagacggagccctggagcctgagacgcccatgttgccacggaagcctgaagaagactcgagttccaaccaaagtcctgcggccacaaagggccctgggcctctaggtcctggcgccccgcccccactgctgtttctcaataagcaaaaagctattgacctgttgtattggcgggacatcaagcagacgggcatcgtgtttgggagtttcctgctgctgctcttctccctgacccagttcagcgtggtgagcgtcgtggcctacctggccctggccgcactctcagccaccatcagtttccgcatctacaagtctgttttacaagcagtgcagaaaaccgacgaaggccaccctttcaaggcctacttggagcttgagatcaccctttctcaggagcagattcagaagtacacggactgcctgcagttctacgtgaacagcacacttaaggaactgaggaggctcttccttgtccaggacctggtggattccttaaaatttgcagtcctgatgtggctcctgacctacgttggcgctctcttcaatggcctgaccctgctgctcatggctgtggtttcaatgtttactctacctgtagtgtatgttaagcaccaggcacagattgaccaatatctgggacttgtgaggactcacataaatgctgttgtggcaaagattcaggctaaaatcccaggcgctaagaggcacgctgagtaaactgatttcccaccggggactggacacaaacaggaatgtctggagtggtaacagctctcttcttactcattactgcaaattgattgtctttcccccctccctccagtaccataatcttagagacaaaccttaaaacagctgtttttaggctgttccttgtactcttaggatatttgagtcacttgtgtcaaccactaaagtatagagaaaagtgtattagatgtggtttttaattttgtgttgctaaaaaaagtgcatgatggtgagagcccaagttatctttccctcttcggtgttcttcttctcttctctgcaatgcttctgtagcttctaatgttccccgtggctaggcctttcctgccgagtgctctgatgcaatagtggaaatcgcttatatgtccttgggttgctggttggattaatctttaataacaatatatagaattgtagactgatgttttagcatttttccaacacacacaacgtaaaaataaaagcagtcgaccgcacttatggtaatcagttttgtataacttaaaataattaaataaatgaataaatccaaaacaaacatgcagtacttttgttgtatgggattggtgggctgatttacatgtatggttactaaaaagtaccagcatgttaactttattacaatttgtattactttctctgtagttcctaatggattcaattacggactctggatatttgcacttatgtacttgatactgaatgcataaataaatgttactaaatgtagaatgttaaaaaaaaaaaaaaaaaa

SEQ ID NO: 9 - DNA, 1661 bp, Human
tgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtg

SEQ ID NO: 10 - DNA, 5644 bp, Human
aggagagagggagaagagagcgcgagagagggtgagtgtgtgtgagtgcatgggagggtgctgaatattccgagacactgggaccacagcggcagctccgctgaaaactgcattcagccagtcctccggacttctggagcggggacagggcgcagggcatcagcagccaccagcaggacctgggaaatagggattcttctgcctccacttcaggttttagcagcttggtgctaaattgctgtctcaaaatgcagaggatctaatttgcagaggaaaacagccaaagaaggaagaggaggaaaaggaaaaaaaaaggggtatattgtggatgctctacttttcttggaaatgcaaaagattatgcatatttctgtcctcctttctcctgttttatggggactgatttttggtgtctcttctaacagcatacagataggggggctatttcctaggggcgccgatcaagaatacagtgcatttcgagtagggatggttcagttttccacttcggagttcagactgacaccccacatcgacaatttggaggtggcaaacagcttcgcagtcactaatgctttctgctcccagttttcgagaggagtctatgctatttttggattttatgacaagaagtctgtaaataccatcacatcattttgcggaacactccacgtctccttcatcactcccagcttcccaacagatggcacacatccatttgtcattcagatgagacccgacctcaaaggagctctccttagcttgattgaatactatcaatgggacaagtttgcatacctctatgacagtgacagaggcttatcaacactgcaagctgtgctggattctgctgctgaaaagaaatggcaagtgactgctatcaatgtgggaaacattaacaatgacaagaaagatgagatgtaccgatcactttttcaagatctggagttaaaaaaggaacggcgtgtaattctggactgtgaaagggataaagtaaacgacattgtagaccaggttattaccattggaaaacatgttaaagggtaccactacatcattgcaaatctgggatttactgatggagacctattaaaaatccagtttggaggtgcaaatgtctctggatttcagatagtggactatgatgattcgttggtatctaaatttatagaaagatggtcaacactggaagaaaaagaataccctggagctcacacaacaacaattaagtatacttctgctctgacctatgatgccgttcaagtgatgactgaagccttccgcaacctaaggaagcaaagaattgaaatctcccgaagggggaatgcaggagactgtctggcaaacccagcagtgccctggggacaaggtgtagaaatagaaagggccctcaaacaggttcaggttgaaggtctctcaggaaatataaagtttgaccagaatggaaaaagaataaactatacaattaacatcatggagctcaaaactaatgggccccggaagattggctactggagtgaagtggacaaaatggttgttacccttactgagctcccttctggaaatgacacctctgggcttgagaataagactgttgttgtcaccacaattttggaatctccgtatgttatgatgaagaaaaatcatgaaatgcttgaaggcaatgagcgctatgagggctactgtgttgacctggctgcagaaatcgccaaacattgtgggttcaagtacaagttgacaattgttggtgatggcaagtatggggccagggatgcagacacgaaaatttggaatgggatggttggagaacttgtatatgggaaagctgatattgcaattgctccattaactattacccttgtgagagaagaggtgattgacttctcaaagcccttcatgagcctcgggatatctatcatgatcaagaagcctcagaagtccaaaccaggagtgttttcctttcttgatcctttagcctatgagatctggatgtgcattgtttttgcctacattggggtcagtgtagttttattcctggtcagcagatttagcccctacgagtggcacactgaggagtttgaagatggaagagaaacacaaagtagtgaatcaactaatgaatttgggatttttaatagtctctggttttccttgggtgcctttatgcggcaaggatgcgatatttcgccaagatccctctctgggcgcattgttggaggtgtgtggtggttctttaccctgatcataatctcctcctacacggctaacttagctgccttcctgactgtagagaggatggtgtctcccatcgaaagtgctgaggatctttctaagcaaacagaaattgcttatggaacattagactctggctccactaaagagtttttcaggagatctaaaattgcagtgtttgataaaatgtggacctacatgcggagtgcggagccctctgtgtttgtgaggactacggccgaaggggtggctagagtgcggaagtccaaagggaaatatgcctacttgttggagtccacgatgaacgagtacattgagcaaaggaagccttgcgacaccatgaaagttggtggaaacctggattccaaaggctatggcatcgcaacacctaaaggatcctcattaagaaccccagtaaatcttgcagtattgaaactcagtgagcaaggcgtcttagacaagctgaaaaacaaatggtggtacgataaaggtgaatgtggagccaaggactctggaagtaaggaaaagaccagtgccctcagtctgagcaacgttgctggagtattctacatccttgtcgggggccttggtttggcaatgctggtggctttgattgagttctgttacaagtcaagggccgaggcgaaacgaatgaaggtggcaaagaatgcacagaatattaacccatcttcctcgcagaattcacagaattttgcaacttataaggaaggttacaacgtatatggcatcgaaagtgttaaaatttaggggatgaccttgaatgatgccatgaggaacaaggcaaggctgtcaattacaggaagtactggagaaaatggacgtgttatgactccagaatttcccaaagcagtgcatgctgtcccttacgtgagtcctggcatgggaatgaatgtcagtgtgactgatctctcgtgattgataagaaccttttgagtgccttacacaatggttttcttgtgtgtttattgtcaaagtggtgagaggcatccagtatcttgaagacttttctttcagccaagaattcttaaatatgtggagttcatcttgaattgtaaggaatgattaattaaaacacaacatctttttctactcgagttacagacaaagcgtggtggacatgcacagctaacatggaagtactataatttacctgaagtctttgtacagacaacaaacctgtttctgcagccactattgttagtctcttgattcataatgacttaagcacacttgacatcaactgcatcaagatgtgacatgttttataaaaaaaggaaaaaaaacatttaaaactaaaaaatatttttaggtattttcacaaacaaactggcttttaaataaatttgcttccatattggttgaataagacaaaaacaattaaactgagtgggaagtgaataaaaaaaggctttaggtatcgattccatatttttcaaagccaaatatgtaaatgctaaggaaagtaaacaaagaggagattccaatcttgtaatttaatattgttattaaaactttaatgtatcctattctttaacatttggtgttaatataaaattacttggcaatgcttgacatttgaaataaacatttttctattgttttattgcaagtggtccaattaattttgcttagctacagtttggtcataaatcaagtgagtttaaagacactaccaagttgttaggtgcccagagaaaatttctcccttttaaaaaggccaggtgatttttcaaatgtaatcttgcccccaaagtaatatctgaatatctttttgacatgtctaaatatatatatatataaagaaatatttgttaacacaaaagcatttgatctatgtagataaatgctaatagatttaaaaagctaatattaacaaataccagaatacgtgaagttccatttttaaagtgtttgagcttacagaagagaaacattcattttaaatgaagtaaaaaatgccttgaaagtaattctttagatagttgcccattgattaaattccaaaaactaaatatgtttttagctttaaaattataaaagctgtcataaactttatatattatgaattttaaaatatgtttgagtctcctgcaatatagtttcatcccattgacatcaattaaaaataaccctaatatattatttttatatttattcctcaggtggaatggctattttaatatgcccagtgtggataaaatgtcacatttctgtaacttttgactaaagagcctatatttatctagttaatgaatttaaaggatctatctttcccttcataaaatacctcttatttccattaaagccccccaagtttaattaatttaggattttgaatgattattgacatccaatagttatttttaatatttgtattcttgttatttctggaagaaagcctttgtgtagcacttggtattttgcaaagtgcttttaaaacattcttacttaccgtatttcatagaagggaaggaaaaatgtaaggtttaacagtaagcacttgcattgaacatggaggcatgtggtatcatgatattcttcactaaatttagctgtccctaatcacagatcctaaggtaatataatataattttagtgcatttctcctcatcaggaatgctggaggtgcattttaagttttaataataagtgctagaatgaccaaattgcagactaattgtttccatattgtacttaaaatgagtttttaaaagtgaaaaagaaatgactatatacaatcaatgctatttattgtacctctgggcctactcttctaaaaattgtagcttatcgatttttctctgtcaagcttgaactaatgtaaataattgaaataatgtaaagttatattttcatgtttttatagatacaacatgacaagaatacataatgtaagagtatttcaactatggataatgttgattggataatgcacatctcagttacaagcagtactcatagtttaatatccatgtaacggtgcatcaatatattgctatataaatatgtctgtgtgcatataagtgaaaagtggtcaaacaagagtgatgacagctgtctaaaggtttttttattcattttatataaaaactgttatggaaagaccaaaatgtttatgaactattcttatgtaaatttacaattgtcctttactgtacttttttgtttacagtatagtaccttattttctgctgtgttaagtgggtgtcaaactccaagaagacatacactttctataacttctattgaagatattggaatttccaatttttcatgtgtactatgtcagaaaatgctttcgattttatttttaaatctaacatcggatggcttttccggagtgttgtaaaaacttcaatcatacataaaacatgttcttacaaaaggcaaa

SEQ ID NO: 11 - DNA, 817 bp, Human
gaaggaggcccagacagtgagggcaggagggagagaagagacgcagaaggagagcgagcgagagagaaagggttctggattggaggggagagcaagggagggaggaaggcggtgagagaggcgggggcctcgggagggtgaaaggagggaggagaagggcggggcacggaggcccgagcgagggacaagactccgactccagctctgacttttttcgcggctctcggcttccactgcagccatgtcactcctcttgctggtggtctcagcccttcacatcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcatggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtgagcgccccgcctcgctcggctgcccccgccccttcccggcccccctcgccgcgcgtcctccaaaaaaataaaactttaacggcgg

SEQ ID NO: 12 - DNA, 662 bp, Human
accgccgacgcagacccctctctgcacgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgaccaagctcagttcaatgagcaaatctccatactgtttctttcttttttttttcattactgtgttcaattatctttatcataaacattttacatgcagctatttcaaagtgtgttggattaattaggatcatccctttggttaataaataaatgtgtttgtgct

SEQ ID NO: 13 - DNA, 4445 bp, Human
cgctccccgctcccgtggctgccgccgccccggggaagaagagacaggggtggggtttgggggaagcgagagaggaggggagagaccctggccaggctggagcctggattcgaggggaggagggacgggaggaggagaaaggtggaggagaagggaggggggagcggggaggagcggccgggcctggggccttgaggcccggggagagccggggagccgggcccgcgcgccgagatgttgctgctgctgttactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaa

SEQ ID NO: 14 - DNA, 7560 bp, Human
accggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgaatccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtagatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtgaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtttggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcggctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacttaacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacggcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctcaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattggagggaccactgggtgagagaggaataaggcggcccagagcgaggaaaggattttaccaaagcatcaatacaaccagcccaaccatcggtccacacctgggcatttggtgagaatcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgaaaaaaaaccggaattc

SEQ ID NO: 15 - DNA, 5411 bp, Human
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 16 - DNA, 2505 bp, Human
gtgcggatgcttattatagatcgacgcgacaccagcgcccggtgccaggttctcccctgaggcttttcggagcgagctcctcaaatcgcatccagattttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagcaaaaaaaaaaaaaaaa

SEQ ID NO: 17 - DNA, 3665 bp, Human
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 18 - DNA, 2566 bp, Human
cgaggcgctggtgcacgggggcagcgcgcagcaggccggcgggcaggcgggcgggctggctggcaggcaggactgggatcgaggcccagaaaacggagcagcgggcaccagggaggcctggaacggggcgagcgccatgagcaacaaatgcgacgtggtcgtggtggggggcggcatctcaggtatggcagcagccaaacttctgcatgactctggactgaatgtggttgttctggaagcccgggaccgtgtgggaggcaggacttacactcttaggaaccaaaaggttaaatatgtggaccttggaggatcctatgttggaccaacccagaatcgtatcttgagattagccaaggagctaggattggagacctacaaagtgaatgaggttgagcgtctgatccaccatgtaaagggcaaatcataccccttcagggggccattcccacctgtatggaatccaattacctacttagatcataacaacttttggaggacaatggatgacatggggcgagagattccgagtgatgccccatggaaggctccccttgcagaagagtgggacaacatgacaatgaaggagctactggacaagctctgctggactgaatctgcaaagcagcttgccactctctttgtgaacctgtgtgtcactgcagagacccatgaggtctctgctctctggttcctgtggtatgtgaagcagtgtggaggcacaacaagaatcatctcgacaacaaatggaggacaggagaggaaatttgtgggcggatctggtcaagtgagtgagcggataatggacctccttggagaccgagtgaagctggagaggcctgtgatctacattgaccagacaagagaaaatgtccttgtggagaccctaaaccatgagatgtatgaggctaaatatgtgattagtgctattcctcctactctgggcatgaagattcacttcaatccccctctgccaatgatgagaaaccagatgatcactcgtgtgcctttgggttcagtcatcaagtgtatagtttattataaagagcctttctggaggaaaaaggattactgtggaaccatgattattgatggagaagaagctccagttgcctacacgttggatgataccaaacctgaaggcaactatgctgccataatgggatttatcctggcccacaaagccagaaaactggcacgtcttaccaaagaggaaaggttgaagaaactttgtgaactctatgccaaggttctgggttccctagaagctctggagccagtgcattatgaagaaaagaactggtgtgaggagcagtactctgggggctgctacacaacttatttcccccctgggatcctgactcaatatggaagggttctacgccagccagtggacaggatttactttgcaggcaccgagactgccacacactggagcggctacatggagggggctgtagaggccggggagagagcagcccgagagatcctgcatgccatggggaagattccagaggatgaaatctggcagtcagaaccagagtctgtggatgtccctgcacagcccatcaccaccacctttttggagagacatttgccctccgtgccaggcctgctcaggctgattggattgaccaccatcttttcagcaacggctcttggcttcctggcccacaaaagggggctacttgtgagagtctaaagagagagggtgtctgtaatcacactctcttcttactgtatttgggatatgagtttggggaaagagttgcagtaaagttccatgaagacaaatagtgtggagtgaggcggggagcatgaagataaatccaactctgactgtaaaatacatggtatctctttctccgttgtggcccctgcttagtgtcccttacctggcttagcgttctgtttcaccagtttccaagtttattgccctcaaaatctttagaatagttaaattggcttgtttaaggttcttgctgccccacaacacaccttgcccatgcacaaggaatgaattttttcctaccattatggctttgtgcttgttcttcctcttacctgtaatagcctcaccttccctagttctttgcattcgtccttagaatactgtattgttacagctgaaagacagtaaagaccatttagtcctcaccttctgttttagagttgagcaaactgaagcccacagaggtggaacttaattacctaagagccacaataagccactggtatctgggggactagaacacaaatccaacgcttttcccacctctttggatgttttccccaattatcctccttcactccctgtcatagttaccgatggtgtcccgttgtgtgggtttactctgtgctaagttgtcttacacttctcaaatgctactcagtatatagccttaagtcttactgttttgtgcggtgtgtctccagctgattttaacttttttgatggtagaaattttatctcttcttccttttgtatcctccattgtatcttcatacaaaggacagtacacacttgggtaattaaaaataaaagttgattgaccataaaaaaaaaaaa

SEQ ID NO: 19 - DNA, 8449 bp, Human
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 20 - DNA, 1629 bp, Human
attcatgaaaatccactactccagacagacggctttggaatccaccagctacatccagctccctgaggcagagttgagaatggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgtagctctcacatgcacaggggcccatggactcttcagtctggagggtcctgggcctcctgacagcaataaataatttcgttggaaaaaaaaaaaaaaaaaaaaaaaaaaaaaaaaaaaaaaaa

SEQ ID NO: 21 - DNA, 2652 bp, Human
gacaatgtgcaaatgacaccgttttgtgctcaccagggcaaagcaagggagcgccctcacttcagcatctcagccctgctaaagaaaaagctgctgggtaacatcctttgtttttgcccagggaagctttagctgtgattcccttcagccggctcctgaatgtcaaagccagcacaggccagccagaagatgacactgagactacaggtttggaaggcggcgttgccatgccaggtgccgaagatgatgtggtgactccaggaaccagcgaagaccgctataagtctggcttgacaactctggtggcaacaagtgtcaacagtgtaacaggcattcgcatcgaggatctgccaacttcagaaagcacagtccacgcgcaagaacaaagtccaagcgccacagcctcaaacgtggccaccagtcactccacggagaaagtggatggagacacacagacaacagttgagaaagatggtttgtcaacagtgaccctggttggaatcatagttggggtcttactagccatcggcttcattggtgcaatcatcgttgtggttatgcgaaaaatgtcgggaaggtactcgccctaaagagctgaagggttacgccctgctgccaacgtgcttaaaaaaagaccgtttctgactctgtgccctgtccctgagctcgtgggagaagatgacccgtggaacacttgcctggcccactcagaatccacggtgacctctccgcttgccaaaataaccgaaggaaagaccgttcaccagacttggctcctctaaacatttgctgttcaaacatgtttttgaatatacattctataaaagattatttgaaagacaaaattcatagaaaatggagcaaaactgtataaactgatttgtaactaacactggaccattggatcgatattatatgctgtaaccatgtgtctccgtctgaccattcttgttattgttaaaatgcagaggaatctggaaatatttatatccacggagtccttggatccagtgctacgtcagtaaatagcaccagcattttgcaattgctgatctgctgaaatgtacacattctggtctagtttggtctatcttttaaagcctgatctggtgtgaataatcaactaggaaatctaaacttggataacacgtggtgaacaactgcctttagctggtccagattaatcatttcaaagacatccattttagatcacaagcaggaagtcgatagtctcaaaggcactttgtttctcccaagtaggccaccaggcagcctctagagttgctttacccaaatccttctccagccatgacttggtgactctaagcttgctcccacctgccccctccacttccctcagatgatgaggagccagggctaagggggcagccttctctcttcccagtgatgcacatccttcacattggctgctttgttctggaatatggatatctcagcctggatgccgaggaagctgctggatgcttaatggtgctagaggctcaagtgtgtttgaaaccaagagccagttgtcccccatgcagaaagaaatcctgtgtgagcctctggtatgagaaataaaatctgccagttttataacattcactttctgcctctgaggaaagatacagggaacaaaaatcaatttgtacagtcttaatattaaaagcagcttgactaaatacctgatttaaaaatagaagacatccccagtcctcatgacataccgcaaatatctgtggggtcctgttgaaaagaacaaaataaaggagcccaaggggtcattctgtctcagcaccatccagcctggcacttctcttcccatatatccattggatttttttttttttttcctaaacaaagtttttacactgagcagatgctctgtcatgatggcggttgtgcaattctggtatcctctaaatttgtaagcattcataaaacaggaaaaagtaaactatcattcggaagcacagcccattcctcccattttttgcaatgatgtctggatgttattttaaacagtgtgtctgtgtgttcccaaatccagctggccccaccagctcagattccattttttttgtgtgtgtgtgtgaaacgtagtctgcaactctgcctcccggcaattatacatgtgtcaggatgtcaaaaagcaattctcctgcctcagcctcctgagtagctgggactacaggttcctaccaccacacccggccaatttttgtatttttagtagagatggggtttcaccgtatcggcgaggatgatctctatctcttgacctcgtgatctgcccgcctcggcctcccaaagtgctgggattacaggcgtgtgccactgcgctcggcctcagattccatatttgaacaccagctgattgagagaaggggaatgagaagagctggatgagtttaaataactcattgttcagattcctgaacaggagttgggataatggccatcttttctttcctatcctttcttcccccctcactgtgaaaaataacagtccaccccaagtcatacactggacccagtgcctgcggggacaggactgtgggtttcttggtcacacctgtgttggtgctcaatgcagtgtagacatgttttcaaataaaacaaatgattgtgtacaaaaaaaaaaaaaaaaaa

SEQ ID NO: 22 - DNA, 1574 bp, Human
tcaccacggcggcagccctttaaacccctcacccagccagcgccccatcctgtctgtccgaacccagacacaagtcttcactccttcctgcgagccctgaggaagccttgtgagtgcattggctggggcttggagggaagttgggctggagctggacaggagcagtgggtgcatttcaggcaggctctcctgaggtcccaggcgccagctccagctccctggctagggaaacccaccctctcagtcagcatgggggcccaagctccaggcagggtgggctggatcactagcgtcctggatctctctcagactgggcagccccgggctcattgaaatgccccggatgacttggctagtgcagaggaattgatggaaaccaccggggtgagagggaggctccccatctcagccagccacatccacaaggtgtgtgtaagggtgcaggcgccggccggttaggccaaggctctactgtctgttgcccctccaggagaacttccaaggagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaaaaaaaaaaaaaaaaaa

SEQ ID NO: 23 - DNA, 1579 bp, Human
gaggaggtgcttgccagacactgggtcatggcagtggtcggtgaagctgcagttgcctagggcagggatggagagagagtctgggcatgaggagagggtctcgggatgtttggctggactagattttacagaaagccttatccaggcttttaaaattactctttccagacttcatctgagactccttcttcagccaacattccttagccctgaatacatttcctatcctcatctttcccttcttttttttcctttcttttacatgtttaaatttaaaccattcttcgtgaccccttttcttgggagattcatggcaagaacgagaagaatgatggtgcttgttaggggatgtcctgtctctctgaactttggggtcctatgcattaaataattttcctgacgagctcaagtgctccctctggtctacaatccctggcggctggccttcatcccttgggcaagcattgcatacagctcatggccctccctctaccataccctccacccccgttcgcctaagctcccttctccgggaatttcatcatttcctagaacagccagaacatttgtggtctatttctctgttagtgtttaaccaaccatctgttctaaaagaagggctgaactgatggaaggaatgctgttagcctgagactcaggaagacaacttctgcagggtcactccctggcttctggaggaaagagaaggagggcagtgctccagtggtacagaagtgagacataatggaatcaggcttcacctccaaggacacctatctaagccattttaaccctcgggattacctagaaaaatattacaagtttggttctaggcactctgcagaaagccagattcttaagcaccttctgaaaaatcttttcaagatattctgcctagacggtgtgaagggagacctgctgattgacatcggctctggccccactatctatcagctcctctctgcttgtgaatcctttaaggagatcgtcgtcactgactactcagaccagaacctgcaggagctggagaagtggctgaagaaagagccagaggcctttgactggtccccagtggtgacctatgtgtgtgatcttgaagggaacagagtcaagggtccagagaaggaggagaagttgagacaggcggtcaagcaggtgctgaagtgtgatgtgactcagagccagccactgggggccgtccccttacccccggctgactgcgtgctcagcacactgtgtctggatgccgcctgcccagacctccccacctactgcagggcgctcaggaacctcggcagcctactgaagccagggggcttcctggtgatcatggatgcgctcaagagcagctactacatgattggtgagcagaagttctccagcctccccctgggccgggaggcagtagaggctgctgtgaaagaggctggctacacaatcgaatggtttgaggtgatctcgcaaagttattcttccaccatggccaacaacgaaggacttttctccctggtggcgaggaagctgagcagacccctgtgatgcctgtgacctcaattaaagcaattcctttgacctgtca

SEQ ID NO: 24 - DNA, 1265 bp, Human
gttcagggcggggccggtcggtgagtcagcggctctctgatccagcccgggagaggaccgagctggaggagctgggtgtggggtgcgttgggctggtggggaggcctagtttgggtgcaagtaggtctgattgagcttgtgttgtgctgaagggacagccctgggtctaggggagagagtccctgagtgtgagacccgccttccccggtcccagcccctcccagttcccccagggacggccacttcctggtccccgacgcaaccatggctgaagaacaaccgcaggtcgaattgttcgtgaaggctggcagtgatggggccaagattgggaactgcccattctcccagagactgttcatggtactgtggctcaagggagtcaccttcaatgttaccaccgttgacaccaaaaggcggaccgagacagtgcagaagctgtgcccaggggggcagctcccattcctgctgtatggcactgaagtgcacacagacaccaacaagattgaggaatttctggaggcagtgctgtgccctcccaggtaccccaagctggcagctctgaaccctgagtccaacacagctgggctggacatatttgccaaattttctgcctacatcaagaattcaaacccagcactcaatgacaatctggagaagggactcctgaaagccctgaaggttttagacaattacttaacatcccccctcccagaagaagtggatgaaaccagtgctgaagatgaaggtgtctctcagaggaagtttttggatggcaacgagctcaccctggctgactgcaacctgttgccaaagttacacatagtacaggtggtgtgtaagaagtaccggggattcaccatccccgaggccttccggggagtgcatcggtacttgagcaatgcctacgcccgggaagaattcgcttccacctgtccagatgatgaggagatcgagctcgcctatgagcaagtggcaaaggccctcaaataagcccctcctgggactccctcaaccccctccattttctccacaaaggccctggtggtttccacattgctacccaatggacacactccaaaatggccagtgggcagggaatcctggagcacttgttccgggatggtgtggtggaagaggggatgagggaaagaaatggggggcctgggtcagatttttattgtggggtgggatgagtaggacaacatatttcagtaataaaatacagaataaaaatcaagtgtttttacgcaaaaaaaaaaaaaaaa

SEQ ID NO: 25 - DNA, 1984 bp, Human
ctgatttacaggaactcacaccagcgatcaatcttccttaatttgtaactgggcagtgtcccgggccagccaatagctaagactgccccccccgcaccccaccctccctgaccctgggggactctctactcagtctgcactggagctgcctggtgaccagaagtttggagtccgctgacgtcgccgcccagatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttggcccctgccacctcctgcctcaggtgtccgctatccaccaaaagggctccctgagggtctgggcaagggacctgcttctattagcccttctccatggccctgccatgctctccaaaccactttttgcagctttctctagttcaagttcaccagactctataaataaaacctgacagaccatgactttcaaaaaaaaaaaaaaaaaa

SEQ ID NO: 26 - DNA, 2620 bp, Human
agatgcgagcactgcggctgggcgctgaggatcagccgcttcctgcctggattccacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaaggttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacagggagagtcagcctccacattcagaggcatcacaagtaatggcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaatatctttccctttgttattgaccatctctgggctttgtatcactaattattttattttattatataataattattttattataataaaatcctgaaaggggaaaataaaaaaaa

SEQ ID NO: 27 - DNA, 2876 bp, Human
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 28 - DNA, 482 bp, Human
gcgggcgccgctcttttgtttcttgctgcagcaacgcgagtgggagcaccaggatctcgggctcggaacgagactgcacggattgttttaagaaaatggcagacaaaccagacatgggggaaatcgccagcttcgataaggccaagctgaagaaaacggagacgcaggagaagaacaccctgccgaccaaagagaccattgagcaggagaagcggagtgaaatttcctaagatcctggaggatttcctacccccgtcctcttcgagaccccagtcgtgatgtggaggaagagccacctgcaagatggacacgagccacaagctgcactgtgaacctgggcactccgcgccgatgccaccggcctgtgggtctctgaagggacccccccccaatcggactgccaaattctccggtttgccccgggatattatagaaaattatttgtatgaataatgaaaataaaacacacctcgtggcatggc

SEQ ID NO: 29 - DNA, 2691 bp, Human
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 30 - DNA, 2775 bp, Human
tgccgcttaataccatcacatgatcctccccgaggccctgtatttaattaaaatagagagggaggcaccacagatgccagaagaacactgttgctcttggtggacgggcccagaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccgtatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggtcagtatttccagaaattgggacgatgttcagtgagagtttctgtgaacacagccaatgtgacacttgggcctcaactcatggaagtgactgtctacagaagacatggacgggcatatgttcccatcgcacaagtgaaagatgtgtacgtggtaacagatcagattcctgtgtttgtgactatgttccagaagaacgatcgaaattcatccgacgaaaccttcctcaaagatctccccattatgtttgatgtcctgattcatgatcctagccacttcctcaattattctaccattaactacaagtggagcttcggggataatactggcctgtttgtttccaccaatcatactgtgaatcacacgtatgtgctcaatggaaccttcagccttaacctcactgtgaaagctgcagcaccaggaccttgtccgccaccgccaccaccacccagaccttcaaaacccaccccttctttagcaactactctaaaatcttatgattcaaacaccccaggacctgctggtgacaaccccctggagctgagtaggattcctgatgaaaactgccagattaacagatatggccactttcaagccaccatcacaattgtagagggaatcttagaggttaacatcatccagatgacagacgtcctgatgccggtgccatggcctgaaagctccctaatagactttgtcgtgacctgccaagggagcattcccacggaggtctgtaccatcatttctgaccccacctgcgagatcacccagaacacagtctgcagccctgtggatgtggatgagatgtgtctgctgactgtgagacgaaccttcaatgggtctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatatttgtcactgtgatctccctcttggtgtacaaaaaacacaaggaatacaacccaatagaaaatagtcctgggaatgtggtcagaagcaaaggcctgagtgtctttctcaaccgtgcaaaagccgtgttcttcccgggaaaccaggaaaaggatccgctactcaaaaaccaagaatttaaaggagtttcttaaatttcgaccttgtttctgaagctcacttttcagtgccattgatgtgagatgtgctggagtggctattaacctttttttcctaaagattattgttaaatagatattgtggtttggggaagttgaattttttataggttaaatgtcattttagagatggggagagggattatactgcaggcagcttcagccatgttgtgaaactgataaaagcaacttagcaaggcttcttttcattattttttatgtttcacttataaagtcttaggtaactagtaggatagaaacactgtgtcccgagagtaaggagagaagctactattgattagagcctaacccaggttaactgcaagaagaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtacagtcgctgcacatatttgaaatcatatattaagactttccaaagatgaggtccctggtttttcatggcaacttgatcagtaaggatttcacctctgtttgtaactaaaaccatctactatatgttagacatgacattctttttctctccttcctgaaaaataaagtgtgggaagagacaagaaaaaaaaaa

SEQ ID NO: 31 - DNA, 2156 bp, Human
ggaggccagtgcgcggccgcggtgctctaccggcgtgtcgctccgccccagggagagccggcgctaccatggaggagtaccatcgccactgcgacgaggttggcttcaatgctgaggaagcccacaatattgtcaaagagtgtgtagatggggttttaggtggtgaagattataatcacaacaacatcaaccagtggactgcaagcatagtggaacaatccttaacacacctggttaagttgggaaaagcctataaatatattgtgacctgtgcagtggtccagaagagcgcatatggctttcacacagccagctcctgtttttgggataccacatctgatggaacctgtaccgtaagatgggagaaccggaccatgaactgtattgtcaacgtttttgccattgctattgttctttaactgactaaaaatgttgggctaaagccattaacttaagaatttgtcagtgtatcctttccaaaaagagtaatagttgtttactagtgtgctagatgaaaagcgtgcaatatgctttaaagctatcaacaaaaactgaatattataagcaagcaatatcatagtaattggcagattagctcatattctatacagcatcgtttaaataggaaaaatttaatgctagcaaaaaataaatttagaatatggcatgacatgaaaatacaatcttatatttacaccagcttttcactaatattttgtacctaaggtgatggggaactccattcagataataaaattctctttcagctagagaagttaacaggaataaatatatgaacaaaaaagctgcaaggataaatgtggagaaaatgatgagaattagctaacatttttaagtttttttaaactttcttcccctcagttgtacttaatatttagtggaaagtaataatttttttattttctatcaactaatagtatagtaacaactatgattaacttgtttactttttctgaggattagtaaatcaattttttttaatttcaaattttggatttacacttgagggtaaattaaatctggtaaactgaatttcctagttaaataaaattagttgcagtatatgatgaacagtgtatgactcaaacagctgccttacaattcactcattccatgtggaacaaacatttatcagatgcctattatgggcatatgtctctgctaagcaccatagttgtcaatgtgctgtgcaaatgctaagttccttttagcaattgttcagttggaagacgtattaatatttggggaaggaaaagaaagtagttgttttacaagggaggaaaaaagtgaatctggttacacatatggaagtaagcaaaatgaaaagcacttattgctttctgacagaattatagatgtaattttaagagttgctcctagcaagttaaaagtgcatataaaatatgcaactcttagttaaaggccttattatcagtcttacctatacaagtagtaaattttgtcattgctttagttacaaccatctgtaaataacttaaaagacttattatgtggggttcaaattgagtggaataaagtatagattaaaagtatacaatccttagcacgttatctcagggcttatgaaatgtaattaaatttattaagaaaatagatgaaaaattagggtacacagctggccaccaaatgcgaagtcaatctgctacttaaccctgaaaacaaaatcagttttgcatattaccactaacactaatacatatagagagcggaaccataactcattgaattttggagaggaataagcttagcgttaatattgacaatattaaggcaatattcttgtaggaatactatgtgcatgtttgatattttgccaaataacaataattaataattgttcaatgtttaagaataatattaacaaaataaaggagtttaatgcagtgatctttgtttttggcacatcaaaaattctcagtcattattcatgtttcttttatgctgctggcttttgtgccctggaagatcataatagtgaccaaaatatacatgcagacttgttttttattattgttgtttaagcataatttaagaaaaaaaatttttacctggtgaacttgctatctgctctgtttctagttaaaatataataaatattatcttcctgtgctgta

SEQ ID NO: 32 - DNA, 1876 bp, Human
ggggaatcctgctctgggatagcacccggccccgcagagcagcgcggcagcccaagggccccggcgccgggggcggcggggaaccccaaacgcaaccgggtctggagggatccccgcgccgagccagccgccgtcaccgcctccgcgccgcccctgcgggcttggcaggcgcccggcgcgcccgcactgcgcccggccgccggctcccgcggtcccaccgtgagctcgccggcccgtcgcccgctcgccatgcaaccgccgccggcctcgcgcgcgtaggcgcccgccgcaggccatgctgcccctgctcgccgcgctcctggccgccgcctgcccgctgccgcccgtccgcggcggggccgcggacgcgcccggcctcctcggggtgccctccaatgcttcagtcaacgcgtcctccgcggacgagcccatcgccccgcggctgctggcctcggcggcccccgggccccccgagcgcccgggcccggaggaggcggcggcggcggcggcgccgtgcaacatcagcgtgcagcggcagatgctgagctcgctgctcgtgcgctggggccgcccgcggggcttccagtgcgacctactgctcttctccaccaacgcgcacggccgcgctttcttcgccgccgccttccaccgcgtcgggccgccgctgctcatcgagcacctggggctggcggcgggcggcgcgcagcaggacctgcgcctctgcgtgggctgcggctgggtgcgcggtcgccgcaccggccgcctccggcccgccgccgcccccagcgccgccgccgccaccgccggggcgcccaccgcgctgccagcctaccccgcggccgagccgcccgggccgctgtggctgcagggcgagccgctgcatttctgctgcctagacttcagcctggaggagctgcagggcgagccgggctggcggctgaaccgtaagcccattgagtccacgctggtggcctgcttcatgaccctggtcatcgtggtgtggagcgtggccgccctcatctggccggtgcccatcatcgccggcttcctgcccaacggcatggaacagcgccggaccaccgccagcaccaccgcagccacccccgccgcagtgcccgcagggaccaccgcagccgccgccgccgccgccgctgccgccgccgccgcggccgtcacttcgggggtggcgaccaagtgacccgctccgctcctccctgtgtccgtcctgtgtccgcgcgcgcgggtgcctttcccgccggagactcggccggtgtgcttcgtgctgtagttatcgttagttcctcttcccgagatggggccgccgagagaccccagcgcctttgaaaagcaaggtttgtgctgcgcttccagttccgaaaagcagatgtttaagcccttggactgagggtgggatcgcagctccgaagacggagaggagggaaatggggccctttcccctctattgcatccccctgcccgactccttccccgcacccacgtgccctagattcatggcagaaaatgaccaaatcctgtgtatttgttttatatatttaataactgttttaaatgaaagttttagtaaaaaaaatacaaaacaaaaagattaaattgctattgctgtagtaagagaagctctttgtatctgaacatagttgtatttgaaatttgtggttttttaatttatttaaaattggggggagggcatgggaaggatttaacaccgatatattgttaccgctgaaaatgaactttatgaaccttttccaagttgatctatccagtgacgtggcctggtgggcgtttcttcttgtacttatgtggttttttggcttttaatacagacattttcctccagaaaaaaaaaaaaaaaaaa

SEQ ID NO: 33 - DNA, 1360 bp, Human
gcccttgccttgagtcagtgcgctgctctccagcccgcttgaacgctccccgcagccaccgccacccattggaatggccaacaggggacctgcatatggcctgagccgggaggtgcagcagaagattgagaaacaatatgatgcagatctggagcagatcctgatccagtggatcaccacccagtgccgaaaggatgtgggccggccccagcctggacgcgagaacttccagaactggctcaaggatggcacggtgctatgtgagctcattaatgcactgtaccccgaggggcaggccccagtaaagaagatccaggcctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcagataaccagctgcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggcctattctgtctctccttcttccccctggcctgttctgtctctgagctctgtgtcctccgttcattccatggctgggagtcactgatgctgcctctgccttctgatgctggactggccttgcttctacaagtatgcttctcccacagctgtggctgcaggaacttaatttatagggaggagcctgtggcagctgctgccccagccacagctgcactgactgtgctcaccacacatctggggcagccttccctggcaggggccctcgtggcttctcattttccattcccttcactgtggctaaggggtggggtgaggggatggagagggagggctgcctaccatggtctggggcttgaggaagatgagtttgttgatttaaataaagaatttgtcatttttg

SEQ ID NO: 34 - DNA, 3398 bp, Human
cgctgtcgccgccagtagcagccttcgccagcagcgccgcggcggaaccgggcgcaggggagcgagcccggccccgccagcccagcccagcccagccctactccctccccacgccagggcagcagccgttgctcagagagaaggtggaggaagaaatccagaccctagcacgcgcgcaccatcatggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgctttgccatccaggacatctccgtggaagagacttcagccaaggaagggctgctcctgtggtgtcagagaaagacagccccttacaaaaatgtcaacatccagaacttccacataagctggaaggatggcctcggcttctgtgctttgatccaccgacaccggcccgagctgattgactacgggaagctgcggaaggatgatccactcacaaatctgaatacggcttttgacgtggcagagaagtacctggacatccccaagatgctggatgccgaagacatcgttggaactgcccgaccggatgagaaagccatcatgacttacgtgtctagcttctaccacgccttctctggagcccagaaggcggagacagcagccaatcgcatctgcaaggtgttggccgtcaaccaggagaacgagcagcttatggaagactacgagaagctggccagtgatctgttggagtggatccgccgcacaatcccgtggctggagaaccgggtgcccgagaacaccatgcatgccatgcaacagaagctggaggacttccgggactaccggcgcctgcacaagccgcccaaggtgcaggagaagtgccagctggagatcaacttcaacacgctgcagaccaagctgcggctcagcaaccggcctgccttcatgccctctgagggcaggatggtctcggacatcaacaatgcctggggctgcctggagcaggtggagaagggctatgaggagtggttgctgaatgagatccggaggctggagcgactggaccacctggcagagaagttccggcagaaggcctccatccacgaggcctggactgacggcaaagaggccatgctgcgacagaaggactatgagaccgccaccctctcggagatcaaggccctgctcaagaagcatgaggccttcgagagtgacctggctgcccaccaggaccgtgtggagcagattgccgccatcgcacaggagctcaatgagctggactattatgactcacccagtgtcaacgcccgttgccaaaagatctgtgaccagtgggacaatctgggggccctaactcagaagcgaagggaagctctggagcggaccgagaaactgctggagaccattgaccagctgtacttggagtatgccaagcgggctgcacccttcaacaactggatggagggggccatggaggacctgcaggacaccttcattgtgcacaccattgaggagatccagggactgaccacagcccatgagcagttcaaggccaccctccctgatgccgacaaggagcgcctggccatcctgggcatccacaatgaggtgtccaagattgtccagacctaccacgtcaatatggcgggcaccaacccctacacaaccatcacgcctcaggagatcaatggcaaatgggaccacgtgcggcagctggtgcctcggagggaccaagctctgacggaggagcatgcccgacagcagcacaatgagaggctacgcaagcagtttggagcccaggccaatgtcatcgggccctggatccagaccaagatggaggagatcgggaggatctccattgagatgcatgggaccctggaggaccagctcagccacctgcggcagtatgagaagagcatcgtcaactacaagccaaagattgatcagctggagggcgaccaccagctcatccaggaggcgctcatcttcgacaacaagcacaccaactacaccatggagcacatccgtgtgggctgggagcagctgctcaccaccatcgccaggaccatcaatgaggtagagaaccagatcctgacccgggatgccaagggcatcagccaggagcagatgaatgagttccgggcctccttcaaccactttgaccgggatcactccggcacactgggtcccgaggagttcaaagcctgcctcatcagcttgggttatgatattggcaacgacccccagggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcgtgtgtgtttgctagctcacttgtccatgaaaatattttatgatattaaagaaaatcttttg

SEQ ID NO: 35 - DNA, 4734 bp, Human
tccggaggcgagccgagcgcggtggtgaggccgcctcagcgaaaaaaatgtccgcctgaagagacccacaagttctattcggggggaccgacagcccgccccgggaggaaggggcggccaggcccgaaagccgcctccccctcccagacccgagagctcgtgcggggcaaagtgaaccgagccgctgggcggtgcaaggggaagcccaagcccgttctcccggccaaagtgaactttaatcggggtggttggatgcggagacggggcggcaggacctgctagaagtggccgaagatgaatccccagcaacaacgcatggccgctatagggaccgacaaggagctgagcgacctactggacttcagtgcgatgttttccccacctgttaatagtgggaaaactagaccaactacactgggaagcagtcaattcagtggatcaggtattgatgaaagaggaggtacaacatcttggggaacaagtggtcaaccaagtccttcctatgattcatctagaggttttacagacagccctcattacagtgatcacttgaatgacagtcgattaggagcccatgaaggcttgtccccaacacctttcatgaactcaaatctgatgggaaaaacatcagagagaggctcattttccctgtacagcagagatactggattaccaggctgtcaatctagtctcctgagacaagatctggggcttgggagcccagcacagctatcttcttcaggaaaacctgggacagcatactattcattctctgctacaagttccaggaggagaccactccatgactctgcagcgcttgatcccttgcaagcaaaaaaagtcagaaaggtgcctcctggtttgccttcttctgtatatgcaccatccccaaattcagatgatttcaaccgtgaatctcctagttatccatctcctaagccaccaaccagtatgttcgctagcactttctttatgcaagatgggacccacaattcttctgacctttggagttcatcaaatgggatgagccagcctggttttggtggaattctggggacctccacttcccacatgtctcaatccagtagttatggcaaccttcattcacatgaccgcttgagttatcctccacactcagtttcaccaacagacataaacacgagtcttccaccaatgtccagctttcatcgcggcagtaccagcagttcaccttacgttgctgcctcacacactcctcccatcaatggatcagacagcattctaggaaccagagggaatgctgctggaagctcacagacaggtgatgcacttggaaaggctttggcatctatttattctcctgaccataccagcagtagttttccgtcaaatccatcaacaccagttggatcaccttcacctctcacaggtaccagtcagtggccaagacctggagggcaagcaccttcatccccaagctatgaaaactcactccactccctgcagtctcgaatggaggatcgtttagacagactggatgatgcaatccatgtgctgcggaaccatgctgtgggaccttccaccagtttgcctgctggtcacagtgatatacatagtttattgggaccatcccataatgcaccaattggaagcctcaattcaaactatggaggatcaagccttgttgcaagcagtcgatcagcttcaatggttggaactcatcgggaagactctgtcagtctcaatggcaatcattcagtcctgtctagtacagtcactacttcaagcacagacctgaaccataaaacacaagaaaattatagaggtggcttgcaaagtcagtctggaactgttgttacaacagaaatcaagactgaaaacaaagaaaaggatgaaaaccttcatgaacctccttcatcagatgacatgaagtcagatgatgaatcctcccaaaaagatatcaaggtttcatctagaggcagaacaagcagtactaatgaagatgaggatttgaaccctgaacagaagatagaaagggagaaggagaggcggatggctaacaatgccagagaacgcttacgcgtgcgggatattaatgaagcattcaaagagcttggccgaatgtgtcagcttcacttgaagagtgaaaaaccccaaacaaaactccttattcttcatcaagccgtggcagtcatccttagtctagaacagcaagtcagagagaggaaccttaaccccaaagcagcctgccttaagagaagggaagaagaaaaagtttctgccgtatcggcagagccgccaaccacactgccaggaacccatcctgggcttagtgaaactaccaaccctatgggtcatatgtaaacatcagccagttccagagttatcagtaggctagatagaaggtgacctctcctcataaggacttggacaactcagattatctgaagacacaaacctgacaggagggagaagaaaaaacaaaacacttgaaccaagaaactcaaatgtaatcctacgatcaaagcaactggtcaacacttccatcagaagtgaagataggaagctcatcagatagaacatcagcccatgagatgtttgcaacaaatcttttgttgcaagcagtgtgtcgcttctgcacaatcagagactgtctcgatctctccactcaccgtggaagttgccttgtgcctaaactgaattgacaaatgcattgtaactacaaattttatttattgttatgaaactgtaaggtctacatataaagggaaaaagttaatgtggaaagctgatctacactcagctgatgccagcatacattaaagcggttcacgtgcagagaacaaagcagtgacaaccattggcccttagcattcccggcatacctattagtgtcttaaaaaggaagggaaaagtcttttgttgccctctcctatcctcttgccatatgaatagcgttttccatgaaataggaaaatattacttggtatagcatttctcttgctctcattttttgatttatttttattttctctttgtgggtgttatatttgatctctaaatctgaacagtttatggtcacagtccagcctcctccgtgcagccctgtgtgctttgcacatttaccttacagtggtaagcagagaccatctgtgaccatagcctagctagcattttaaaaggggaaattttgttctctaggttttcccccaaataaacattgctttatttctaataataaccaagacttttcaagcttctagatctcataggaaagcttgtaatagcaaaattgtaaattacaagggaagaatctactttttagaaatcgctttgttttccaagcagtaagtactacatacagtacttgtaaagtgttagctgtaagtaagcacaaaatacatttaaaatacaaagacgattttttcaggctgtgattatggtgaacataacaaaacccagtagtcaccaaggcaggtagtgtgataaatgaacacaccactctgaggctaattacctaatggaatacaagagcaatggtcacccgtatttccttatcctagcctttatttctctgtcatttggatggctggtcaatggggaagaattgagtgggtgatttaatcaactgcaaaccatctgcccctgtcccaaaatgatgagccagattagcattaaaccagtacttgtcagtccatcttaatactgttcattaaggcactctctgtctctaatccttaggagttgttttaaaagacataatcactttgaacttccatgaaacctgtcttccaccacaacaaccctgggagagaaaaacatgctaaaggaggtatcttggcttaataattccttatagccaatatcaacagtggcaatcagcacacagaggaaaggacccaaatcactatgtagcttaaagatttctgttaatttgaaagaacaaaaacaagacagaacttctggtactctaatcaggatgattcctaacaagtcagtcatttgtgaacttagtggactttttggttactttaatttgcatatattctccagttacatcggactctatctgtggccttgttcttcatttcagtgttaatcagctaaacagaagttgttgcttatgatgtgtgagtgaacatatgccactgcctggcctttttttcttcagagcttgttgtctttttcgctatattagactttgcagtatgcccagaagctttccttcataaaatagaaagaaaaaaacatttggcttatttttcactgtagctagtcttttatacaataatcttgtaagaaaatttcttgaattctaaatattactctttctagatttttgaaatcaaaaagttttcagtaaaaagtttcttactttattttattatattaggtagtaaaaaatgtagggttatttaccataacctgttcattaatatcagaaatttacaatagcattttaagaccatagtaggattctagcataccgtgtagtacctatggagtattgtaagagctaattgttggagatgaattgcttctcatcttgttctccagtttccattgttggtttattgcagatttgtatcctgtgtcaaattcaaggtattattgataaaccttttcaaccagcagcaagaagttcaaatttttttctgtcactgtaacagaaaacacaatatgtatataacatttatgtagcaataaatgtgccatcttttttttaacacagtaaaaaaaaaaaaaaaaaaaaaa

SEQ ID NO: 36 - DNA, 945 bp, Human
ctgggtgtacagcgtcctcgaaaccacgagcaagtgagcagatcctccgaggcaccagggactccagcccatgccatggcggattctgagcgcctctcggctcctggctgctgggccgcctgcaccaacttctcgcgcactcgaaagggaatcctcctgtttgctgagattatattatgcctggtgatcctgatctgcttcagtgcctccacaccaggctactcctccctgtcggtgattgagatgatccttgctgctattttctttgttgtctacatgtgtgacctgcacaccaagataccattcatcaactggccctggagtgatttcttccgaaccctcatagcggcaatcctctacctgatcacctccattgttgtccttgttgagagaggaaaccactccaaaatcgtcgcaggggtactgggcctaatcgctacgtgcctctttggctatgacgcctatgtcaccttccccgttcggcagccaagacatacagcagcccccactgaccccgcagatggcccggtgtaggcgaacttccctcatttctctctgcaatctgcaaataactcctccattgaaataactcctccccaccccaacaacaacattcccagcagaccaactcccaccccctctttgaggtaaaagtgcctttattgggagacttttgtcttccagcctgccaatcaaccctcctgggtgtggccaccatatgtgtgtgcctaggtcctccttctgcacgatccaataggagacaccagttctgactgaaccatgcccccacctaagtcacaaaatgagggaagtggggagttagatttcagagtccaggccctaggttgggacccactccaaataatctcctcggtgtgggtggtggttctatagagggataaatgaataataaacattgttaaaatataaaaaaaaaaaaaaaaa

SEQ ID NO: 37 - DNA, 1920 bp, Human
agctctgcagtcctcctatgtggtactgatcaggtggttgcagagcttcagctcacagcaacacaatgcagctgagcaggcaagcacagcccacagccagaaacagttccgactctacagaacaagacgacctttaagtttcccagagaaaatgagatgctgatgttgaagacgacaccacggctttgatggaatatcagatattgaaaatgtctctctgcctgttcatccttctgtttctcacacctggtattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctgagggtctgcattttctgaaactaatgaaagcactcctccctgatgtacagttgggaaaatatgtccatatctaaccagtgattcgagctatatttaagtattcaagaaagccagtcttaacatttctaactctgatgtaaatgaagtaacttgtcttaaataaaagaaatgcacaatgtcttggtacttgctgctattttactgtcttaattaagtaaactaatgagtttcttttataaaaaaaatgaaatgttttaaggcttcaatttattgcacaaaatataaagcatctaaactttaatatgtattttatgtatgtttacactgtcaaacatctggaaaataaaaggtctatgctcaaaaaaaaaaaaaaaaaaaaaaaaaaaaaaaaaaaaaaaaaaaaaaaaaaa

SEQ ID NO: 38 - DNA, 1069 bp, Human
aagtaattcctagacccgtaggtggccgcagagccggttacctctggttctgcgccagcgtgccccacccgcaggacggccgggttctttgatttgtacactttctaaaaccaaacccgagaggaagggcaggctcagggtggggatgccctgaaatattcgagagcaggaccgtttctactgaagagaagtttacaagaacgctctgtctggggcgggcgaggcctctgcgaggcgggtccgggagcgagggcagggcgtgggccgcgcgcccggggtcgggggagtcgggggcaggaagagggggaggagacagggctgggggagcgccctgccgagcgcccgccaggctcctcccgctcccgcgccgcctccctctacccacccgccgcacgtactaaggaaggcgcacagcccgccgcgctcgcctctccgccccgcgtccagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtcccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttccaaatttgaaaaaaaaaaaaaaa

SEQ ID NO: 39 - DNA, 21 bp, Artificial Sequence
  Synthetic / Artificial Primer
tggcagagaagtacctggaca

SEQ ID NO: 40 - DNA, 23 bp, Artificial Sequence
  Synthetic / Artificial Primer
gacaccaacaagattgaggaatt

SEQ ID NO: 41 - DNA, 19 bp, Artificial Sequence
  Synthetic / Artificial Primer
gagcgagggacaagactcc

SEQ ID NO: 42 - DNA, 23 bp, Artificial Sequence
  Synthetic / Artificial Primer
caagaaaattgaaagatgggaaa

SEQ ID NO: 43 - DNA, 21 bp, Artificial Sequence
  Synthetic / Artificial Primer
gccactggagtctttaccaca

SEQ ID NO: 44 - DNA, 20 bp, Artificial Sequence
  Synthetic / Artificial Primer
gggaagcttgtcatcaatgg

SEQ ID NO: 45 - DNA, 20 bp, Artificial Sequence
  Synthetic / Artificial Primer
tgcaagattgccacttgatg

SEQ ID NO: 46 - DNA, 19 bp, Artificial Sequence
  Synthetic / Artificial Primer
gtccttggggaacatggag

SEQ ID NO: 47 - DNA, 18 bp, Artificial Sequence
  Synthetic / Artificial Primer
gagagagcagcccgagag

SEQ ID NO: 48 - DNA, 21 bp, Artificial Sequence
  Synthetic / Artificial Primer
acgacaccacggctttgatgg

SEQ ID NO: 49 - DNA, 18 bp, Artificial Sequence
  Synthetic / Artificial Primer
gggtcctggcagaaggag

SEQ ID NO: 50 - DNA, 20 bp, Artificial Sequence
  Synthetic / Artificial Primer
gacctgcacaccaagatacc

SEQ ID NO: 51 - DNA, 18 bp, Artificial Sequence
  Synthetic / Artificial Primer
agttccctggatttttgg

SEQ ID NO: 52 - DNA, 20 bp, Artificial Sequence
  Synthetic / Artificial Primer
tcacaggggccaggaaccta

SEQ ID NO: 53 - DNA, 21 bp, Artificial Sequence
  Synthetic / Artificial Primer
aaggcacctctgagaacttca

SEQ ID NO: 54 - DNA, 18 bp, Artificial Sequence
  Synthetic / Artificial Primer
gaccctgctgaccctcct

SEQ ID NO: 55 - DNA, 20 bp, Artificial Sequence
  Synthetic / Artificial Primer
ggccaaggctctactgtctg

SEQ ID NO: 56 - DNA, 15 bp, Artificial Sequence
  Synthetic / Artificial Primer
ccagcccgcttgaac

SEQ ID NO: 57 - DNA, 24 bp, Artificial Sequence
  Synthetic / Artificial Primer
ccctgtacagcagagatactggat

SEQ ID NO: 58 - DNA, 20 bp, Artificial Sequence
  Synthetic / Artificial Primer
cagaagagcgcatatggctt

SEQ ID NO: 59 - DNA, 20 bp, Artificial Sequence
  Synthetic / Artificial Primer
cttcaagcatcgtgttgagc

SEQ ID NO: 60 - DNA, 18 bp, Artificial Sequence
  Synthetic / Artificial Primer
ctgccgaccaaagagacc

SEQ ID NO: 61 - DNA, 22 bp, Artificial Sequence
  Synthetic / Artificial Primer
gacgatgcacactttaattagc

SEQ ID NO: 62 - DNA, 20 bp, Artificial Sequence
  Synthetic / Artificial Primer
ggcagttccaacgatgtctt

SEQ ID NO: 63 - DNA, 18 bp, Artificial Sequence
  Synthetic / Artificial Primer
gccagcttggggtacctg

SEQ ID NO: 64 - DNA, 19 bp, Artificial Sequence
  Synthetic / Artificial Primer
gacatggctgcagtggaag

SEQ ID NO: 65 - DNA, 22 bp, Artificial Sequence
  Synthetic / Artificial Primer
ccgagtacaggtgacattgttc

SEQ ID NO: 66 - DNA, 20 bp, Artificial Sequence
  Synthetic / Artificial Primer
cctcggtgttgtaaggtgga

SEQ ID NO: 67 - DNA, 20 bp, Artificial Sequence
  Synthetic / Artificial Primer
ttgattttggagggatctcg

SEQ ID NO: 68 - DNA, 22 bp, Artificial Sequence
  Synthetic / Artificial Primer
ccctcatgtaagcagaaggtct

SEQ ID NO: 69 - DNA, 21 bp, Artificial Sequence
  Synthetic / Artificial Primer
gacaccagcaacattcattcc

SEQ ID NO: 70 - DNA, 20 bp, Artificial Sequence
  Synthetic / Artificial Primer
gactgccagatttcatcctc

SEQ ID NO: 71 - DNA, 21 bp, Artificial Sequence
  Synthetic / Artificial Primer
ccaggtgtgagaaacagaagg

SEQ ID NO: 72 - DNA, 20 bp, Artificial Sequence
  Synthetic / Artificial Primer
cgccttccaaacctgtagtc

SEQ ID NO: 73 - DNA, 19 bp, Artificial Sequence
  Synthetic / Artificial Primer
cgctatgagggttcggaag

SEQ ID NO: 74 - DNA, 17 bp, Artificial Sequence
  Synthetic / Artificial Primer
tggtccaggtccttcat

SEQ ID NO: 75 - DNA, 22 bp, Artificial Sequence
  Synthetic / Artificial Primer
tgccctcctcaaatacatcaag

SEQ ID NO: 76 - DNA, 18 bp, Artificial Sequence
  Synthetic / Artificial Primer
cccaggactaggcaggtg

SEQ ID NO: 77 - DNA, 20 bp, Artificial Sequence
  Synthetic / Artificial Primer
ggagctggtagcatttggat

SEQ ID NO: 78 - DNA, 19 bp, Artificial Sequence
  Synthetic / Artificial Primer
ccatgtctggggaaagctc

SEQ ID NO: 79 - DNA, 17 bp, Artificial Sequence
  Synthetic / Artificial Primer
caggccatatgcaggtc

SEQ ID NO: 80 - DNA, 20 bp, Artificial Sequence
  Synthetic / Artificial Primer
aagccccagatcttgtctca

SEQ ID NO: 81 - DNA, 20 bp, Artificial Sequence
  Synthetic / Artificial Primer
cttacggtacaggttccatc

SEQ ID NO: 82 - DNA, 20 bp, Artificial Sequence
  Synthetic / Artificial Primer
gacacctttgagacccttcg

SEQ ID NO: 83 - DNA, 19 bp, Artificial Sequence
  Synthetic / Artificial Primer
gggtaggaaatcctccagg

SEQ ID NO: 84 - DNA, 20 bp, Artificial Sequence
  Synthetic / Artificial Primer
gaagttggtttttcctctcc

SEQ ID NO: 85 - DNA, 19 bp, Artificial Sequence
  Synthetic / Artificial Primer
agtgtgtgcccactgagga

SEQ ID NO: 86 - DNA, 20 bp, Artificial Sequence
  Synthetic / Artificial Primer
ggtgaggtttgatccgcata

(19) United States
(12) Patent Application Publication
(10) Pub. No.: US 2010/0167939 A1
(43) Pub. Date: Jul. 1, 2010

(54) MULTIGENE ASSAY TO PREDICT OUTCOME IN AN INDIVIDUAL WITH GLIOBLASTOMA

(76) Inventors: Kenneth Aldape, Houston, TX (US); Howard Colman, Houston, TX (US); Li Zhang, Bellaire, TX (US)

Correspondence Address: FULBRIGHT & JAWORSKI, LLP, 1301 MCKINNEY, SUITE 5100, HOUSTON, TX, 77010-3095, US

(21) Appl. No.: 12/529,628

(22) PCT Filed: Feb. 29, 2008
(86) PCT No.: PCT/US08/55472

§ 371 Date: Jan. 14, 2010

Related U.S. Application Data
(60) Provisional application No. 60/892,825, filed on Mar. 2, 2007.

(51) Int. Cl. C40B 30/00; C12Q 1/68; C12Q 1/02; C40B 40/06

(52) U.S. Cl. 506/7; 435/6; 435/29; 506/16

(57) ABSTRACT

The present invention concerns prognosis for glioblastoma and/or assessment of the response of an individual to therapy for glioblastoma treatment. In particular, expression analysis of two or more specific genes provided in the invention is determined to predict outcome for the individual and/or to predict if the individual will respond to therapy, such as chemoradiation, for example. In specific embodiments, a multigene set from a sample from the individual is compared to a reference set of housekeeping genes.

The present invention claims priority to U.S. Provisional Patent Application Ser. No. 60/892,825, filed Mar. 2, 2007, which is incorporated by reference herein in its entirety.

### FIELD OF INVENTION

The present invention concerns at least the fields of molecular biology, cell biology, and medicine, in particular cancer therapy and/or prognosis. In specific embodiments, the present invention concerns gene expression analysis to identify prognosis and/or therapy response for individuals with glioblastoma.

### BACKGROUND OF THE INVENTION

Glioblastoma (GBM) is the most common primary brain tumor in adults and is highly lethal (Kleihues et al., 2000) The majority of GBM patients are treated with surgery, radiation and some alkylator-based chemotherapy. Despite increasing evidence that distinct molecular subtypes of GBM exist (Burton et al., 2002; Hegi et al., 2005; Freije et al., 2004; Nigro et al., 2005; Haas-Kogan et al., 2005; Mellinghoff et al., 2005) patients are generally treated in a uniform fashion. However, correlative studies to a recent phase III clinical trial comparing TMZ plus radiation versus radiation alone (Stufpp et al., 2005) showed that methylation of the MGMT promoter was associated with prolonged survival compared to non-methylated cases (Hegi et al., 2005). Patients whose tumors displayed MGMT promoter methylation exhibited a 34.4% 2-year survival rate, while those without MGMT methylation had a 2-year survival rate of 8.2%. This marker was associated with better 2-year survival in both the TMZ-treated arm (46.0% vs. 13.8% for methylated versus unmethylated, respectively) as well as the radiation-only arm (22.7% vs. <2%). While promising as a marker, over half (54%) of the patients in the favorable treatment arm (TMZ) whose tumors were MGMT-methylated did not survive 2 years. These data are promising, but the identification of additional predictors to more precisely distinguish those individuals who will and will not experience a durable response to standard therapy is needed.

Expression microarray analysis provides a rich source of potential biomarkers for clinical use (Paik et al., 2004; Fan et al., 2006; Potti et al., 2006). However, the large number of genes investigated relative to the comparatively small number of samples results in a high false discovery rate in individual datasets (Ransohoff et al., 2004; Simon, 2005) and generalizations from single microarray datasets must therefore be made with caution (Shi et al., 2006). Several studies examining gene expression profiles associated with clinical outcome in GBM have been published (Nigro et al., 2005; Liang et al., 2005; Nutt et al., 2003; Phillips et al., 2006; Rich et al., 2005) with notable differences in the top reported survival-associated genes. Furthermore, no consensus gene expression profile reproducibly associated with patient outcome across independent datasets has been identified for GBM. In this invention, a meta-analysis of gene expression array data was conducted from multiple institutions to identify a robust multigene predictor of outcome in GBM. This multigene predictor is further characterized in an independent set of GBM tumors.

### SUMMARY OF THE INVENTION

The present invention generally concerns prognosis and/or therapy response outcome for one or more individuals with glioblastoma. The present invention provides a set of genes, the expression of which has at least prognostic value, specifically with respect to survival, for example disease-free survival and/or response to therapy. Currently, there is no test to predict outcome in glioblastoma, such as wherein one can stratify individuals with glioblastoma into good versus poor responders. As a consequence, some individuals may unnecessarily receive treatment for which their tumor is resistant or will become resistant. Alternatively some individuals may be undertreated, in that additional agents added to standard therapy may improve outcome for these patients who would be refractory to standard treatment alone. Since treatment with each additional agent involves additional toxicity, it would be important not to overtreat such patients who might respond to current standard therapy without such additional agents in the treatment regimen. Therefore it would be desirable to prospectively distinguish responders from non-responders to standard therapy prior to the initiation of therapy in order to optimize therapy for individual patients. In certain embodiments of the invention, there is provided a multigene classifier predictive of outcome in glioblastoma, including newly diagnosed glioblastoma. In some embodiments, there is a multigene predictor for individualization of treatment for one or more individuals with glioblastoma, including those newly diagnosed with glioblastoma.

In specific embodiments, the invention provides a clinical test that is useful to predict outcome in glioblastoma. The expression of specific cancer genes is measured in the tumor tissue, for example. Individuals are stratified into those who are likely to respond well to therapy vs. those who will not. A health care provider uses the results of the test to help determine the best therapy for the individual in need of therapy. Individuals are stratified into those who are likely to have a poor prognosis vs. those who will have a good prognosis with standard therapy. A health care provider uses the results of the test to help determine the course of action, for example the best therapy, for the individual in need of therapy.

In specific aspects, a test is provided whereby a tumor is profiled for a multigene set and, from the results, an estimate of the likelihood of response to standard glioblastoma (GBM) therapy therapy is determined.

In another embodiment, the invention concerns a method of predicting the prognosis and/or likelihood of response to standard radiation-chemotherapy, following treatment, in an individual with glioblastoma, comprising determining the expression level of the multigene set in a cancer tissue obtained from the individual, normalized against a control gene or genes. A total value is computed for each individual from the expression levels of the individual genes in this multigene set. To estimate likelihood of response, the value of the multigene profile in a test sample will be compared to a reference set in the following exemplary way: a set of glioblastoma samples from patients, for example 100 glioblastoma samples from patients, with known clinical outcome are tested by the multigene test. Since the 2-year survival rate for patients with glioblastoma treated with current standard therapy is approximately 25%, this value will be used as the cutoff to determine risk. The samples in the reference set are analyzed to confirm that 1) all patients were treated with current standard therapy; and 2) approximately 25% of tumors come from patients who survived more than 2 years. Therefore a test value is compared to the values found in a reference glioblastoma tissue set, wherein a collective expression level in about the upper 75th percentile indicates an increased risk of poor prognosis and/or poor response to radiation-chemotherapy and a collective expression level in about the lower 25th percentile indicates an increased chance of good prognosis and/or good response to radiation-chemotherapy.

In particular, the use of expression microarray data to distinguish molecular subtypes of tumors associated with distinct clinical outcomes is useful for both identification of novel therapeutic targets and individualization of treatment based on molecular profile. However, a significant limitation in the use of microarray data from an individual study to prospectively identify robust predictors of outcome is that the high number of genes investigated combined with a relatively low number of samples results in a high false discovery rate. This leads to a correspondingly low likelihood that the top survival genes observed in one study will predict outcome in an independent set of samples. To overcome this problem, the inventors conducted a meta-analysis by combining Affymetrix expression array data from 4 different institutions comprising 110 cases of newly diagnosed glioblastoma (GBM). Algorithms were developed for merging data from different Affymetrix chips (U133A and U95A), data normalization, removal of institutional bias, and identification of samples having significant contamination of normal brain tissue. The top 200 survival genes were identified from each of the 4 data sets individually using the fold-change between the typical GBM survivor group (less than 2 years) versus the long-term survivor group (2 years or greater). Using an iterative “leave-one-institution out” approach, it was found that a gene expression signature consisting of the top 200 genes with the highest fold-change between survival groups from any 3 institutions (training set) could predict survival in the remaining fourth data set (test set). It was next determined the most robust consensus set by identifying the top survival genes common to all 4 datasets. This analysis identified 38 genes that were ranked in the top 200 in data from all 4 institutions, a result found to be highly unlikely due to chance. A composite survival index derived from these 38 genes predicted survival in all 4 datasets. These findings indicate that gene expression profiles derived from one GBM data set can predict survival in an independent dataset and that a consensus multigene survival classifier for GBM can be identified. An exemplary clinical test for prognosis and treatment response prediction in GBM is provided.

Thus, in some embodiments of the invention, there are methods to screen one or more individuals for the prognosis for glioblastoma in the one or more individuals. The invention may provide information concerning the survival rate of an individual, the predicted life span of the individual, and/or the predicted likelihood of survival for the individual (all wherein the survival may be long-term survival), and so forth, in certain aspects. In specific embodiments, a survival of greater than about two years is referred to as a long-term survival.

In other cases, the invention may also determine if an individual will respond to one or more therapies for glioblastoma. The therapy may be of any kind, but in specific embodiments it comprises chemotherapy, such as one or more alkylating agents, and/or radiation. In specific embodiments, the chemotherapy comprises temozolomide, carmustine, cyclophosphamide, procarbazine, lomustine, and vincristine, carboplatin, and/or irinotecan.

In one embodiment of the invention, expression of nucleic acid markers is used to select clinical treatment paradigms for brain cancer. Treatment options, as described herein, may include but are not limited to chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration. Another treatment for glioblastoma is surgery, which can be utilized either alone or in combination with any of the aforementioned treatment methods. One of ordinary skill in the medical arts may determine an appropriate treatment paradigm based on evaluation of differential expression of sets of two or more of the nucleic acid targets as exemplified by SEQ ID NOS. 1-38. Cancers that express markers that are indicative of a more aggressive cancer or poor prognosis may be treated with more aggressive therapies, in specific embodiments. Cancers that express markers that are indicative of being a poor responder to one or more therapies may be treated with one or more alternative therapies, in specific embodiments.

In some embodiments of the invention, there is a method of predicting the likelihood of long-term survival of individual with glioblastoma, comprising determining the expression level of two or more of the RNA transcripts of the genes in Table 4 or their expression products (which may be referred to as a protein translation product, or just protein, in certain embodiments) in at least one cell obtained from the individual, normalized against the expression level of a reference set of RNA transcripts or their expression products from the cell or the expression levels of all RNA transcripts or their expression products in the cell, wherein the expression levels from the two or more genes provides information about long-term survival and/or response to therapy, such as radiation and/or chemotherapy.

In other embodiments, there is a method of predicting the likelihood of long-term survival of an individual diagnosed with glioblastoma, comprising the steps of (a) determining the expression levels of the RNA transcripts of two or more of the genes in Table 4, or their expression products, in a cell obtained from the individual, normalized against the expression levels of all RNA transcripts or their expression products in said cell, or of a reference set of RNA transcripts or their products from the cell; (b) subjecting the data obtained in step (a) to statistical analysis; and; (c) determining whether the likelihood of said long-term survival has increased or decreased.

In additional embodiments, there is a method of preparing a personalized genomics profile for an individual with glioblastoma, comprising the steps of (a) subjecting RNA extracted from a cancer cell of the individual to gene expression analysis; (b) determining the expression level in the tissue of the RNA transcripts of two or more genes in Table 4, wherein the expression level is normalized against a control gene or genes and may be compared to the amount found in a glioblastoma reference tissue set; and (c) generating a report of the data obtained by the gene expression analysis, wherein the report comprises a prediction of the likelihood of long term survival of the individual or a response to therapy.

In various embodiments, the expression level of at least about 2, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 22, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37 prognostic RNA transcripts or their expression products from the genes listed in Table 4 is determined.

In a still further embodiment, the expression level of one or more prognostic RNA transcripts, or their expression products, of one or more genes selected from the group consisting of the genes listed in Table 4 is determined, wherein increased expression of one or more of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10 indicates poor prognosis and therefore a decreased likelihood of long-term survival without cancer recurrence and/or wherein decreased expression of one or more of KIAA0509, RTN1, GRIA1, GABBR1, OLIG2, TCF12, and OMG indicates good prognosis and therefore an increased likelihood of long-term survival without cancer recurrence.

In a different embodiment, the invention concerns a combined RT-PCR test involving 1 or more of the following genes: TIMP1, CHI3L1, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFBI, GPNMB, TCTE1L, RIS1, TAGLN2, ACTN1, PLP2, PBEF, LTF1, CHI3L2, SEC61G, DKFZp564K0822, EGFR, and S100A10, whose elevated expression levels indicate poor response to therapy; as well as one or more of the following genes: KIAA0509, RTN1, GRIA2, GABBR1, OLIG2, TCF12, OMG, C10orf56, ID1, PDGFRA, and C1QL1, whose elevated expression levels indicate good response to therapy.

In specific embodiments of the invention, prognostic information for the prediction of patient outcome is obtained from expression levels of one or more of the following: PDPN, AQP1, YKL40, GPNMB, EMP3, S100, IGFBP2, LGALS3, SERPE3, TNC, NNMT, VEGFA, TCTEIL, MAOB, TAGLN2, RTN1, KIAA0510, OLIG2, GABA, EGFR, CHI3L2, C1QL1, PDGFRA, ID1, and LTF.

In another embodiment, the invention concerns a collection of nucleic acids, for example an array, comprising polynucleotides hybridizing under stringent conditions to two or more of polynucleotides of the genes or their complements listed in Table 4. In a further embodiment, the array comprises polynucleotides hybridizing to at least 3, or at least 5, or at least 10, or at least 15, or at least 20, or at least 25 of the listed genes. In a still further embodiment, the arrays comprise polynucleotides hybridizing to all of the listed genes. In yet another embodiment, the arrays comprise more than one polynucleotide hybridizing to the same gene. In an additional embodiment, the arrays comprise intron-based sequences. In another embodiment, the polynucleotides are cDNAs, which can, for example, be about 500 to about 5000 bases long. In yet another embodiment, the polynucleotides are oligonucleotides, which can, for example, be about 10 to about 80 bases long. The arrays can, for example, be immobilized on glass, plastic, or another substrate material, and can comprise many oligonucleotides.

In a further aspect, the invention concerns a method for measuring levels of mRNA products of genes listed in Table 4 by real time polymerase chain reaction (RT-PCR), by using a primer-probe set listed in at least Table 2.

All types of cancer are included, such as, for example, brain cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, and melanoma. The foregoing methods are particularly suitable for prognosis/classification of brain cancer, such as glioblastoma.

The individual of the invention may be a mammal, for example a human, dog, cat, horse, cow, or sheep.

In some embodiments of the invention, there is a method of screening an individual for glioblastoma prognosis and/or response to glioblastoma therapy, comprising the step of analyzing the expression levels of two or more genes in Table 4 from a sample from the individual. In a certain aspect, the method is screening an individual for glioblastoma prognosis, and in an additional or alternative aspect the method is screening an individual for response to glioblastoma therapy. In specific embodiments, the expression levels of RNA or protein are analyzed. In specific embodiments, the method is further defined as determining the expression level of the RNA transcripts of two or more of the genes listed in Table 4, or their expression products, from a cell obtained from a sample from said individual, wherein said level is normalized against the expression level of one or more genes in a reference set of RNA transcripts, or their expression products.

In certain cases, a reference set, which may be referred to as a reference gene set, comprises one or more housekeeping genes. In a specific embodiment, the glioblastoma therapy comprises radiation, chemotherapy, or a combination thereof. The chemotherapy may be further defined as comprising one or more alkylating agents. In some cases, the chemotherapy comprises temozolomide, carmustine, cyclophosphamide, procarbazine, lomustine, and vincristine, carboplatin, irinotecan, erlotinib, sorafenib, RAD001, or a combination thereof. In specific embodiments, the analyzing comprises polymerase chain reaction, microarray analysis, or immunoassay.

In other embodiments, there is an isolated collection of nucleic acids comprising no more than the following: a) the genes listed in Table 4; and b) no more than about five housekeeping genes. In certain embodiments, the collection is further defined as comprising in a) about 95% of the genes listed in Table 4, about 90% of the genes listed in Table 4, about 80% of the genes listed in Table 4, about 75% of the genes listed in Table 4, about 70% of the genes listed in Table 4, about 60% of the genes listed in Table 4, about 55% of the genes listed in Table 4, about 50% of the genes listed in Table 4, about 45% of the genes listed in Table 4, about 40% of the genes listed in Table 4, about 35% of the genes listed in Table 4, about 30% of the genes listed in Table 4, about 25% of the genes listed in Table 4, about 20% of the genes listed in Table 4, about 15% of the genes listed in Table 4, about 10% of the genes listed in Table 4, or about 5% of the genes listed in Table 4. In particular cases, the collection is housed on a substrate. In other particular cases, the housekeeping genes are selected from the group consisting of glyceraldehyde-3-phosphate-dehydrogenase (GAPDH), β-glucuronidase, actin, ubiquitin, albumin, cytochrome, and tubulin.

In some embodiments of the invention, there is a method of screening an individual for glioblastoma prognosis and/or response to glioblastoma therapy, comprising assessing the expression levels of the RNA transcripts of the genes listed in Table 4, or their expression products, in a glioblastoma cell sample from the individual, as normalized in relation to the expression levels of one or more reference RNA transcripts, or their expression products, and determining a prognosis or therapeutic response by means of said comparison. The assessing may comprise polymerase chain reaction, microarray analysis, or immunoassay, for example.

In specific embodiments, there is increased expression, as compared to the reference RNA transcripts, of one or more of KIAA0509, RTN1, GRIA1, GABBR1, OLIG2, TCF12, C10orf56, ID1, PDGFRA, C1QL1 and OMG that indicates a favorable prognosis and/or favorable response to therapy, and/or increased expression, as compared to the reference RNA transcripts, of one or more of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10, PBEF, LTF1, CHI3L2, SEC61G, DKFZp564K0822, and EGFR that indicates an unfavorable prognosis and/or unfavorable response to therapy.

In an additional embodiment of the invention, there is a method of the invention may be further defined as: (a) determining the expression levels of RNA transcripts from two or more genes listed in Table 4; (b) normalizing the expression levels of the RNA transcripts from two or more genes to expression levels of one or more reference RNA transcripts; (c) subtracting the sum of the normalized expression values for the RNA transcripts from genes associated with favorable prognosis and/or therapy response from the sum of the normalized expression values for the RNA transcripts from genes associated with unfavorable prognosis and/or therapy response, wherein said subtracting results in a tumor value; (d) comparing the tumor value with reference glioblastoma tumor values, wherein a tumor value that is in the upper 75th percentile relative to the reference glioblastoma tumor values indicates an unfavorable prognosis and/or therapy response and wherein a tumor value that is in the lower 25th percentile relative to the reference glioblastoma tumor values indicates a favorable prognosis and/or therapy response, wherein the genes associated with favorable prognosis and/or therapy response are selected from the group consisting of KIAA0509, RTN1, GRIA1, GABBR1, OLIG2, TCF12, C10orf56, ID1, PDGFRA, C1QL1 and OMG, and wherein the genes associated with unfavorable prognosis and/or therapy response are selected from the group consisting of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10, PBEF, LTF1, CHI3L2, SEC61G, DKFZp564K0822, and EGFR.

In specific embodiments, one or more genes listed in Table 4 are further defined as being selected from the group consisting of PDPN, AQP1, YKL40, GPNMB, EMP3, S100, IGFBP2, LGALS3, SERPE3, TNC, NNMT, VEGFA, TCTEIL, MAOB, TAGLN2, RTN1, KIAA0510, OLIG2, GABA, EGFR, CHI3L2, C1QL1, PDGFRA, ID1, and LTF.

In specific aspects of the invention, genes associated with favorable prognosis and/or favorable therapy response are involved in mesenchymal differentiation, extracellular matrix, or angiogenesis, whereas genes associated with unfavorable prognosis and/or unfavorable therapy response are involved in neural development.

In one specific case, the method of the invention is for screening an individual for glioblastoma prognosis. In another specific case, the method of the invention is screening an individual for response to glioblastoma therapy, such as therapy that comprises radiation, chemotherapy, or a combination thereof. The chemotherapy may be further defined as comprising one or more alkylating agents, and the chemotherapy may be defined as comprising temozolomide, carmustine, cyclophosphamide, procarbazine, lomustine, and vincristine, carboplatin, irinotecan, erlotinib, sorafenib, RAD001, or a combination thereof.

Reference RNA transcripts of the invention may be of any suitable kind, for example RNa transcripts having relatively consistent expression levels, but in specific embodiments the reference RNA transcripts are from one or more housekeeping genes, such as those selected from the group consisting of glyceraldehyde-3-phosphate-dehydrogenase (GAPDH), β-glucuronidase, actin, ubiquitin, albumin, cytochrome, and tubulin.

In an additional embodiment of the present invention, there is a kit comprising an isolated collection of nucleic acids that hybridize under stringent conditions to the RNA transcripts from at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, or 38 of the genes listed in Table 4. In particular aspects of the kit, the nucleic acids hybridize under stringent conditions to RNA transcripts from at least 2, 3, 4, 5, 6, 7, 8, 9, 10, 11, 12, 13, 14, 15, 16, 17, 18, 19, 20, 21, 22, 23, or 24, or from all of the genes selected from the group consisting of PDPN, AQP1, YKL40, GPNMB, EMP3, S100, IGFBP2, LGALS3, SERPE3, TNC, NNMT, VEGFA, TCTEIL, MAOB, TAGLN2, RTN1, KIAA0510, OLIG2, GABA, EGFR, CHI3L2, C1QL1, PDGFRA, ID1, and LTF.

In specific cases, the kit further comprises nucleic acids that hybridize under stringent conditions to RNA transcripts from 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer housekeeping genes. In additional specific cases, the housekeeping genes are selected from the group consisting of glyceraldehyde-3-phosphate-dehydrogenase (GAPDH), β-glucuronidase, actin, ubiquitin, albumin, cytochrome, and tubulin.

In particular embodiments of the kit, the isolated collection of nucleic acids are housed on a substrate, such as a microarray chip, membrane, or column, for example.

In another embodiment of the invention, there is a collection of oligonucleotides, wherein each of the oligonucleotides hybridizes under stringent conditions to an RNA transcript from a gene listed in Table 4. The oligonucleotides may be further defined as primers for polymerase chain reaction, in certain embodiments.

The collection may comprise 1 or more, 2 or more, 3 or more, 4 or more, 5 or more, or 6 or more primers for an RNA transcript from each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or all 38 genes listed in Table 4.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The attached drawings form part of the present specification and are included to further demonstrate certain aspects of the present invention. The invention may be better understood by reference to one or more of these drawings in combination with the detailed description of specific embodiments presented herein.

FIG. 1 illustrates the exemplary scheme used to identify robust survival genes in independent microarray datasets derived from MD Anderson (MDA), Massachusetts General Hospital (MGH), University of California-Los Angeles (UCLA) and University of California-San Francisco (UCSF).

FIG. 2 shows an exemplary test of robustness of gene expression sets among institutions using a “leave-one-institution-out” cross validation method. Data were combined from 3 institutions into a single dataset, and the list of the top 200 survival genes identified among those 3 institutions (the training set). This list of genes was then used for K-means clustering of the dataset from 4th institution (the test set). The survival times are plotted for the 2 groups that resulted from the clustering analysis. This procedure was repeated for all (n=4) possible combinations of the datasets and the resulting Kaplan-Meier curves for the test set in each case shown in A-D. All log rank tests were significant (p<0.05) except for 4C, where p=0.09.

FIGS. 3A-3D demonstrate identification of robust outcome-associated genes from microarray data. In FIG. 3A, overlap of survival genes among 4 microarray datasets is shown. The top 200 genes were identified for each dataset individually and the overlap of the 4 lists is shown in a Venn diagram. FIG. 3B shows estimation of false discovery rate. The survival data was scrambled among the samples and a list of 200 genes was generated from each dataset using the scrambled survival data. The typical overlap of genes resulting from repeating this exercise 5 times is shown. FIG. 3C shows survival according to metagene score. The 38 survival-associated genes common to all 4 datasets were used to calculate a metagene score for each sample. The metagene score was calculating by subtracting the sum of the values of the good-prognosis genes from the sum of the values of the poor-prognosis genes. The samples were ranked by metagene score and divided into quarters. Survival according to metagene score is shown for the bottom quarter (red) vs. the remaining samples (blue). FIG. 3D shows radiation response according to metagene score. A subset (n=23) of samples for which pre- and post-radiation therapy images were available was assessed for response to radiation as a function of metagene score. Patients were scored as progressors (−1) versus stable (0) versus responders (+1). The average radiation score was calculated for patients whose tumors were in the bottom quarter of metagene scores compared to the remainder.

FIGS. 4A-4D show validation and optimization of multigene predictor in an independent sample set. A set of 69 formalin-fixed, paraffin embedded glioblastoma samples were subject to qRT-PCR for the 38 gene set identified in FIG. 3. FIG. 4A shows that a metagene score was calculated as in FIG. 3 and the samples ranked by metagene score. Survival is shown for the bottom quarter of metagene scores (red) versus the remaining samples (blue). In FIG. 4B, a classifier was determined from a subset (n=6) of the 38 genes assays using a logistic regression model. Classifier scores were ranked and survival is shown for the top quarter vs. the remaining samples. FIGS. 4C and 4D provide metagene scores and response to radiation. Pre- and post-radiation studies were available on 53/69 patients. Radiation response scores were calculated as in FIG. 3, and are shown as function of metagene scores for: 4C. entire 38-gene set; 4D. 6-gene set.

FIG. 5 shows consistency of gene rankings across institutions: Individual genes were ranked by fold change or SAM 2-class (TS vs. LTS) within each institution. Average rank and standard deviation of gene ranks across the 4 microarray data sets were calculated. The standard deviation as a function of average gene rank are plotted for the top 1000 genes (top row) or top 200 genes (bottom row) for Fold Change and SAM. The lower standard deviation observed across all rankings using fold change indicated that this method gave more consistent rankings of individual genes across institutions and fold change was thus chosen as the method used to identify the most robust survival genes common to the independent data sets.

FIG. 6 shows survival by classifier score quarters. The classifier scores (based on 6 gene assays) for the 69 patients used for qPCR validation were calculated, the scores rank, and the patients grouped into quarters. Kaplan Meier curves depict the overall survival for all quarters (from lowest to highest—red, blue, green, black) and demonstrate the association of the classifier with survival for all groups.

FIG. 7 shows concordant survival genes among 4 independent microarray studies in GBM. A composite index based on the average expression of the 38 concordant genes was calculated for each of the 110 GBM samples in the meta-analysis. The samples were ranked according to this index and divided into quartiles. Kaplan-Meier analysis indicates clear survival differences based on the expression of these 38 genes.

FIG. 8 shows Kaplan-Meier curves of metagene scores from TaqMan® QRT-PCR from formalin-fixed, paraffin embedded newly diagnosed GBM samples. A metagene score was calculated for each of 68 samples using a subset of 27 genes from the 38-gene list. Tumors were ranked by metagene score and separated by quartiles. The lowest quarter is compared with the upper 3 quarters and shows significantly (p<0.05) improved survival.

FIG. 9 shows an exemplary Phase I/II study adaptive randomization factorial design targeting mesenchymal/angiogenic phenotype and AKT pathway activation in glioblastoma, including in newly diagnosed glioblastoma.

FIG. 10 shows 38 exemplary genes associated with survival, their fold change, and their mesenchymal/angiogenic vs. proneural nature.

FIG. 11 illustrates validation of exemplary 14-Gene Predictor in temozolomide-radiation treated GBM.

FIG. 12 shows 57 exemplary genes found to be associated with survival in ¾ data sets. Genes present in the list of the top 200 survival genes are shown, listing the datasets in which each was present. The direction of the survival association (i.e. higher vs. lower expression in poor survivors) is shown.

FIG. 13 shows rank product analysis of microarray data. The 4 microarray datasets were subject to Rank Product analysis, as previously described. The top 100 genes from that analysis are shown, sorted by decreasing rank. Genes that overlap with the original 38-gene set as well as the 57 genes common to ¾ datasets are indicated.

### DESCRIPTION OF THE ILLUSTRATIVE EMBODIMENTS

### I. Definitions

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “about” means, in general, the stated value plus or minus 5%.

The use of the term “or” in the claims is used to mean “and/or” unless explicitly indicated to refer to alternatives only or the alternative are mutually exclusive, although the disclosure supports a definition that refers to only alternatives and “and/or.”

The term “good” as used herein may be referred to as “favorable.”

The term “good responder” as used herein refers to an individual whose tumor does not demonstrate growth, for example based on serial imaging studies, an individual that does not experience neurological decline attributable to the tumor over a period of about 1 year following initial diagnosis, and/or an individual that experiences a life span of about 2 years or more following initial diagnosis.

The term “housekeeping gene” as used herein refers to a gene involved in basic functions needed for maintenance of the cell. Housekeeping genes are transcribed at a relatively constant level and are thus used to normalize expression levels of genes that vary across different samples, for example. Examples include GAPDH, β-glucuronidase (GUSB), actin, ubiquitin, tubulin, and so forth.

The term “microarray” refers to an ordered arrangement of hybridizable array elements, preferably polynucleotide probes, on a substrate.

The term “poor” as used herein may be used interchangeably with “unfavorable.”

The term “poor responder” as used herein refers to an individual whose tumor grows during or shortly therafter standard therapy, for example radiation-chemotherapy, or who experiences a clinically evident neurologic decline attributable to the tumor.

The term “prognosis” as used herein refers to a forecast as to the probable outcome of cancer, including the prospect of recovery from the cancer.

The term “reference gene set” as used herein refers to one or more genes the expression of which is provided or obtained such that it can be compared to the expression of one or more of the genes listed in Table 4. In specific embodiments, the reference set comprises one or more housekeeping genes.

The term “respond to therapy” as used herein refers to an individual whose tumor either remains stable or becomes smaller during or shortly therafter standard therapy, for example radiation-chemotherapy.

The term “set” as used herein refers to two or more of a species, such as two or more genes, for example, or two or more reference RNA transcripts, for example.

### II. The Present Invention

Standard therapy benefits only a subset of individuals with newly diagnosed glioblastoma (GBM). Although several published studies have identified different gene expression profiles associated with outcome in glioblastoma, none have identified a consensus panel of biomarkers with robust predictive power to distinguish sensitive from refractory GBM tumors, for example.

In embodiments of the present invention, a meta-analysis was conducted comprising 110 GBM cases from 4 independent expression array datasets. To optimize identification of a robust consensus gene expression predictor, several statistical methods were tested for identifying genes associated with outcome. Initial validation was performed in an independent set of 69 GBM tumor samples. It was demonstrated that outcome prediction from gene expression data in GBM is feasible by showing that gene expression signatures derived from any 3 datasets (training set) could predict 2-year survival in the remaining dataset (test set). Identification of the top survival-associated genes common to all four datasets revealed a consensus 38-gene set. Better outcome was associated with increased expression of genes associated with neural development; poorer outcome was associated with increased expression of genes associated with mesenchymal differentiation, extracellular matrix, and angiogenesis. The multigene set was validated as a robust predictor of survival and radiation response in an independent set of samples. Therefore, a consensus gene expression profile was identified that is predictive of outcome in GBM with clinical application for the individualization of therapy. The mesenchymal/angiogenic signature common to refractory tumors indicates considerations for exploring different therapeutic approaches for individuals with aggressive tumors.

### III. Polynucleotides

Certain non-limiting but exemplary embodiments of the present invention concern nucleic acids, such as those whose level in a cell may be ascertained, those from a sample of a cell, those that would be utilized as probes for a microarray, and/or those that would be affixed to a microarray, for example. In certain aspects, both wild-type and mutant versions of these sequences will be employed. The term “nucleic acid” is well known in the art. A “nucleic acid” as used herein will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleotide bases in length. The term “polynucleotide” refers to at least one molecule of greater than about 100 nucleotide bases in length.

In certain embodiments, a “gene” refers to a nucleic acid that is transcribed. In certain aspects, the gene includes regulatory sequences involved in transcription or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered nucleic acid segments may express, or may be adapted to express proteins, polypeptides, polypeptide domains, peptides, fusion proteins, mutant polypeptides and/or the like.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid as originally isolated, and does not exclude genes or coding regions later added to the nucleic acid by the hand of man.

Polynucleotides of the invention may be envisioned to be those that hybridize to one of SEQ ID NO:1 through SEQ ID NO:38, or the complement thereof. As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244 each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

### IV. Polynucleotides of the Invention

In addition to the genes of Table 4, wherein exemplary sequences are provided as SEQ ID NOs:1-38, the invention also includes degenerate nucleic acids that include alternative codons to those present in the native materials. For example, serine residues are encoded by the codons TCA, AGT, TCC, TCG, TCT, and AGC. Each of the six codons is equivalent for the purposes of encoding a serine residue. Similarly, nucleotide sequence triplets that encode other amino acid residues include, but are not limited to: CCA, CCC, CCG, and CCT (proline codons); CGA, CGC, CGG, CGT, AGA, and AGG (arginine codons); ACA, ACC, ACO, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code, for example.

The invention also provides modified nucleic acid molecules, which include additions, substitutions, and deletions of one or more nucleotides such as the allelic variants and SNPs described above. In preferred embodiments, these modified nucleic acid molecules and/or the polypeptides they encode retain at least one activity or function of the unmodified nucleic acid molecule and/or the polypeptides, such as hybridization, antibody binding, etc. In certain embodiments, the modified nucleic acid molecules encode modified polypeptides, preferably polypeptides having conservative amino acid substitutions. As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. The modified nucleic acid molecules are structurally related to the unmodified nucleic acid molecules and in preferred embodiments are sufficiently structurally related to the unmodified nucleic acid molecules so that the modified and unmodified nucleic acid-molecules hybridize under stringent conditions known to one of skill in the art.

Polynucleotides of the invention include not only those that are provided in an exemplary manner as SEQ ID NOS:1-38, but polynucleotides that are about 70% to one of the provided sequences, about 75% identical to one of the provided sequences, about 80% identical to one of the provided sequences, about 85% identical to one of the provided sequences, about 90% identical to one of the provided sequences, about 95% identical to one of the provided sequences, about 97% identical to one of the provided sequences, or about 99% identical to one of the provided sequences. In additional embodiments, the polynucleotides comprise those that would hybridize under stringent conditions to a sequence of SEQ ID NOS:1-38 or the complement thereto.

For example, modified nucleic acid molecules that encode polypeptides having single amino acid changes can be prepared for use in the methods and products disclosed herein. Each of these nucleic acid molecules can have one, two, or three nucleotide substitutions is exclusive of nucleotide changes corresponding to the degeneracy of the genetic code as described herein Likewise, modified nucleic acid molecules that encode polypeptides having two amino acid changes can be prepared, which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions [e.g., by introduction of a stop codon or a splice site(s)] also can be prepared and are embraced by the invention as readily envisioned by one of ordinary skill in the art. Any of the foregoing nucleic acids can be tested by routine experimentation for retention of structural relation to or activity similar to the nucleic acids disclosed herein.

In the invention, standard hybridization techniques of microarray technology are utilized to assess patterns of nucleic acid expression and identify nucleic acid marker expression. Microarray technology, which is also known by other names including: DNA chip technology, gene chip technology, and solid-phase nucleic acid array technology, is well known to those of ordinary skill in the art and is based on, but not limited to, obtaining an array of identified nucleic acid probes an a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

According to the present invention, microarray substrates may include but are not limited to glass, silica, aluminosilicates, borosilicates, metal oxides such as alumia and nickel oxide, various clays, nitrocellulose, or nylon. In all embodiments a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes are sets of two or more of the nucleic acid molecules set forth as SEQ ID NO:1 though 38 (see also Table 4). Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, but are not limited to: amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photohenmical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium (Gwynne and Page. 2000). In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets are nucleic acids selected from the group, including but not limited to: DNA, genomic DNA, cDNA, RNA, mRNA and may be natural or synthetic. In all embodiments, nucleic acid molecules from human brain tissue are preferred. The tissue may be obtained from a subject or may be grown in culture (e.g. from a brain cancer cell line).

In embodiments of the invention one or more control nucleic acid molecules are attached to the substrate. Preferably, control nucleic acid molecules allow determination of factors including but not limited to nucleic acid quality and binding characteristics; reagent quality and effectiveness; hybridization success; and analysis thresholds and success. Control nucleic acids may include but are not limited to expression products of genes such as housekeeping genes or fragments thereof.

### V. Glioblastoma

Of primary brain tumors, glioblastoma multiforme (GBM) is the most common and most aggressive. According to the World Health Organization (WHO) classification of primary brain tumors, GBM is considered a grade IV astrocytoma. GBM is highly malignant, significantly infiltrates the brain, and may become extensive before becoming symptomatic.

GBM is an anaplastic, highly cellular tumor with poorly differentiated, round, or pleomorphic cells, occasional multinucleated cells, nuclear atypia, and anaplasia. According to the modified WHO classification, GBM differs from anaplastic astrocytomas (AA) by identification of necrosis microscopically. Variants of the tumor include at least gliosarcoma, multifocal GBM, or gliomatosis cerebri (in which the entire brain may be infiltrated with tumor cells). GBM infrequently metastasizes to the spinal cord or outside the nervous system.

Similar to other brain tumors, GBM produces symptoms by a combination of focal neurological deficits from compression and infiltration of the surrounding brain, vascular compromise, and raised intracranial pressure. Exemplary presenting symptoms may include at least one or more of the following: 1) headaches, which are nonspecific and indistinguishable from tension headache unless the tumor enlarges, in which case it may have features of increased intracranial pressure; 2) seizures, wherein depending on the tumor location, seizures may be simple partial, complex partial, or generalized; 3) focal neurological deficits, such as cognitive problems, neurological deficits resulting from radiation necrosis, communicating hydrocephalus, and in some cases cranial neuropathies and polyradiculopathies from leptomeningeal spread; 4) mental status changes, wherein personality changes may occur.

GBM tumors in less critical areas (e.g., anterior frontal or temporal lobe) may present with subtle personality changes and memory problems, and in tumors arising in the frontal or parietal lobes and thalamic regions, motor weakness and sensory hemineglect may present. Sensory neglect occurs more prominently in right hemispheric lesions. Seizures commonly presentation with small tumors in the frontoparietal regions (simple motor or sensory partial seizure) and temporal lobe (simple or complex partial seizure). Occipital lobe tumors may present with visual field defects. There is usually slow onset of a cortically based hemianopsia, and these tumors occur less frequently than tumors originating at other sites. Brainstem GBMs may be rare, but they may present with bilateral crossed neurological deficits (e.g., weakness on one side with contralateral cranial nerve palsy). In alternative cases, they may present with rapidly progressive headache or altered consciousness.

At least two genetic pathways have been associated with development of GBM: de novo (primary) glioblastomas, which are most common, and secondary glioblastomas. De novo GBM demonstrates a high rate of epidermal growth factor receptor (EGFR) overexpression, phosphatase and tensin homologue deleted on chromosome 10 (PTEN) mutations, and p16INK4A deletions. Secondary GBM often have TP53 and retinoblastoma gene (RB) mutations.

### VI. Gene Expression Profiling

Gene expression profiling may utilize measuring levels of nucleic acid, such as RNA, including mRNA, and/or protein. Methods of gene expression profiling include methods based on hybridization analysis of polynucleotides, methods based on sequencing of polynucleotides, and proteomics-based methods. The most commonly used methods known in the art for the quantification of mRNA expression in a sample include northern blotting and in situ hybridization (Parker & Barnes, Methods in Molecular Biology 106:247 283 (1999)); RNAse protection assays (Hod, Biotechniques 13:852 854 (1992)); and PCR-based methods, such as reverse transcription polymerase chain reaction (RT-PCR) (Weis et al., Trends in Genetics 8:263 264 (1992)), including quantitative RT-PCR. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

A. PCR-Based Gene Expression Profiling Methods

1. Reverse Transcriptase PCR (RT-PCR)

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including brain, breast, lung, colon, prostate, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure.™. Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700.™. Sequence Detection System.™. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700.™. Sequence Detection System.™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin, for example.

A more recent variation of the RT-PCR technique is the real time quantitative PCR, which measures PCR product accumulation through a dual-labeled fluorogenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996).

The steps of a representative protocol for profiling gene expression using fixed, paraffin-embedded tissues as the RNA source, including mRNA isolation, purification, primer extension and amplification are given in various published journal articles (for example: T. E. Godfrey et al. J. Molec. Diagnostics 2: 84 91 [2000]; K. Specht et al., Am. J. Pathol. 158: 419 29 [2001]). Briefly, a representative process starts with cutting about 10.mu.m thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

2. MassARRAY System

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

3. Other PCR-Based Methods

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray.™. technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)).

B. Microarrays

Differential gene expression can also be identified, or confirmed using the microarray technique. Thus, the expression profile of glioblastoma-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus, RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

C. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997).

D. Gene Expression Analysis by Massively Parallel Signature Sequencing (MPSS)

This method, described by Brenner et al., Nature Biotechnology 18:630 634 (2000), is a sequencing approach that combines non-gel-based signature sequencing with in vitro cloning of millions of templates on separate 5.mu.m diameter microbeads. First, a microbead library of DNA templates is constructed by in vitro cloning. This is followed by the assembly of a planar array of the template-containing microbeads in a flow cell at a high density (typically greater than 3.times.10.sup.6 microbeads/cm.sup.2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

E. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

F. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

G. General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles (for example: T. E. Godfrey et al., J Molec. Diagnostics 2: 84 91 [2000]; K. Specht et al., Am. J. Pathol. 158: 419 29 [2001]). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the individual on the basis of the characteristic gene expression pattern identified in the tumor sample examined, dependent on the predicted likelihood of cancer recurrence.

H. Glioblastoma Reference Set

An important aspect of the present invention is to use the measured expression of certain genes by cancer tissue to provide prognostic information. For this purpose it is necessary to correct for (normalize away) differences in the amount of RNA assayed and variability in the quality of the RNA used, for example. Therefore, the assay typically measures and incorporates the expression of certain normalizing genes, including well known housekeeping genes, such as GAPDH, GUSB, and Cyp1, for example. Alternatively, normalization can be based on the mean or median signal (Ct) of all of the assayed genes or a large subset thereof (global normalization approach). On a gene-by-gene basis, measured normalized amount of a patient tumor mRNA is compared to the amount found in a cancer tissue reference set. The number (N) of cancer tissues in this reference set should be sufficiently high to ensure that different reference sets (as a whole) behave essentially the same way. If this condition is met, the identity of the individual cancer tissues present in a particular set will have no significant impact on the relative amounts of the genes assayed. In specific embodiments, normalized expression levels for each mRNA/tested tumor/individual is expressed as a percentage of the expression level measured in the reference set. More specifically, the reference set of a sufficiently high number of tumors yields a distribution of normalized levels of each mRNA species. The level measured in a particular tumor sample to be analyzed falls at some percentile within this range, which can be determined by methods well known in the art. Below, unless noted otherwise, reference to expression levels of a gene assume normalized expression relative to the reference set although this is not always explicitly stated.

I. Exemplary Methods for Determining Expression Levels

According to the practice of the present invention, a sample from an individual is obtained. In specific embodiments, a sample of affected tissue is removed from a cancer patient, for example by conventional biopsy techniques that are well-known to those skilled in the art. The sample may be obtained from the individual prior to initiation of therapy, for example prior to onset of radiotherapy and/or chemotherapy. The sample may be prepared for a determination of expression level of one or more of the genes in Table 4, for example.

Determining the relative level of expression of the Table 4 genes in the tissue sample may comprise determining the relative number of RNA transcripts, particularly mRNA transcripts in the sample tissue and/or determining the relative level of the corresponding protein in the sample tissue. In specific embodiments, the relative level of protein in the sample tissue is determined by an immunoassay whereby an antibody that binds the corresponding protein is contacted with the sample tissue. The relative expression level in cells of the sampled tumor is conveniently determined with respect to one or more standards. The standards may comprise, for example, a relative expression level compared to a control gene in the sample, such as one or more housekeeping genes, a zero expression level on the one hand and the expression level of the gene in normal tissue of the same individual, or the expression level in the tissue of a normal control group on the other hand. The standard may also comprise the expression level in a standard cell line. The size of the change in expression in comparison to normal expression levels is indicative of the prognosis and/or response to therapy, in particular embodiments of the invention.

Methods of determining the level of mRNA transcripts of a particular gene in cells of a tissue of interest are well-known to those skilled in the art. According to one such method, total cellular RNA is purified from the affected cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters by, e.g., the so-called “Northern” blotting technique. The RNA is immobilized on the filters by heating. Detection and quantification of specific RNA is accomplished using appropriately labelled DNA or RNA probes complementary to the RNA in question. See Molecular Cloning: A Laboratory Manual, J. Sambrook et al., eds., 2nd edition, Cold Spring Harbor Laboratory Press, 1989, Chapter 7, the disclosure of which is incorporated by reference.

In addition to blotting techniques, the mRNA assay test may be carried out according to the technique of in situ hybridization. The latter technique requires fewer tumor cells than the Northern blotting technique. Also known as “cytological hybridization”, the in situ technique involves depositing whole cells onto a microscope cover slip and probing the nucleic acid content of the cell with a solution containing radioactive or otherwise labelled cDNA or cRNA probes. The practice of the in situ hybridization technique is described in more detail in U.S. Pat. No. 5,427,916, for example, the entire disclosure of which is incorporated herein by reference.

The nucleic acid probes for the above RNA hybridization methods can be designed based upon sequences provided in the National Center for Biotechnology Information's GenBank® database.

Either method of RNA hybridization, blot hybridization or in situ hybridization, can provide a quantitative result for the presence of the target RNA transcript in the RNA donor cells. Methods for preparation of labeled DNA and RNA probes, and the conditions for hybridization thereof to target nucleotide sequences, are described in Molecular Cloning, supra, Chapters 10 and 11, incorporated herein by reference.

The nucleic acid probe may be labeled with, e.g., a radionuclide such as ³²P, ¹⁴C, or ³⁵S; a heavy metal; or a ligand capable of functioning as a specific binding pair member for a labelled ligand, such as a labelled antibody, a fluorescent molecule, a chemolescent molecule, an enzyme or the like.

Probes may be labelled to high specific activity by either the nick translation method or Rigby et al., J. Mol. Biol. 113: 237-251 (1977) or by the random priming method, Fienberg et al., Anal. Biochem. 132: 6-13 (1983). The latter is the method of choice for synthesizing ³²P-labelled probes of high specific activity from single-stranded DNA or from RNA templates. Both methods are well-known to those skilled in the art and will not be repeated herein. By replacing preexisting nucleotides with highly radioactive nucleotides, it is possible to prepare ³²P-labelled DNA probes with a specific activity well in excess of 10⁸ cpm/microgram according to the nick translation method. Autoradiographic detection of hybridization may then be performed by exposing filters on photographic film. Densitometric scanning of the filters provides an accurate measurement of mRNA transcripts.

Where radionuclide labelling is not practical, the random-primer method may be used to incorporate the dTTP analogue 5-(N—(N-biotinyl-epsilon-aminocaproyl)-3-aminoallyl)deoxyuridine triphosphate into the probe molecule. The thus biotinylated probe oligonucleotide can be detected by reaction with biotin binding proteins such as avidin, streptavidin, or anti-biotin antibodies coupled with fluorescent dyes or enzymes producing color reactions.

The relative number of transcripts may also be determined by reverse transcription of mRNA followed by amplification in a polymerase chain reaction (RT-PCR), and comparison with a standard. The methods for RT-PCR and variations thereon are well known to those of ordinary skill in the art.

According to another embodiment of the invention, the level of gene expression in cells of the individual's tissue is determined by assaying the amount of the corresponding protein. A variety of methods for measuring expression of the protein exist, including Western blotting and immunohistochemical staining. Western blots are run by spreading a protein sample on a gel, using an SDS gel, blotting the gel with a cellulose nitrate filter, and probing the filters with labeled antibodies. With immunohistochemical staining techniques, a cell sample is prepared, typically by dehydration and fixation, followed by reaction with labeled antibodies specific for the gene product coupled, where the labels are usually visually detectable, such as enzymatic labels, florescent labels, luminescent labels, and the like.

According to one embodiment of the invention, tissue samples are obtained from individuals and the samples are embedded then cut to e.g. 3-5 μm, fixed, mounted and dried according to conventional tissue mounting techniques. The fixing agent may advantageously comprise formalin. The embedding agent for mounting the specimen may comprise, e.g., paraffin. The samples may be stored in this condition. Following deparaffinization and rehydration, the samples are contacted with an immunoreagent comprising an antibody specific for the protein. The antibody may comprise a polyclonal or monoclonal antibody. The antibody may comprise an intact antibody, or fragments thereof capable of specifically binding the protein. Such fragments include, but are not limited to, Fab and F(ab′)₂ fragments. As used herein, the term “antibody” includes both polyclonal and monoclonal antibodies. The term “antibody” means not only intact antibody molecules, but also includes fragments thereof which retain antigen binding ability.

Appropriate polyclonal antisera may be prepared by immunizing appropriate host animals with protein and collecting and purifying the antisera according to conventional techniques known to those skilled in the art. Monoclonal antibody may be prepared by following the classical technique of Kohler and Milstein, Nature 254:493-497 (1975), as further elaborated in later works such as Monoclonal Antibodies, Hybridomas: A New Dimension in Biological Analysis, R. H. Kennet et al., eds., Plenum Press, New York and London (1980).

Substantially pure protein for use as an immunogen for raising polyclonal or monoclonal antibodies may be conveniently prepared by recombinant DNA methods. According to one such method, protein is prepared in the form of a bacterially expressed glutathione S-transferase (GST) fusion protein. Such fusion proteins may be prepared using commercially available expression systems, following standard expression protocols, e.g., “Expression and Purification of Glutathione-S-Transferase Fusion Proteins”, Supplement 10, unit 16.7, in Current Protocols in Molecular Biology (1990). Also see Smith and Johnson, Gene 67: 34-40 (1988); Frangioni and Neel, Anal. Biochem. 210: 179-187 (1993). Briefly, DNA encoding for the protein is subcloned into an appropriate vector in the correct reading frame and introduced into E. coli cells. Transformants are selected on LB/ampicillin plates; the plates are incubated 12 to 15 hours at 37° C. Transformants are grown in isopropyl-β-D-thiogalactoside to induce expression of GST fusion protein. The cells are harvested from the liquid cultures by centrifugation. The bacterial pellet is resuspended and the cell pellet sonicated to lyse the cells. The lysate is then contacted with glutathione-agarose beads. The beads are collected by centrifugation and the fusion protein eluted. The GST carrier is then removed by treatment of the fusion protein with thrombin cleavage buffer. The released protein is recovered.

As an alternative to immunization with the complete protein molecule, antibody against the protein can be raised by immunizing appropriate hosts with immunogenic fragments of the whole protein, particularly peptides corresponding to the carboxy terminus of the molecule.

The antibody either directly or indirectly bears a detectable label. The detectable label may be attached to the primary anti-protein antibody directly. More conveniently, the detectable label is attached to a secondary antibody, e.g., goat anti-rabbit IgG, which binds the primary antibody. The label may advantageously comprise, for example, a radionuclide in the case of a radioimmunoassay; a fluorescent moiety in the case of an immunofluorescent assay; a chemiluminescent moiety in the case of a chemiluminescent assay; or an enzyme which cleaves a chromogenic substrate, in the case of an enzyme-linked immunosorbent assay.

Most preferably, the detectable label comprises an avidin-biotin-peroxidase complex (ABC) which has surplus biotin-binding capacity. The secondary antibody is biotinylated. To locate the antigen in the tissue section under analysis, the section is treated with primary antiserum against the protein, washed, and then treated with the secondary antiserum. The subsequent addition of ABC localizes peroxidase at the site of the specific antigen, since the ABC adheres non-specifically to biotin. Peroxidase (and hence antigen) is detected by incubating the section with e.g. H₂O₂ and diaminobenzidine (which results in the antigenic site being stained brown) or H₂O₂ and 4-chloro-1-naphthol (resulting in a blue stain).

The ABC method can be used for paraffin-embedded sections, frozen sections, and smears. Endogenous (tissue or cell) peroxidase may be quenched e.g. with H₂O₂ in methanol.

The level of protein expression in tumor samples may be compared on a relative basis to the expression in normal tissue samples by comparing the stain intensities, or comparing the number of stained cells. The lower the stain intensity with respect to the normal controls, or the lower the stained cell count in a tissue section having approximately the same number of cells as the control section, the lower the expression of the gene, and hence the higher the expected malignant potential of the sample.

### VII. Determination of Prognosis and Therapy Responders

In the multigene predictor embodiments, some of the genes are overexpressed in the poor survivors and underexpressed in good survivors, and these genes may be considered deleterious for glioblastoma. In other embodiments, there are also genes that are underexpressed in the poor survivors and overexpressed in good survivors, and these genes may be considered beneficial for glioblastoma. In certain aspects, an individual that has a tumor that has either high expression of the deleterious genes and/or low expression of beneficial genes would be expected to do poorly. To condense the multigene set for a given tumor sample into a single number, the simple following exemplary formula may be utilized, in certain embodiments:

(bad gene1+bad gene2+bad gene3,etc.)−(good gene1+good gene2+good gene3,etc.)=“metagene” score.

A reference set of tumors is employed for comparison. In specific embodiments, a set of GBMs (for example, 100) from patients who have been treated with standard therapy with known outcome may be employed. In specific aspects, about 25% will live 2 years, and the reference set is representative of GBM as a whole.

Metagene scores are calculated in this reference set, and they are ranked. A score that is in the upper 75th percentile relative to this ranked set of reference tumors is considered predictive of poor survival, while scores in the lowest 25th percentile are considered predictive of better survival, in particular embodiments.

Such metagene score comparisons may be employed to determine a prognosis for an individual with glioblastoma and/or may be employed to determine whether or not an individual will respond to therapy.

### VIII. Exemplary Genes Associated with Survival and/or Therapy Prediction in Glioblastoma

The following exemplary genes are associated with survival and/or therapy prediction in glioblastoma: TIMP1, YKL-40, IGFBP2, LGALS3, LGALS1, KIAA0509, AQP1, RTN1, LDHA, GRIA2, EMP3, FABP5, GABBR1, TNC, COL1A2, OLIG2, VEGF, MAOB, FN1, SERPINA3, PDPN, TAGLN, NNMT, CLIC1, SERPING1, IGFBP3, SERPINE1, TMSB10, TGFBI, GPNMB, TCTE1L, RIS1, TAGLN2, ACTN1, TCF12, PLP2, OMG, and S100A10. In some cases, expression of one or more of these genes is increased in individuals that have good prognosis and/or will respond to therapy. In other cases, expression of one or more of these genes is decreased in individuals that have good prognosis and/or will respond to therapy. In other cases, expression of one or more of these genes is increased in individuals that have poor prognosis and/or will not respond to therapy. In still other cases, expression of one or more of these genes is decreased in individuals that have poor prognosis and/or will not respond to therapy.

In specific cases, the expression level of one or more genes listed in Table 4 is determined, wherein increased expression of one or more of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10 indicates poor prognosis and/or therapy response and therefore a decreased likelihood of long-term survival without cancer recurrence and/or wherein decreased expression of one or more of KIAA0509, RTN1, GRIA1, GABBR1, OLIG2, TCF12, and OMG indicates good prognosis and/or good therapy response and therefore an increased likelihood of long-term survival without cancer recurrence.

In a different embodiment, the invention concerns a combined RT-PCR test involving one or more of the following genes: TIMP1, CHI3L1, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFBI, GPNMB, TCTE1L, RIS1, TAGLN2, ACTN1, PLP2, PBEF, LTF1, CHI3L2, SEC61G, DKFZp564K0822, EGFR, and S100A10, whose elevated expression levels indicate poor prognosis and/or poor response to therapy; as well as one or more of the following genes: KIAA0509, RTN1, GRIA2, GABBR1, OLIG2, TCF12, OMG, C10orf56, ID1, PDGFRA, and C1QL1, whose elevated expression levels indicate good prognosis and/or good response to therapy.

In specific embodiments of the invention, prognostic and/or therapeutic information for the prediction of patient outcome is obtained from expression levels of one or more of the following: PDPN, AQP1, YKL40, GPNMB, EMP3, S100, IGFBP2, LGALS3, SERPE3, TNC, NNMT, VEGFA, TCTEIL, MAOB, TAGLN2, RTN1, KIAA0510, OLIG2, GABA, EGFR, CHI3L2, C1QL1, PDGFRA, ID1, and LTF.

### IX. Samples from the Individual

A sample from the individual is obtained, such as, for example, one that comprises one or more glioblastoma cells or cells that are suspected of being glioblastoma cells. In specific embodiments, the sample is obtained by any suitable means in the art, for example, by biopsy. The sample may comprise one or more brain cells, in specific embodiments. The sample may comprise nucleic acid and/or protein.

A sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the nucleic acid and/or protein for use in the invention are well known to one of ordinary skill in the art. An example of this, although not intended to be limiting, is that in some instances a sample from the biopsy may be sufficient for assessment of RNA expression without amplification, but in other instances the lack of suitable cells in a small biopsy region may require use of RNA conversion and/or amplification methods or other methods to enhance resolution of the nucleic acid molecules. Such methods, which allow use of limited biopsy materials, are well known to those of ordinary skill in the art and include, but are not limited to, direct RNA amplification, reverse transcription of RNA to cDNA, amplification of cDNA, or the generation of radio-labeled nucleic acids.

Determining the expression of a set of nucleic acid molecules in the brain tissue comprises identifying RNA transcripts in the tissue sample by analysis of nucleic acid and/or protein expression in the tissue sample. As used herein, “set” refers to a group of nucleic acid molecules that include 2, 3, 4, 5, 6, 7, 8, 9, 10, 11, 12, 13, 14, 15, 16 17, 18, 19, 20, 21, 22, 23, 24, 25, 26, 27, 28, 29, 30, 31, 32, 33, 34, 35, 36, 37, or 38 different nucleic acid sequences from the group of nucleic acid sequences numbered 1 through 38 in Table 4.

### X. Kits

Kits of the invention may comprise any suitable reagents to practice at least part of a method of the invention, and the kit and reagents are housed in one or more suitable containers. For example, the kit may comprise an apparatus for obtaining a sample from an individual, such as a needle, syringe, and/or scalpel, for example. The kit may comprise one or more polynucleotides of one or more of the genes listed in Table 4. In specific embodiments, the kit comprises one or more primers for amplication of one or more of the genes listed in Table 4.

Other reagents may include those suitable for polymerase chain reaction, such as nucleotides, thermophilic polymerase, buffer, and/or salt, for example.

The kit may comprise a substrate comprising polynucleotides, such as a microarray, wherein the microarray comprises one or more genes listed in Table 4 and no more than 5 housekeeping genes, but in specific cases no other genes are provided thereon. In specific aspects, the microarray comprises a representative sequence that is less than the full length sequence of the genes, so long as the representative sequence clearly signifies the corresponding gene.

### XI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods

Exemplary materials and methods may be utilized as follows.

### Gene Expression Array Datasets

The meta-analysis was performed using 4 previously published GBM microarray datasets (Nigro et al., 2005; Phillips et al., 2006; Freije et al., 2004; Nutt et al., 2003). Only World Health Organization-defined GBMs were included. The platform for all 4 datasets was Affymetrix-based and used 2 different chip types: U95Av2 and U133A. Data between these 2 chips were merged by mapping available probe sequence data with 2 databases (Pruitt et al., 2003; Imanishi et al., 2004).

### Identification of Gene Expression Profiles Associated With Survival

Cases were dichotomized into typical (<2 years) versus long-term (>2 years) survival groups (TS versus LTS, respectively). Several statistical approaches were investigated to identify genes with the highest association with survival including fold-change (ratio of mean expression between TS and LTS) and Significance Analysis of Microarrays (SAM) (Tusher et al., 2001). T-test p-value and Rank Product analysis (Breitling et al., 2004; Breitling and Herzyk, 2005) were also examined. Genes were ranked according to degree of difference between TS and LTS groups. The absolute value of this difference was used to allow identification of genes differentially expressed in either direction (e.g. higher expression in either TS or LTS).

Quantitative RT-PCR Measurement of Gene Expression from Paraffin-Embedded Tissue

Quantitative measurement of expression of candidate survival genes from formalin-fixed, paraffin embedded (FFPE) GBM samples were performed using TaqMan quantitative reverse transcriptase-polymerase chain reaction (qRT-PCR) assays. None of the samples used in this validation were the same as those used in the microarray meta-analysis.

### Gene Expression Array Data Sets

The meta-analysis was based on Affymetrix gene expression array data derived from frozen samples of newly diagnosed GBM tumors from four independent data sets from individual institutions. Two of these datasets, from the University of California-San Francisco (UCSF) and the University of Texas-MD Anderson Cancer Center (MDA) (Nigro et al., 2005; Phillips et al., 2006). Publicly available Affymetrix GeneChip data (.cel files) were obtained for data sets from the University of California-Los Angeles (UCLA) (Freije et al., 2004) and Massachusetts General Hospital (MGH) (Nutt et al., 2003). The current analysis only included data from newly diagnosed GBMs with clinical follow-up data sufficient to evaluate for 2-year-survival (either deceased or alive for at least 2 years of follow-up). Samples from patients known to have a prior neurosurgical procedure were excluded.

### Mapping Data Between Two Array Platforms

Because the data sets studied here involved two different platforms of microarrays (U95Av2 and U133A), extra caution was taken to map the data between the platforms. Although both platforms were developed by Affymetrix using photoliography, the selection of probe sequences followed different algorithms so that there is little overlap between the probe sets used. For the mapping, a database of full length mRNA transcripts was constructed by merging two publicly available databases: RefSeq (Pruitt et al., 2003) and H-InvDB (Imanishi et al., 2004). BLAST searches were performed for each of the probes used in the arrays against the database. Each matched target list was obtained from a BLAST search of a probe sequence against the library of full-length transcripts with the option of filtering the repetitive and low composite sequences turned off. New probe sets were defined by grouping probes that share the same matched target lists. Only exact matches covering the full-length of a probe were collected in the matched target lists. The mapping enhances the reproducibility between the two microarray platforms because it ensures that the matching probesets on the two platforms target the same genes.

### Data Normalization and Sample Quality Control

Probe sets were mapped from the U133A and U95Av2 based on matches to full length mRNA sequences to generate a single output with genes present on both platforms, as described above. The probe signals belonging to the common probe sets were normalized using quantile normalization for each sample from every institution so that the distributions of signals on an array were the same within a platform. Log-expression values were then extracted using the PDNN model (Zhang et al., 2003). The log expression values of probe sets were normalized using quantile normalization so that the distributions of log-expression on each array were the same. Because the PDNN algorithm has a tendency to compress the fold changes (Zhang et al., 2003) the log-expression values were rescaled by multiplying a factor of 2 based on prior comparisons of PDNN-extracted expression values and matched PCR measurements. Finally, the median value within each institution for each probe set was calculated and the measurements were expressed as median ratios within that institution. The last step was found to be critical for eliminating institutional bias in the gene expression data.

Recognizing that inclusion of surrounding non-neoplastic brain tissue would have a confounding effect on the results and interpretation of the expression profiling data, the inventors sought to eliminate samples with an apparent non-neoplastic brain “contamination”. A set of five genes (gamma-aminobutyric acid receptor 5 (GABRA5), neurogranin, somatostatin, synaptotagmin I, and the light polypeptide of neurofilament protein) were first identified that were found to be highly overexpressed in non-neoplastic brain relative to malignant glioma samples using a previously published data set (Nigro et al., 2005). A total of 146 cases from the four institutions fit the criteria of newly diagnosed GBM with sufficient follow-up to determine survival at 2 years. For each of the original 146 samples a “normal brain expression index” was calculated by averaging the expression levels of these five genes. Thirty-six cases exhibited a twofold or greater normal brain expression index of relative to the median, indicating probable “contamination” of the tumor sample by excessive normal brain tissue, and these samples were excluded from subsequent analysis. The number of cases from each of the 4 institutions represented in this set of 36 samples were as follows: UCLA: 18 cases; UCSF: 7 cases; MDA: 8 cases; MGH: 3 cases. Removal of the normal brain contaminated cases left 110 tumors for analysis and a summary of the clinical information of these cases are shown in Table 1.

TABLE 1
Exemplary Clinical and Microarray Platform Characteristics

| | Institution | | | |
|---|---|---|---|---|
| | MDA | MGH | UCLA | UCSF |
| Microarray Type | U133A | U95A | U133A | U95A |
| # of Samples | 32 | 24 | 27 | 27 |
| Typical Survivors (<2 yrs) | 20 | 17 | 19 | 21 |
| Long-Term Survivors (≧2 yrs) | 12 | 7 | 8 | 6 |

### Statistical Method and Concordance of Survival Association Across Institutions

It was reasoned that the method that resulted in the most consistent ranking of genes across institutions, and which performed best in cross-validation analyses, was most likely to identify a consensus gene expression profile predictive of survival in GBM.

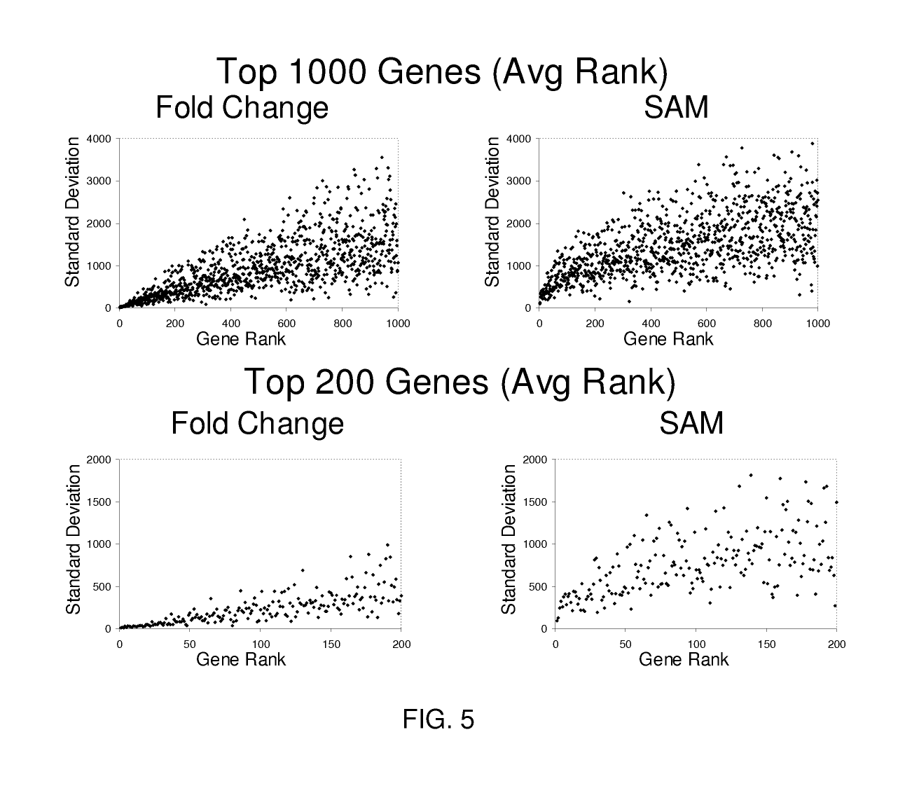

Both fold-change and SAM 2-class analysis were applied to each of the 4 institutional data sets (MGH, MDA, UCLA and UCSF) independently, and genes were ranked from the largest (or most significant) to smallest (or least significant) difference between TS and LTS groups for each statistical method. The standard deviation of the ranks across the 4 institutions for each gene was calculated and plotted against the average rank of each gene for each statistical method (FIG. 5). This analysis demonstrated that, in general, the most highly ranked genes showed the lowest standard deviations. It was also noted that the consistency of rankings (as measured by the magnitude of the average standard deviation) was continuous as a function of the average rank, but decreased substantially after the top 200 genes (FIG. 5). It is this relationship that indicated the choice of the top 200 genes within each institution as a threshold for the subsequent analyses. Overall, gene rankings by fold-change resulted in lower standard deviations as a function of rank than when SAM p-value was used (FIG. 5). These observations are consistent with recent results from the Microarray Quality Control (MAQC) Project demonstrating that fold-change was superior to p-value based significance approaches (SAM, t-test) in identifying concordance across studies due to the relatively unstable nature of the variance estimate in the t-statistic (Shi et al., 2006). Based on these considerations, fold-change was therefore used for subsequent analyses.

### Calculation of a Metagene Score

In order to determine the association of the overall gene expression classifier with patient outcome, a single “metagene” score was calculated for each case based on the set of 38 genes by summing the normalized expression values for all the genes associated with poor prognosis (n=31) and then subtracting the sum of the normalized expression values for all the genes associated with good prognosis (n=7) for each case. This resulted in a single numerical score for each tumor, and each tumor was then ranked according to this metagene score.

### False Discovery Rate of 38-Gene Concordant Set

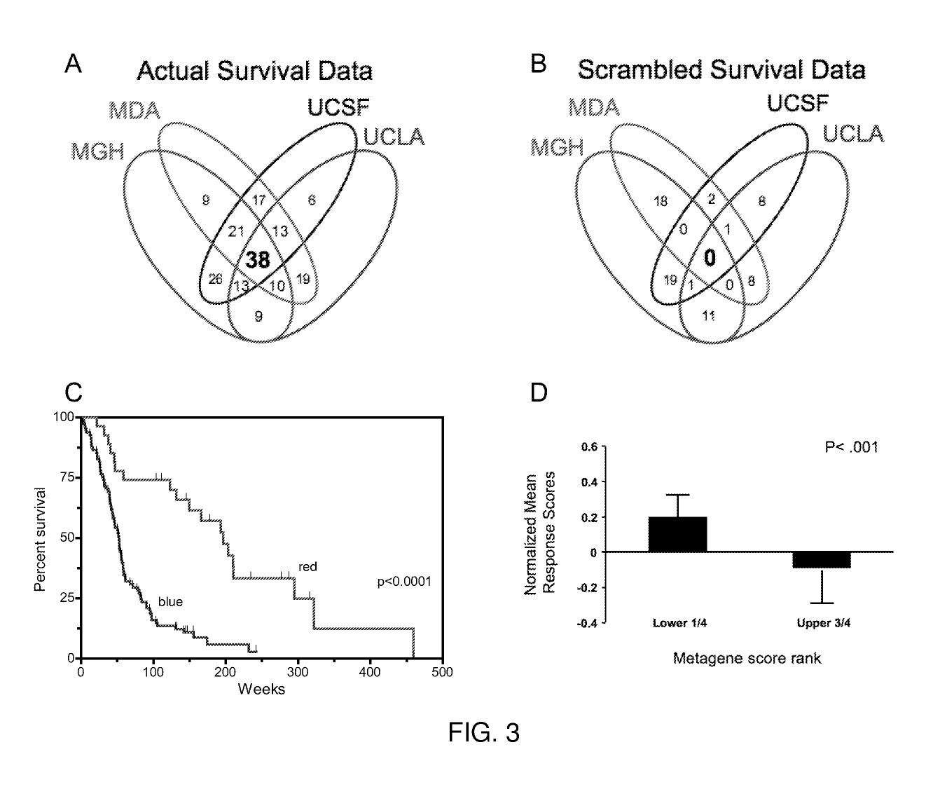

To determine whether these observed overlaps of 38 genes across 4 institutions was greater than those expected by chance, the survival times were scrambled and randomly assigned to individual cases, and the same analysis was performed. This analysis was repeated 5 times for graphical representation, and a representative example is shown in FIG. 3B. The expected false discovery rates were calculated for the identification of genes common to 4 out of 4 datasets using this approach and found that that there is a 0.3% chance to find 1 common gene among the four lists by chance, and a 99.7% chance that 0 genes would be common to the 4 lists by chance. Thus, the identification of a set of 38 genes associated with survival common to all 4 institutional datasets was highly unlikely to have occurred by chance.

Quantitative RT-PCR Measurement of Gene Expression from Paraffin Embedded Tissue

In order to optimize amplification of the fragmented RNA found in FFPE processed tissue, primers were designed with predicted amplicon sizes of 75 base pairs or less (Applied Biosystems, Foster City, Calif.; and Roche Applied Sciences, Indianapolis, Ind.) (Table 2). In Table 2, primers/probes used for real-time quantitative RT-PCR for FFPE GBM samples. GenBank® sequences are incorporated by reference herein in their entirety. Reagents were purchased either through the ABI “assay on demand” program (where the sequence is proprietary) or through Roche. When purchased from Roche, the primer sequence is indicated along with the probe #. Genes tested include the 38 genes identified in the microarray analysis plus 2 control genes GAPDH and GUSB).

TABLE 2

Primers/probes used for real-time quantitative RT-PCR for exemplary FFPE GBM
samples (see Legend for SEQ ID NOS for primers)

| | | | Roche | | |
|---|---|---|---|---|---|
| Gene | | | Universal | | Reverse |
| Symbol | accession # | ABI catalog # | Probe # | Forward primer sequence | primer sequence |
| AQP1 | NM_198098.1 | Hs00166067_m1 | | | |
| CHI3L1 | NM_001276.1 | Hs01072228_m1 | | | |
| COL1A2 | NM_000089.3 | Hs00164099_m1 | | | |
| GABBR1 | NM_001470.1 | Hs00559488_m1 | | | |
| GRIA2 | NM_000826.1 | Hs00181331_m1 | | | |
| GUSB | NM_000181.2 | Hs99999908_m1 | | | |
| IGFBP2 | NM_000597.1 | Hs00167151_m1 | | | |
| IGFBP3 | NM_000598.3 | Hs00426287_m1 | | | |
| LGALS1 | NM_002305.2 | Hs00169327_m1 | | | |
| LGALS3 | NM_002306.1 | Hs00173587_m1 | | | |
| NNMT | NM_006169.1 | Hs00196287_m1 | | | |
| OLIG2 | NM_005806.1 | Hs00377820_m1 | | | |
| RIS1 | NM_015444.1 | Hs00374916_sl | | | |
| RTN1 | NM_021136.2 | Hs00382515_m1 | | | |
| TIMP1 | NM_003254.1 | Hs00171558_m1 | | | |
| TNC | NM_002160.1 | Hs00233648_m1 | | | |
| ACTN1 | NM_001102.2 | | 42 | TGGCAGAGAAGTACCTGGACA | GGCAGTTCCAACGATGTCTT |
| CLIC1 | NM_001288.4 | | 16 | GACACCAACAAGATTGAGGAATT | GCCAGCTTGGGGTACCTG |
| EMP3 | NM_001425.1 | | 78 | GAGCGAGGGACAAGACTCC | GACATGGCTGCAGTGGAAG |
| FABP5 | NM_001444.1 | | 22 | CAAGAAAATTGAAAGATGGGAAA | CCGAGTACAGGTGACATTGTTC |
| FN1 | NM_002026.2 | | 64 | GCCACTGGAGTCTTTACCACA | CCTCGGTGTTGTAAGGTGGA |
| GAPDH | NM_002046.1 | | 9 | GGGAAGCTTGTCATCAATGG | TTGATTTTGGAGGGATCTCG |
| GPNMB | NM_001005340.1 | | 61 | TGCAAGATTGCCACTTGATG | CCCTCATGTAAGCAGAAGGTCT |
| LDHA | NM_005566.1 | | 47 | GTCCTTGGGGAACATGGAG | GACACCAGCAACATTCATTCC |
| MAOB | NM_000898.3 | | 60 | GAGAGAGCAGCCCGAGAG | GACTGCCAGATTTCATCCTC |
| OMG | NM_002544.3 | | 13 | ACGACACCACGGCTTTGATGG | CCAGGTGTGAGAAACAGAAGG |
| PDPN | NM_001006624.1 | | 20 | GGGTCCTGGCAGAAGGAG | CGCCTTCCAAACCTGTAGTC |
| PLP2 | NM_002668.1 | | 81 | GACCTGCACACCAAGATACC | CGCTATGAGGGTTCGGAAG |
| S100A10 | NM_002966.1 | | 76 | AGTTCCCTGGATTTTTGG | TGGTCCAGGTCCTTCAT |
| SERPINA3 | NM_001085.3 | | 14 | TCACAGGGGCCAGGAACCTA | TGCCCTCCTCAAATACATCAAG |
| SERPINE1 | NM_000602.1 | | 19 | AAGGCACCTCTGAGAACTTCA | CCCAGGACTAGGCAGGTG |
| SERPING1 | NM_000062.1 | | 20 | GACCCTGCTGACCCTCCT | GGAGCTGGTAGCATTTGGAT |
| TAGLN | NM_001001522.1 | | 2 | GGCCAAGGCTCTACTGTCTG | CCATGTCTGGGGAAAGCTC |
| TAGLN2 | NM_003564.1 | | 83 | CCAGCCCGCTTGAAC | CAGGCCATATGCAGGTC |
| TCF12 | NM_003205.3 | | 64 | CCCTGTACAGCAGAGATACTGGAT | AAGCCCCAGATCTTGTCTCA |
| TCTEIL | NM_006520.1 | | 76 | CAGAAGAGCGCATATGGCTT | CTTACGGTACAGGTTCCATC |
| TGFB1 | NM_000358.1 | | 5 | CTTCAAGCATCGTGTTGAGC | GACACCTTTGAGACCCTTCG |
| TMSB10 | NM_021103.2 | | 2 | CTGCCGACCAAAGAGACC | GGGTAGGAAATCCTCCAGG |
| TNR | AB007979.1 | | 6 | GACGATGCACACTTTAATTAGC | GAAGTTGGTTTTTCCTCTCC |
| VEGFA | NM_001025366.1 | | 9 | AGTGTGTGCCCACTGAGGA | GGTGAGGTTTGATCCGCATA |

Legend for Table 2

| | SEQ | | SEQ |
|---|---|---|---|
| | ID | | ID |
| Forward Primer Sequence | NO | Reverse Primer Sequence | NO |
| TGGCAGAGAAGTACCTGGACA | 39 | GGCAGTTCCAACGATGTCTT | 62 |
| GACACCAACAAGATTGAGGAATT | 40 | GCCAGCTTGGGGTACCTG | 63 |
| GAGCGAGGGACAAGACTCC | 41 | GACATGGCTGCAGTGGAAG | 64 |
| CAAGAAAATTGAAAGATGGGAAA | 42 | CCGAGTACAGGTGACATTGTTC | 65 |
| GCCACTGGAGTCTTTACCACA | 43 | CCTCGGTGTTGTAAGGTGGA | 66 |
| GGGAAGCTTGTCATCAATGG | 44 | TTGATTTTGGAGGGATCTCG | 67 |
| TGCAAGATTGCCACTTGATG | 45 | CCCTCATGTAAGCAGAAGGTCT | 68 |
| GTCCTTGGGGAACATGGAG | 46 | GACACCAGCAACATTCATTCC | 69 |
| GAGAGAGCAGCCCGAGAG | 47 | GACTGCCAGATTTCATCCTC | 70 |
| ACGACACCACGGCTTTGATGG | 48 | CCAGGTGTGAGAAACAGAAGG | 71 |
| GGGTCCTGGCAGAAGGAG | 49 | CGCCTTCCAAACCTGTAGTC | 72 |
| GACCTGCACACCAAGATACC | 50 | CGCTATGAGGGTTCGGAAG | 73 |
| AGTTCCCTGGATTTTTGG | 51 | TGGTCCAGGTCCTTCAT | 74 |
| TCACAGGGGCCAGGAACCTA | 52 | TGCCCTCCTCAAATACATCAAG | 75 |
| AAGGCACCTCTGAGAACTTCA | 53 | CCCAGGACTAGGCAGGTG | 76 |
| GACCCTGCTGACCCTCCT | 54 | GGAGCTGGTAGCATTTGGAT | 77 |
| GGCCAAGGCTCTACTGTCTG | 55 | CCATGTCTGGGGAAAGCTC | 78 |
| CCAGCCCGCTTGAAC | 56 | CAGGCCATATGCAGGTC | 79 |
| CCCTGTACAGCAGAGATACTGGAT | 57 | AAGCCCCAGATCTTGTCTCA | 80 |
| CAGAAGAGCGCATATGGCTT | 58 | CTTACGGTACAGGTTCCATC | 81 |
| CTTCAAGCATCGTGTTGAGC | 59 | GACACCTTTGAGACCCTTCG | 82 |
| CTGCCGACCAAAGAGACC | 60 | GGGTAGGAAATCCTCCAGG | 83 |
| GACGATGCACACTTTAATTAGC | 61 | GAAGTTGGTTTTTCCTCTCC | 84 |
| AGTGTGTGCCCACTGAGGA | 85 | GGTGAGGTTTGATCCGCATA | 86 |

QRT-PCR measurements were performed using a separate set of 69 FFPE GBM samples from the UT MD Anderson Brain Tumor Tissue Bank. The use of the tissue and clinical data for these studies were covered under a protocol approved by the MD Anderson IRB. Samples were examined and dissected if necessary by a neuropathologist (KA) to ensure purity of tumor tissue. RNA was isolated from these samples (Epicentre Biotechnologies, Madison, Wis.) following deparaffinization and proteinase K treatment. Total tumor RNA was reverse transcribed to single-stranded cDNA using ABI's High Capacity cDNA Archive kit (cat#4368814) using the maximum allowed concentration of total RNA per manufacturer's instructions (100 ng/μl). To determine fold-changes in each gene, qRT-PCR was performed on a Chromo4™ Real-Time PCR Detector from Bio-Rad (Hercules, Calif.) using the primers and probes shown in Table 2. In triplicate, 1 μl cDNA was amplified for each sample for each assay in a reaction containing 1× TaqMan® Universal PCR Master Mix without AmpErase UNG and 1× gene expression assay with the following cycling conditions: 10 minutes at 95° C., then 40 cycles of 95° C. for 15 seconds and 60° C. for 1 minute. The ΔCt values for each gene were calculated by comparison with the average of the Ct values for 2 control genes (GAPDH, GUSB) for each tumor case. To determine the survival association for each gene, the mean ΔCt for the typical survivor (TS) cases was compared with that of the long-term survivor (LTS) cases, and the ΔΔCt representing the difference of these means (TS minus LTS) was determined. Fold-change associated with survival for each gene was determined by raising 2 to the power of the ΔΔCt and taking the reciprocal of this value. Since with qRT-PCR data, a more negative value indicates higher expression, the signs of the ΔCt values were reversed to be consistent with the Affymetrix level (i.e. higher metagene score would predict worse outcome).

Optimization of Survival Genes from qRT-PCR Data

Methods to identify optimal gene lists to identify the optimal multigene predictor from microarray data or qRT-PCR data are not well established. Examination of the qRT-PCR data on a gene-by-gene basis (Table 3) indicated that some method of selection would optimize predictive power, since some of the genes were quite strongly associated with outcome, while others were less so. Table 3 shows results of qRT-PCR analyses on 69 GBM samples. Gene expression levels were determined for each sample for 46 typical survivors (TS) and 23 long-term survivors (LTS). The ratio of the mean expression level in each survival group (fold change) is shown. The direction of survival association (i.e. higher/lower in TS versus LTS) was compared to that found in the microarray data. Genes are sorted in the table first by concordance with microarray data, and then by degree of difference between survival groups. Table 3 shows results of qRT-PCR analyses on 69 exemplary GBM samples.

| | fold change | concordant with |
|---|---|---|
| Gene name | (TS/LTS) | microarray data |
| PDPN | 4.32 | yes |
| AQP1 | 2.94 | yes |
| CHI3L1 | 2.72 | yes |
| RTN1 | 0.37 | yes |
| KIAA0510 | 0.40 | yes |
| GPNMB | 2.05 | yes |
| EMP3 | 2.03 | yes |
| S100A10 | 2.03 | yes |
| IGFBP2 | 1.99 | yes |
| LGALS3 | 1.90 | yes |
| OLIG2 | 0.53 | yes |
| SERPA3 | 1.86 | yes |
| TNC | 1.78 | yes |
| NNMT | 1.76 | yes |
| VEGFA | 1.72 | yes |
| GABBR1 | 0.60 | yes |
| TCTE1L | 1.54 | yes |
| MAOB | 1.53 | yes |
| TAGLN2 | 1.47 | yes |
| TGFBI | 1.41 | yes |
| SERPG1 | 1.38 | yes |
| OMG | 0.74 | yes |
| LGALS1 | 1.36 | yes |
| CLIC1 | 1.33 | yes |
| TIMP1 | 1.32 | yes |
| ACTN1 | 1.31 | yes |
| FABP5 | 1.26 | yes |
| RIS1 | 1.20 | yes |
| LDHA | 1.16 | yes |
| TAGLN | 1.15 | yes |
| TCF12 | 0.88 | yes |
| SERPE1 | 1.10 | yes |
| GRIA2 | 0.92 | yes |
| COL1A2 | 0.95 | no |
| IGFBP3 | 0.95 | no |
| FN1 | 0.94 | no |
| TMSB10 | 0.93 | no |
| PLP2 | 0.66 | no |

In Table 3, gene expression levels were determined for each sample for 46 typical survivors (TS) and 23 long-term survivors (LTS). The ratio of the mean expression level in each survival group (fold change) is shown. The direction of survival association (i.e. higher/lower in TS versus LTS) was compared to that found in the microarray data. Genes are sorted in the table first by concordance with microarray data, and then by degree of difference between survival groups.

Results of the qRT-PCR data on a gene-by-gene basis are shown in Table 4. A systematic approach towards choosing among the genes was chosen. Thirty-three of the 38 genes showed differential expression between TS and LTS in the expected direction. The other five genes (shown at the bottom of Table 3) were excluded from further analysis.

A logistic regression model was used to construct a classifier based on 33 genes for the 69 independent GBM samples. The corresponding binomial log-likelihood was minimized by gradient boosting with component-wise least squares as base learner (Buhlmann et al., 2003). The stratified bootstrap (stratified for TS and LTS) was applied to determine the optimal number of boosting iterations (160 in this case). Six of 33 gene assays were used in this classifier; namely

f=0.0609×(RTN1-0.4773)−

0.1231×(PDPN−2.7583)−

0.0151×(AQP1−3.6225)−

0.0239×(GPNMB−1.321)−

0.0020×(S100A10−2.989)−

0.0204×(IGFBP2−1.3473)

where the prediction is TS when f>0 and LTS for f<0. The computations were performed using the add-on package mboost (Hothorn and Buhlmann et al., 2007).

This model was compared with a random forest classifier with respect to misclassification error and variables selected. The misclassification error for the logistic regression model was about 29% (estimated via stratified bootstrap) whereas 27% misclassification error occurred for the random forest model (out-of-bag error). The variable importance measures for the genes selected by logistic regression are highly ranked among the variable importance for all 38 genes. The package randomForest was used for this analysis (Breiman et al., 2006). This comparison shows that a simple linear formula is appropriate for classification of typical vs. long-term survivors and that the important genes used by both methods coincide. The finding that these six genes are the most informative for prognosis in this dataset should be considered only as an example of the process of optimization of the multigene predictor, and further experiments may be employed to validate an optimal gene set, which may or may not include all or some of the six genes referred to in Example 1, in specific embodiments.

### Example 2

### Statistical Method and Concordance of Survival Association Across Institutions

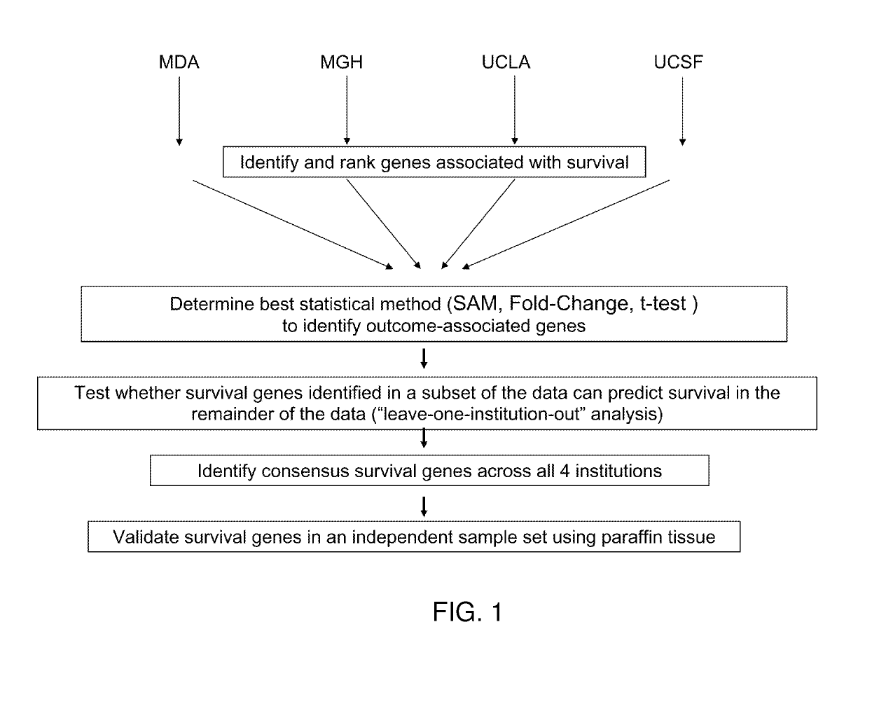

FIG. 1 shows the overall approach utilized for the identification of robust survival-associated genes in GBM. It is not well established which test statistic is optimal to identifying genes significantly associated with patient outcome from microarray data for the purpose of determining consensus genes across independent datasets (Shi et al., 2006). It was thus investigated whether fold-change (the ratio of the means in gene expression measurements between TS and LTS) or SAM performed better in the dataset for identifying common survival-associated genes across multiple institutions. Consistent with recent results from the Microarray Quality Control (MAQC) Project (Shi et al., 2006), the analyses demonstrated that the ranking of genes by degree of fold-change between TS and LTS was much more stable across independent datasets than if genes were ranked by a 2-class SAM analysis (FIG. 5). Fold-change was therefore utilized for subsequent analyses.

### Example 3

### Gene Expression Profiles Predict Survival in Independent Samples of GBM

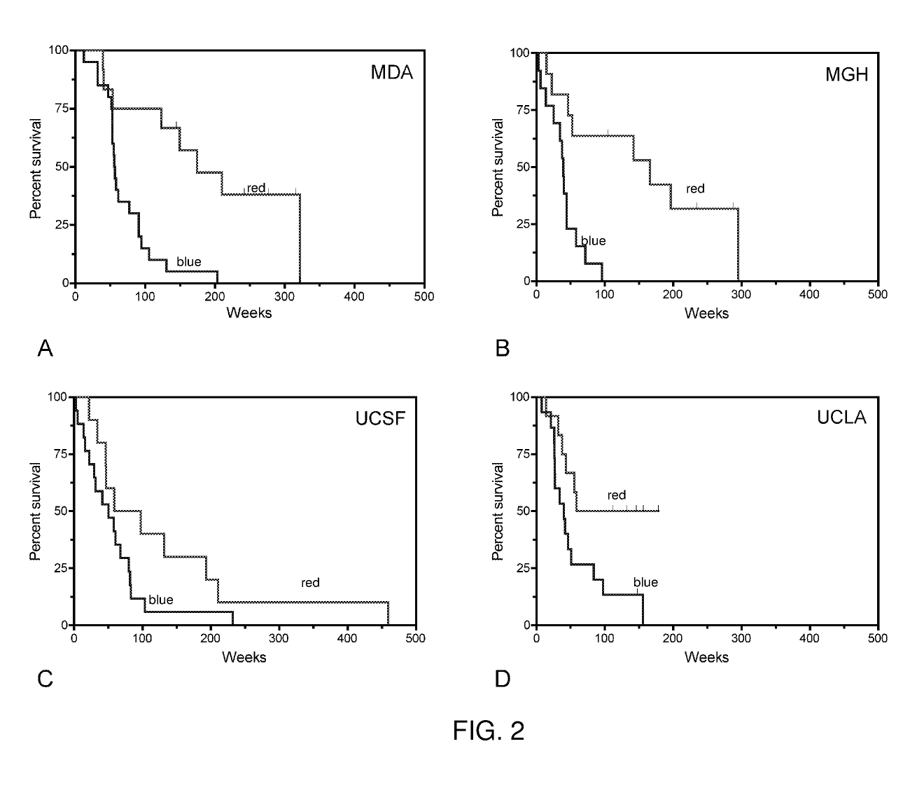

It was tested whether gene expression profiles from one set of GBM tumor samples could predict survival in an independent dataset using a “leave-one-institution-out” approach to cross validation. In each round of the analysis, 3 out of the 4 institutions were utilized to form a training set to identify the top genes associated with survival. The genes were ranked by fold-change difference of TS versus LTS and the top 200 were selected. The performance of this 200-gene profile was then tested for outcome prediction using K-means clustering (Stupp et al., 2005) in the remaining test set (which was not used to build the model). The 2 groups defined by the K-means clustering on the test set were then compared for patient outcome. This procedure was repeated for all (n=4) possible combinations of the datasets. The results (FIG. 2) demonstrated that the survival-associated gene expression profile from the training set showed at least a statistical trend towards survival association in all 4 situations. These data provided proof-of-principle that an outcome-associated gene expression profile obtained from one set of GBM samples could predict survival in an independent dataset. Identification of a consensus multigene predictor of outcome in GBM was then determined.

### Example 4

### Identification of a Consensus Multigene Predictor Across Independent Datasets

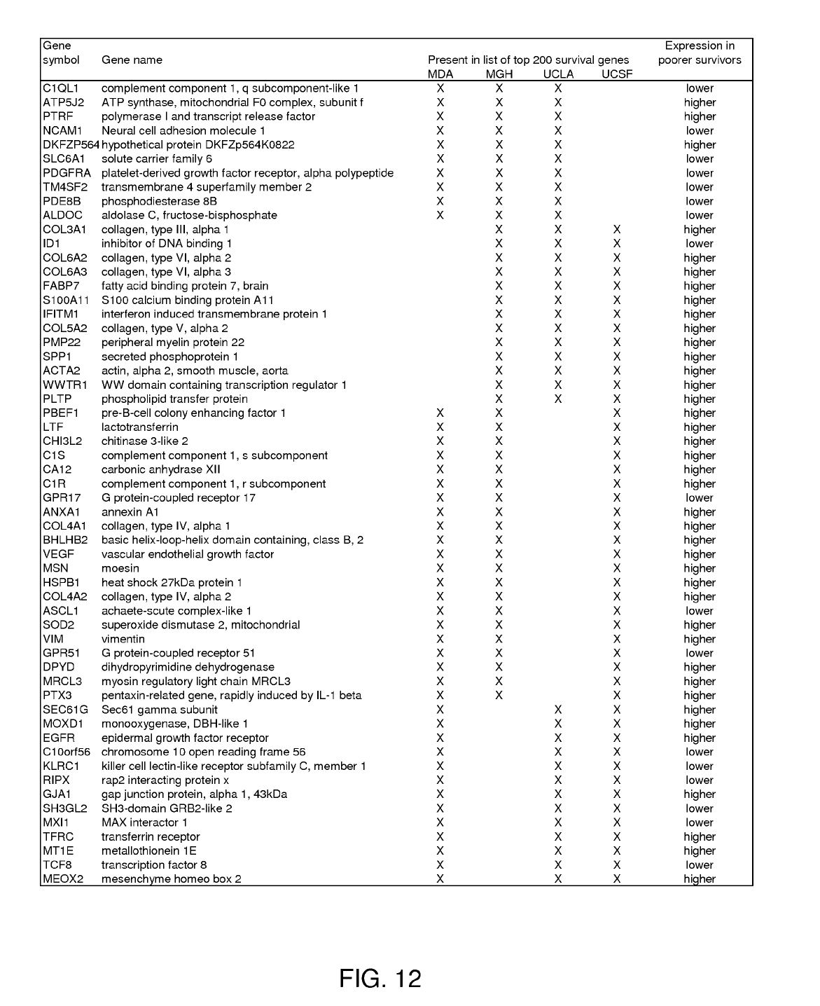

It was then reasoned that the most robust survival genes in GBM would be highly associated with outcome in all 4 datasets. To determine the overlapping survival genes across all 4 institutions, genes were ranked by absolute fold change (TS versus LTS) within each institution, and the common genes ranked in the top 200 genes across all institutions were identified. The results of this analysis are displayed as a Venn diagram in FIG. 3. There were 38 genes (FIG. 3A and Table 4) that were ranked in the top 200 in all 4 institutions, and an additional 57 genes (FIG. 3A and FIG. 12) that were ranked in the top 200 in 3 out of 4 institutions.

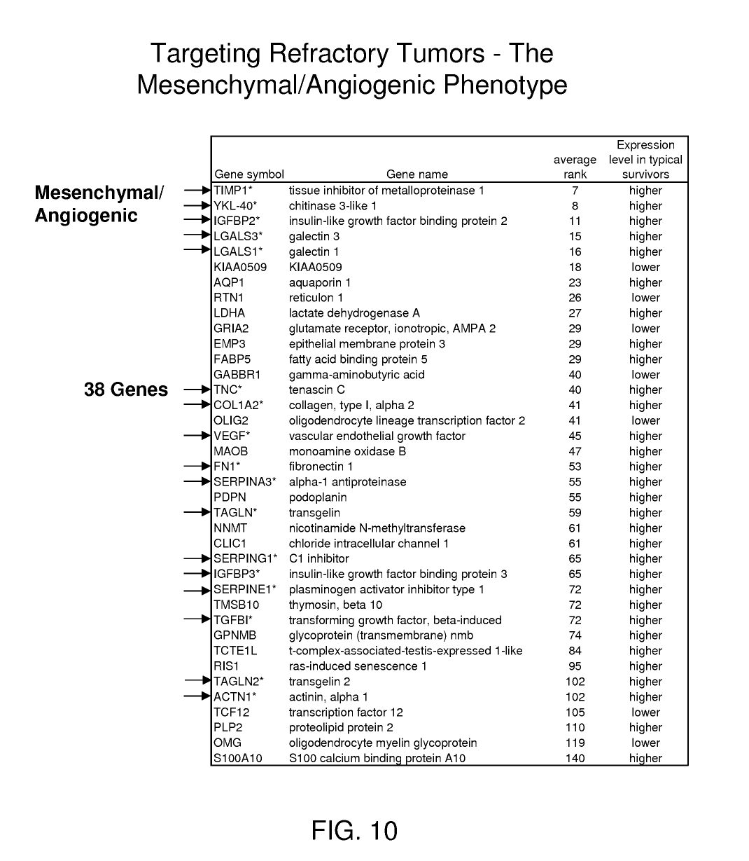

Table 4 shows exemplary survival-associated genes (n=38) common to all 4 microarray datasets. The average fold-change rank between typical and long-term survivors among all 4 microarray datasets is indicated, along with the direction of the association to survival. Genes associated with extracellular matrix/mesnchyme/invasion/angiogenesis are shown with an asterisk. Furthermore, FIG. 10 illustrates 38 genes associated with survival and that are delineated by mesenchymal/angiogenic characterization vs. proneural characterization.

TABLE 4
Exemplary Survival-Associated Genes

| | | | | Expression |
|---|---|---|---|---|
| | | SEQ ID | average | level in typical |
| Gene symbol | Gene name | NO | rank | survivors |
| TIMP1* | tissue inhibitor of metalloproteinase 1 | 1 | 7 | higher |
| YKL-40* | chitinase 3-like 1 | 2 | 8 | higher |
| IGFBP2* | insulin-like growth factor binding protein 2 | 3 | 11 | higher |
| LGALS3* | galectin 3 | 4 | 15 | higher |
| LGALS1* | galectin 1 | 5 | 16 | higher |
| KIAA0509 | KIAA0509 | 6 | 18 | lower |
| AQP1 | aquaporin 1 | 7 | 23 | higher |
| RTN1 | reticulon 1 | 8 | 26 | lower |
| LDHA | lactate dehydrogenase A | 9 | 27 | higher |
| GRIA2 | glutamate receptor, ionotropic, AMPA 2 | 10 | 29 | lower |
| EMP3 | epithelial membrane protein 3 | 11 | 29 | higher |
| FABP5 | fatty acid binding protein 5 | 12 | 29 | higher |
| GABBR1 | gamma-aminobutyric acid | 13 | 40 | lower |
| TNC* | tenascin C | 14 | 40 | higher |
| COL1A2* | collagen, type I, alpha 2 | 15 | 41 | higher |
| OLIG2 | oligodendrocyte lineage transcription factor 2 | 16 | 41 | lower |
| VEGF* | vascular endothelial growth factor | 17 | 45 | higher |
| MAOB | monoamine oxidase B | 18 | 47 | higher |
| FN1* | fibronectin 1 | 19 | 53 | higher |
| SERPINA3* | alpha-1 antiproteinase | 20 | 55 | higher |
| PDPN | podoplanin | 21 | 55 | higher |
| TAGLN* | transgelin | 22 | 59 | higher |
| NNMT | nicotinamide N-methyltransferase | 23 | 61 | higher |
| CLIC1 | chloride intracellular channel 1 | 24 | 61 | higher |
| SERPING1* | C1 inhibitor | 25 | 65 | higher |
| IGFBP3* | insulin-like growth factor binding protein 3 | 26 | 65 | higher |
| SERPINE1* | plasminogen activator inhibitor type 1 | 27 | 72 | higher |
| TMSB10 | thymosin, beta 10 | 28 | 72 | higher |
| TGFBI* | transforming growth factor, beta-induced | 29 | 72 | higher |
| GPNMB | glycoprotein (transmembrane) nmb | 30 | 74 | higher |
| TCTE1L | t-complex-associated-testis-expressed 1-like | 31 | 84 | higher |
| RIS1 | ras-induced senescence 1 | 32 | 95 | higher |
| TAGLN2* | transgelin 2 | 33 | 102 | higher |
| ACTN1* | actinin, alpha 1 | 34 | 102 | higher |
| TCF12 | transcription factor 12 | 35 | 105 | lower |
| PLP2 | proteolipid protein 2 | 36 | 110 | higher |
| OMG | oligodendrocyte myelin glycoprotein | 37 | 119 | lower |
| S100A10 | S100 calcium bindina protein A10 | 38 | 140 | higher |

Expression of 31 of the 38 most robust survival genes was higher in TS compared with LTS, while the remaining 7 had higher expression in LTS. As shown in FIG. 3B the identification of a set of 38 genes associated with survival common to all 4 institutional datasets was highly unlikely to have occurred by chance. The calculated false discovery rates for the identification of genes common to 4 out of 4 datasets using this approach is a 0.3% chance to find 1 common gene among the four lists by chance, and a 99.7% chance that 0 genes would be common to the 4 lists by chance. Among the 31 poor-prognosis genes, many (n=17) of them are associated with mesenchymal differentiation, extracellular matrix or angiogenesis (e.g. LAGALS1, FN1, VEGF). The 7 good-prognosis genes are preferentially associated with neural development (e.g. OLIG2, RTN1, TNR).

In order to determine the association of this gene expression classifier with patient outcome, the 38-gene signature was used to calculate a single “metagene” score for each case. Each tumor was then ranked according to this metagene score. The rankings were condensed into quartiles and the resulting Kaplan Meier survival curves of these 4 groups (FIG. 3C) show a significant association of metagene score with survival, particularly for the group in the lowest quarter (best survival). In order to assess the relationship of gene expression with the prediction of therapeutic efficacy, radiation response was examined. The metagene score was also found to be significantly associated with radiation response in the subset of cases for which imaging studies were available (FIG. 3D). Overall, these data indicate that this 38-gene set represents a consensus profile predictive of outcome across 4 independent datasets from different institutions, and provides a set of candidate genes to test in additional tumor samples.

Since the prior studies indicated that favorable-prognosis GBM's have an expression profile similar to lower grade gliomas (Phillips et al., 2006), it was reasoned that a robust set of survival-associated genes in GBM should overlap with genes found to be differentially expressed between GBM and lower grade gliomas. This embodiment was characterized in an independent published dataset of 153 glioma tumor samples of different grades (Sun et al., 2006) using the data analysis tool from Oncomine (see Oncomine website). Comparing the top 2% of genes overexpressed in GBM versus lower grade gliomas in that dataset with the 38-gene set, it was found that 26 of the 31 poor-prognosis genes were concordant. These results provided independent confirmation that the consensus gene list is likely to be a robust predictor of outcome in GBM.

### Example 5

### Validation of Multigene Predictor of Survival and Radiation Response

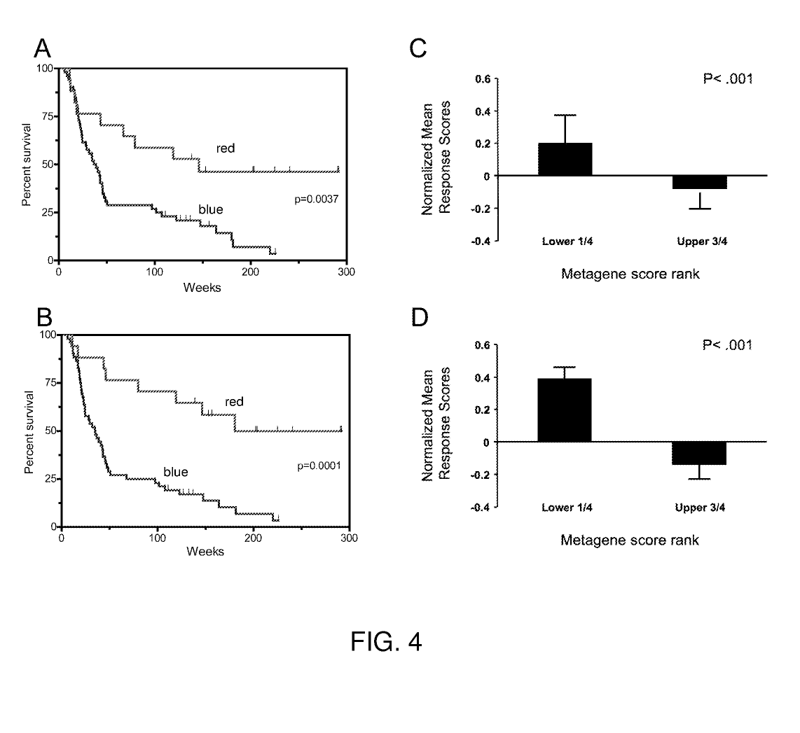

To perform initial validation of the 38-gene predictor, an independent retrospective set of FFPE tumor samples of 69 newly diagnosed GBMs were utilized, none of which were used in the prior microarray analyses. Utilizing qRT-PCR assays optimized for measurement of gene expression from FFPE tissue, the expression of each of the 38 genes was quantified in the 69 GBM samples. Expression of each individual gene was normalized to the average expression of two control genes (GAPDH and GUSB) and the fold-change difference between survival groups is summarized for each gene assay in Table 3. For each case, a metagene score was calculated using the method similar to that used for the microarray data. As seen in the microarray data, samples in the lowest quarter of metagene scores have significantly better survival compared to samples in the upper 3 quarters (p=0.0037, log rank test) when the scores were calculated from the entire 38-gene set (FIG. 4A). The association of 38-gene metagene score and radiation response was also significant, validating the microarray data (FIG. 4C).

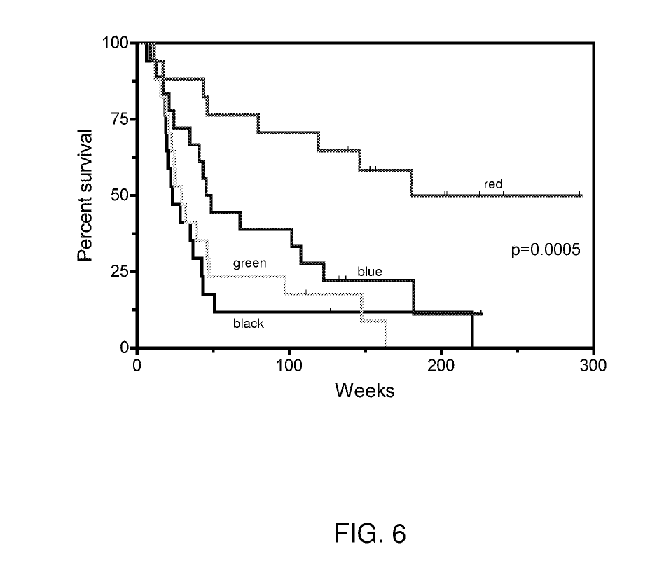

There was further optimization of the genes to be assayed with qt-PCR in the multigene predictor for future applications and identification of those genes that contribute most to survival prediction from the larger set of 38 genes. To explore this, a logistic regression model was constructed with implicit variable selection and shrinkage fitted by a gradient boosting algorithm with componentwise least squares (Buhlmann et al., 2003). Six genes resulted from this analysis (PDPN, AQP1, GPNMB, S100A10, IGFBP2, RTN1) and the model resulted in a slight improvement in outcome prediction compared to the unweighted metagene model. Bootstrapping cross-validation (×100) of the linear predictor was performed and indicated that the model was particularly good at correctly classifying the 43 TS patients, since a mean value of 35 (81%) TS patients were correctly classified in cross-validation. An alternative classifier was constructed using a second statistical approach, random forest classification (Breiman, 2001; Breiman et al., 2006). Random forest classification identified the same 6 genes with nearly identical classification rates. Ranking tumor samples by a metagene score based on these 6 genes and comparing the lowest quarter to the remaining samples demonstrated an increased association with both survival (FIG. 4B) and radiation response (FIG. 4D). The Kaplan-Meier curves for all 4 quarters based on the 6-gene score are shown in FIG. 6). A receiver operating characteristic curve fitted for the prediction of 2-year survival based on the linear classifier gave an area under the curve (AUC) of 0.788 (95% CI 0.667-0.910), which compared favorably to an AUC fitted for patient age (0.687, 95% CI 0.548-0.830), the most powerful known predictor of outcome in GBM.

### Example 6

### Molecularly Guided Study in Glioblastoma

Recent advances have improved standard treatment for GBM patients, with temozolomide chemoradiation (TMZ-CR) significantly improving median survival (Stupp et al., 2005). However, it is clear that only a fraction of patients derive significant benefit from this treatment, with overall two-year survival in the TMZ-CR treated patients in this study only reaching 26%. These findings are consistent with longstanding clinical and recent molecular evidence that subtypes of GBM exist with differing survival rates and response to treatment, but the diagnosis and treatment decisions in GBM are currently based on histopathology alone.

To move towards individualization/optimization of treatment in GBM, it is useful to: 1) develop sensitive and specific markers to prospectively distinguish those patients who will respond to standard therapy from those who will not respond; and 2) Identify important molecular alterations in tumors to guide optimization of therapy in the next generation of hypothesis-driven trials with agents targeted at patients with specific molecular profiles.

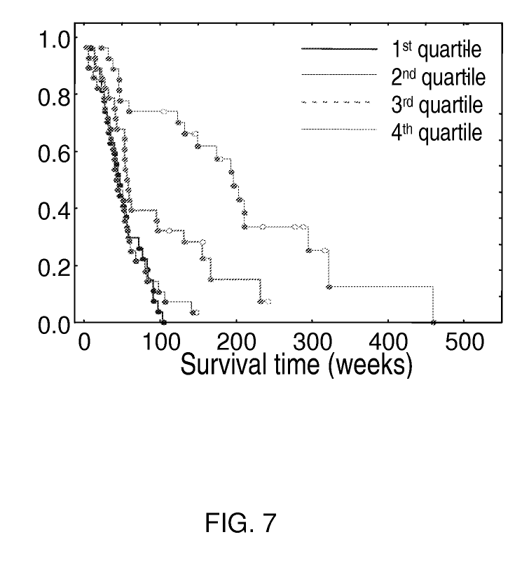

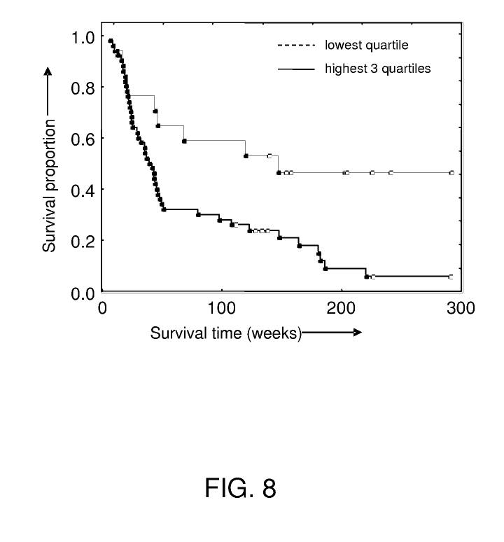

Toward this end, the inventors have conducted a meta-analysis of gene expression microarray data from multiple institutions and identified a 38-gene set that is a robust predictor of 2-year survival in independent data sets (FIGS. 3A, 3B, and 7). Initial evaluation of a subset of the 38 genes using quantitative RT-PCR (QRT-PCR) from formalin-fixed paraffin-embedded (FFPE) samples from an independent set of 68 newly diagnosed GBMs (FIG. 8) indicates that this gene expression panel is a robust predictor of outcome to treatment with radiation therapy and alkylating agents. Furthermore, these studies demonstrate the feasibility of utilizing a panel of QRT-PCR based assays for prospective optimization of treatment for individual GBM patients from FFPE tissue, as has been successfully implemented in breast cancer (Paik et al., 2004).

Analysis of this 38-gene set, along with prior studies from the inventors (Nigro et al., 2005; Phillips et al., 2006), demonstrate that overexpression of genes associated with mesenchymal transition and angiogenesis is associated with poor prognosis and treatment resistance. These data indicate that a neuro-epithelial to mesenchymal transition occurs in GBM, as has been observed in a number of epithelial cancers, and is associated with poor outcome and resistance to standard therapy. Furthermore, data from the inventors and others also demonstrates that activation of the PI3-K/AKT/mTOR and MAPK pathways are associated with worse outcome and resistance to therapy in GBM (Nigro et al., 2005; Haas-Kogan et al., 2005; Mellinghoff et al., 2005; Pelloski et al., 2006).

The invention, in specific embodiments, concerns the following: 1) that GBMs can be prospectively classified into clinically distinct treatment groups based on a a robust multi-marker predictor; and 2) that small molecule inhibitors of the ras/raf, VEGFR, and AKT/mTOR pathways will target the mesenchymal/angiogenic phenotype in GBM and provide a therapeutic benefit to patients resistant to standard therapy.

In general embodiments of the present invention, there is optimization and characterization of a multi-marker panel for prediction of patient outcome (time to progression) in newly diagnosed GBM patients treated with standard therapy. In specific embodiments, there is development and optimization of the multimarker set using QRT-PCR assays for the 38 genes in FFPE tissue, IHC markers for activation of the AKT/MAPK pathway, and MGMT promoter methylation for prediction of patient outcome in a retrospective set (n=68) of UTMDACC GBM cases. Statistical modeling is used to define a multi-marker panel integrating significant predictive markers.

In specific embodiments, there is validation of the multi-marker predictor panel in an independent set of GBM samples from patients treated with temozolomide chemoradiation (n=100) from UT MD Anderson. In further specific embodiments, the inventors will leverage the resources of collaboration in the NCI TCGA project to identify novel markers of patient outcome utilizing gene expression, array CGH, and epigenetic profiling of matched frozen tissue samples from tumors.

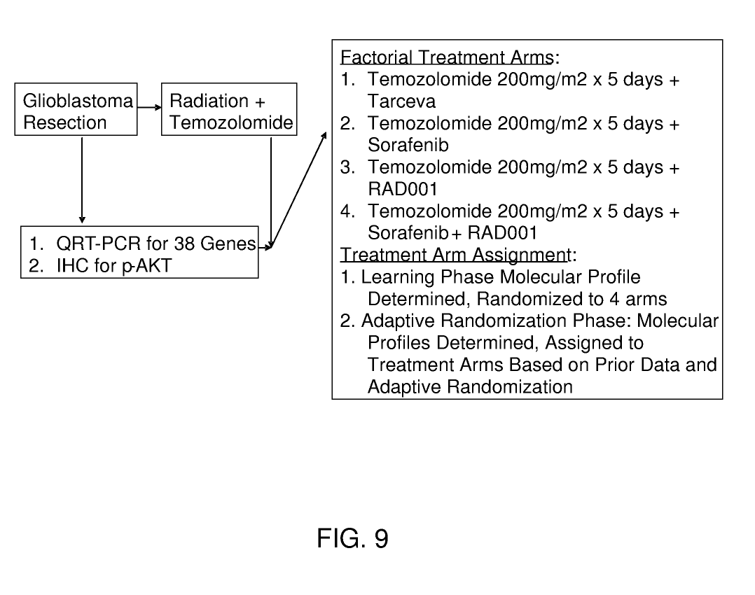

In another general embodiment, the inventors conduct a prospective phase I/II study utilizing the multi-marker panel to optimize individual patient treatment in newly diagnosed GBM (FIG. 9). In specific embodiments, the inventors demonstrate the feasibility of utilizing the 38-gene set and AKT pathway status from paraffin-embedded samples for prospective treatment decision making in newly diagnosed GBM. In further specific embodiments, the inventors test the hypothesis that treatment with TMZ-CR and inhibition of the AKT/mTOR pathway with RAD001 and/or inhibition of the raf/VEGFR pathways with Sorafenib will improve progression-free survival in poor prognosis GBM patients with the mesenchymal/angiogenic phenotype compared to historical controls. In additional specific embodiments, the inventors will leverage the resources of the role as the source of brain tumor samples for the NCI TCGA project to identify novel biomarkers predictive of response to the small molecule inhibitors RAD001 and Sorafenib in molecular sub-groups of patients.

### Methodology and Study Design

Optimization and Validation of Molecular Markers: Tissue resources: the inventors will utilize retrospectively collected samples from MDACC, with appropriate clinical annotation and follow-up. Archival paraffin blocks are available for all of these patients and the majority will also have frozen tissue available. QRT-PCR: Paraffin tissues will be selected for the QRT-PCR assay using macrodissection (based on a representative H&E) to ensure purity of tumor. RNA is isolated and extracted using methods optimized in the labs. cDNA is made using random hexamer priming. Primers and probes optimized for QRT-PCR in FFPE tissue are optimized by designing primers and probes with inter-primer distances less than 75 bp. All gene assays as well as 3 control genes (GAPDH, GUSB, ACTB) will be performed in triplicate. Outlier values will be excluded. DeltaCt values will be calculated based on the average Ct values for each gene relative to the average Ct of the four control genes. AKT/MAPK activation and MGMT promoter methylation: IHC will be performed at MDACC using standard/established methods. The detection and scoring using phospho-specific antibodies for AKT and MAPK may be employed. Scoring will be semi-quantitative based on a combination of staining intensity and number of cells stained. IHC for phospho-specific markers may be employed, and the inventors have shown in several to be associated with outcome in GBM (Pelloski et al., 2006). The methylation status of MGMT will be assessed using bisulfite treatment/methyl specific-PCR as previously described (Hegi et al., 2005). Statistical considerations: Time to progression may be used as the endpoint, unless a patient dies without radiographic evidence of progression, in which case time to death will be used. In specific aspects, the present inventors may assess classifier performance by using the area under the Receiver Operating Characteristic curve. The IHC data may be incorporated into the expression data as well as MGMT status. These additional markers are added to the set of genes selected as described above and the analyses repeated. This will allow the inventors to assess how much the new markers add to the predictive accuracy of the model and the relative ordering of the various markers. The inventors may perform diagonal linear discriminant analysis (DLDA) and choose the DLDA model with the smallest number of top markers that yields appropriate prediction error. This model may then be validated using an independent dataset of patients treated with TMZ-CR.

### Prospective Trial Design in Newly Diagnosed GBM

Patient Inclusion: All patients will have undergone biopsy or resection for newly diagnosed GBM, and FFPE blocks must be available for analysis. Study Design: All patients will receive standard external beam radiation therapy combined with temozolomide at 75 mg/m² daily. Molecular analysis including QRT-PCR, MC, and MGMT promoter methylation will be performed for each patient during the 6-week radiation treatment period. A factorial study design will be utilized (FIG. 9). Based on the current data, in specific embodiments, good prognosis patients patients (good prognosis multigene score and low p-AKT) will have a high likelihood of durable response to radiation and temozolomide, and an increased likelihood of response to an EGFR inhibitor. Thus, one treatment arm will consist of adjuvant temozolomide at 200 mg/m² on a 5 out of 28 day schedule+Tarceva. Based on the gene expression and IHC data, in specific embodiments, patients with a poor prognosis multigene score and/or high p-AKT are unlikely to have durable survival with standard therapy alone or addition of an EGFR inhibitor. Thus, three of the factorial arms will be designed to improve progression-free survival in this group and will consist of combination therapy targeted at the mesenchymal/angiogenic phenotype. These three arms will include temozolomide (200 mg/m² on a 5 out of 28 day schedule), with the additional therapy for each arm consisting of: 1) Sorafenib, 2) RAD001, 3) Sorafenib+RAD001. Molecular Profile and Treatment Assignment: During the initial learning phase of the trial, patients will be randomly assigned to the four treatment arms. Real-time analysis of association between molecular profile and patterns of failure on each arm will be utilized to estimate predictive power for response to individual treatment combinations and test the initial hypotheses related to molecular profile and response to therapy. In the second phase, adaptive randomization will be used based initially on data from the learning phase to prospectively assign patients to specific treatment arms based on molecular profile. Endpoints: Primary Endpoint=Time to progression. Secondary Endpoints=2 year survival, radiographic response, molecular correlates of response and survival (see below). Statistical Considerations: Comparison will be made to historical controls with appropriate molecular data based on a multigene model. While calculation of exact sample size will depend on analysis of these historical controls, in specific embodiments, a sample size of about 68 patients in each of the poor prognosis treatment groups will provide sufficient statistical power. Thus, there will be a total of 120 total patients that receive either drug (Sorafenib or RAD001), and 60 patients that will receive the combination. So, this design provides increased power to determine potential efficacy of each agent, and will also allow correlation of molecular sub-types with response to each agent individually and in combination. Additional Correlative Studies: Comprehensive molecular analyses will be performed at the DNA (CGH), RNA (Expression Profiling), and epigenetic levels on frozen tissue available from these patients through both the Kleburg Center, and involvement with NCI Cancer Genome Atlas Project (TCGA) initiative. Specifically widespread profiling (DNA/RNA/epigenetic) of a large number of tumor samples from a limited number of tumor types is planned through the NCI TCGA. GBM was selected as one of the tumor types and M. D. Anderson was selected as the tissue repository which will supply the GBM samples. The end result will be a large (several hundred) set of clinically annotated samples on which CGH, expression profiling and promoter methylation data are available. Most of the samples in the current proposal will also be profiled as part of the TCGA project, thus adding significant additional data regarding molecular correlates of response and patient outcome to specific therapies. This combined effort will further leverage the observations from the current proposal and contribute significantly to the discovery of novel clinically relevant marker combinations in GBM. Protein lysate arrays and additional high-throughput molecular screens will be performed through the Kleburg Center at MDACC. Results of these analyses will be correlated with the primary and secondary endpoints to identify novel markers of treatment response to these individual agents. Due to the ability of the invention design to incorporate new molecular predictor data in real-time, the present invention provides the ability to rapidly incorporate novel robust molecular predictors identified during the discovery phase of the studies.

### Example 7

### Determination of Glioblastoma Prognosis and/or Therapy Response

In particular aspects of the invention, an individual is assayed for glioblastoma prognosis and/or therapy response by determining the level of RNA transcripts, or expression products thereof, for each of one or more genes listed in Table 4. In particular cases, the expression level for each genes is normalized, for example to the expression level of a housekeeping gene or to the expression level of all RNA transcripts. Then, a single “metagene” score is calculated for an individual based on the set of 38 genes in Table 4 by summing the normalized expression values for all the genes associated with poor prognosis and then subtracting the sum of the normalized expression values for all the genes associated with good prognosis for the individual. This results in a single numerical score for each tumor, a tumor value, and each tumor is then ranked according to this value (which may be referred to as a metagene score).

The tumor value is compared to the values found in a reference glioblastoma tissue set, wherein a collective expression level in about the upper 75th percentile indicates an increased risk of poor prognosis and/or poor response to radiation-chemotherapy and a collective expression level in about the lower 25th percentile indicates an increased chance of good prognosis and/or good response to radiation-chemotherapy.

### Example 8

### 38 Exemplary Genes Associated with Survival

Glioblastoma (GBM) is the most common and aggressive primary brain tumor. There are currently no molecular diagnostic markers in routine clinical use. In a meta-analysis of microarray data sets, a consensus 38 gene set was identified that was significantly associated with patient outcome in all the data sets. The 38-gene signature was tested on an independent set of 69 GBM paraffin embedded tumor samples. Both the full 38-gene set and an optimized 14-gene subset demonstrated a highly significant association with both survival and radiographic response to radiation therapy. The optimized 14-gene set was tested in a separate set of 77 GBM tumors from uniformly treated patients who all received the standard therapy, and was shown to be a powerful predictor of outcome.

Final validation of the optimized multigene predictor is being carried out in the current Phase III study, RTOG 0525, which will enroll over 1100 patients. The validated predictor aids in optimization of therapy in newly diagnosed GBM by distinguishing those individuals who will experience durable survival from standard therapy alone versus those individuals for whom standard therapy will be of little or no benefit, and who will be better served by more aggressive therapy or clinical trials targeting the mesenchymal/angiogenic phenotype.

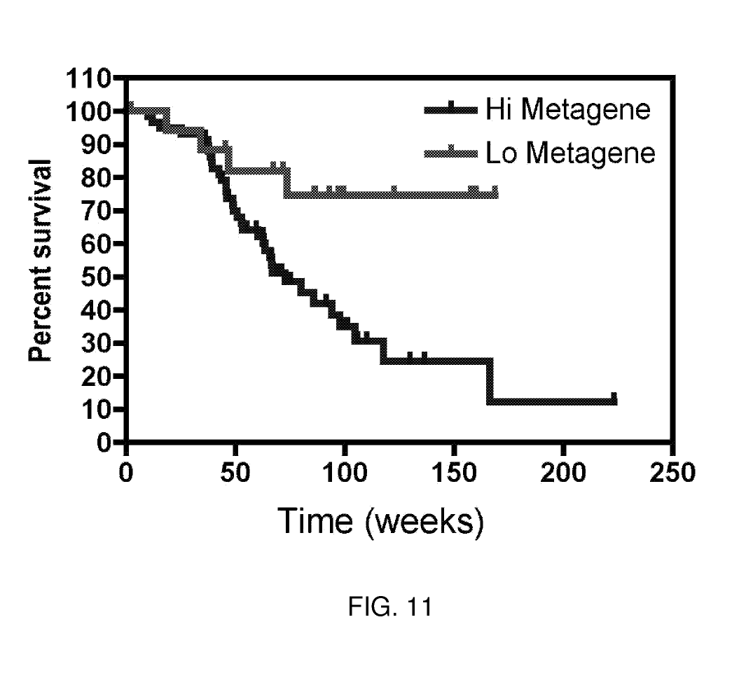

Table 4 and FIG. 10 provide 38 exemplary genes associated with survival, including their fold expression change. Calculation of metagene score from these illustrative 38 genes includes the “bad” gene expression average minus the “good” gene expression average. In specific embodiments, high metagene score is associated with worse outcome. FIG. 11 demonstrates that metagene score is associated with survival and radiographic response.

In some embodiments of the invention, there is clinical application of the multigene predictor. In particular, there is a clinical assay for predicting outcome to standard therapy in GBM. In particular cases, the test is amenable to routinely processed, clinically available tissue, for example formalin-fixed, paraffin-embedded specimens. Validation of an independent set is employed (for example, Oncotype Dx assay for breast cancer (Genomic Health)). In specific examples for validation of multigene predictor, multiple GBM samples are tested and may comprise isolation of RNA from samples, such as paraffin blocks. The expression level of the 38 genes and control genes (for example, 4 control genes) is measured using quantitative RT-PCR. Primer/probes may be optimized for fragmented RNA, for example. An exemplary enterprimer distance is less than about 75 bases.

### Example 9

### Validation of an Exemplary Gene Predictor in Radiation-Treated GBM

Validation of an exemplary gene predictor in radiation-treated GBM was investigated For example, FIG. 11 illustrates validation of exemplary 14-Gene Predictor in temozolomide-radiation treated GBM.

Clinical application of a multigene predictor is employed. Validation in RTOG 0525 (n=1100 patients, paraffin block mandatory). Additional optimization in retrospective samples are employed, in specific embodiment. QRT-PCR assays may be adapted to a higher-throughput analysis platform. One may be able to utilize a molecular profile to optimize therapy, in some embodiments, for example, utilizing molecular stratification and/or prospective determination of optimal therapy for individual patients.

In specific embodiments, refractory tumors exhibit mesenchymal/angiogenic phenotype, and this is targeted in GBM. For example, in newly diagnosed GBM, the multigene predictor is utilized. When a favorable molecular profile is identified, the individual may be administered TMZ/radiation. When an unfavorable molecular profile is identified, the individual may be administered TMZ/radiation plus an alternative therapy, including anti EMT and/or an antiangiogenic agent, for example.

### Example 10

### Significance of the Embodiments of the Present Invention

Currently, treatment of newly diagnosed GBM is relatively uniform despite variation in response to standard therapy. To identify markers of outcome, the present invention identifies a consensus multigene panel to distinguish patients with favorable versus unfavorable survival. Given the strong correlation of treatment response and survival in GBM28, such a marker panel is utilized not only for prognostic purposes, but also to aid in the prospective identification of likelihood of response to standard treatment, in certain embodiments of the invention. A meta-analysis of Affymetrix data was performed from 4 separate institutions. Examination of several statistical approaches for analysis of survival-associated genes demonstrated that use of fold change (using mean expression measurements between typical and long-term survivors) resulted in the highest concordance across institutions, consistent with previous inter-institutional meta-analyses of microarray data (Shi et al., 2006). A prognostic model can successfully pass cross validation tests with a leave-one-institution-out approach. By determining the top prognostic genes common to all 4 of the individual institution data, a multigene set associated with patient survival as well as radiation response is identified, a measure previously shown to be tightly linked with survival in GBM (Barker et al., 1996). Utilizing qRT-PCR assays optimized for measurement of gene expression from FFPE tissue, this multigene set is validated as a predictor of both survival and radiation response. Cross-validation using the top 6 genes from the multigene predictor identified with the logistic regression model demonstrated the robustness of this gene sub-set for outcome prediction from qRT-PCR data. Together, these findings demonstrate the feasibility of developing a clinically applicable gene expression classifier for individualization of patient treatment in GBM.

Practical considerations drove the choice to utilize FFPE tissues as a means of validation. Identification of biomarkers amenable to use in FFPE tissue allows broader clinical application in patient samples for which frozen tissue specimens are unavailable and are unlikely to become available (e.g. samples from multi-institutional/cooperative group clinical trials). In addition, the future incorporation of additional candidate markers of treatment response in GBM (Haas-Kogen et al., 2005; Mellinghoff et al., 2005; Chakravarti et al., 2004; Pelloski et al., 2005; Pelloski et al., 2006) in this multigene predictor improves robustness for prospective treatment assignment of the individual patient, in certain aspects of the invention. Linear regression and random forest analyses identified a 6-gene predictor from the qRT-PCR data. This 6-gene set provides an example of refinement of the gene set for survival prediction.

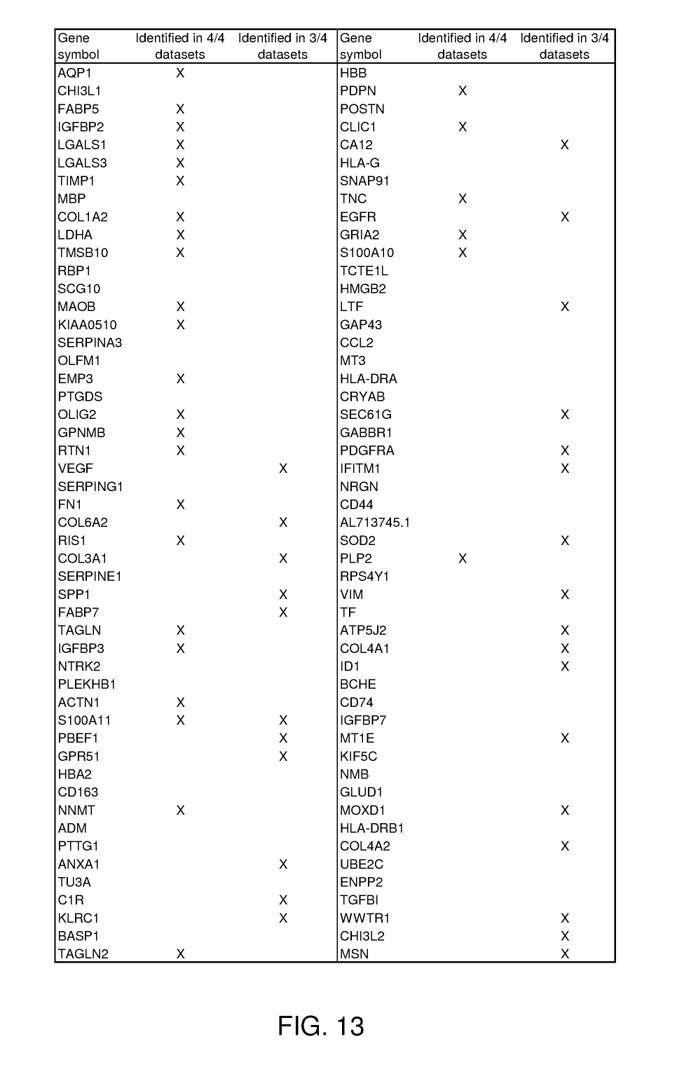

The use of fold-change (ratio of average gene expression levels between survival groups) as a method to identify concordant outcome-associated genes in microarray studies has been suggested as superior to methods based on t-statistic p-values (Shi et al., 2006), and this was found to be the case when applied to the data in this meta-analysis. The Rank Product method has been recently suggested to be a promising means to detect consistent gene expression differences in replicated microarray experiments (Breitling et al., 2005; Breitling et al., 2004) and fold-change is a key component of the Rank Product. Application of the Rank Product method to the microarray data showed an excellent concordance of survival-associated genes with the 38-gene set (FIG. 13).

Taken together, the results and those of others (Shi et al., 2006) indicate that the degree of difference (i.e. fold change) of gene expression among groups of samples is an important measure for the identification of robust biomarkers from microarray data.

In addition to its role as a predictive/prognostic tool, the identification of a multigene set with robust association with outcome provides potential insights into tumor biology that can have therapeutic implications. Functional analysis of the 38 genes demonstrates that better prognosis is associated with higher expression of genes associated with normal neural development, while poor survival is associated with increased expression of genes associated with mesenchymal tissues, angiogenesis, and extracellular matrix. Immunohistochemical analyses have demonstrated that a number of these mesenchymal and angiogenic genes including YKL-40 (Pelloski et al., 2005), galectin-1, galectin-3, tenascin (Leins et al., 2003; McLendon et al., 2000), VEGF (Ding et al., 2001), are indeed expressed by GBM tumor cells (as opposed to non-neoplastic cells). Prior unsupervised (i.e. without regard for survival) analyses by the inventors and others (Freije et al., 2004; Phillips et al., 2006; Tso et al., 2006) have identified similar genes as markers of distinct molecular subtypes of GBM. The current study extends these findings by demonstrating that similar genes and functional groups are also prominent in a directed search for the most robust survival-associated markers. Taken together, these data indicate that a clinically relevant mesenchymal transition occurs in GBM that is associated with poor outcome and is analogous to the epithelial-to-mesenchymal transition that has been described in carcinomas (Thiery et al., 2000). The mesenchymal/angiogenic gene expression pattern profile is therefore useful both as a molecular stratification, and as new therapeutic targets for individuals who will not respond to conventional therapy, in particular aspects of the invention.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XII. References

The following references, to the extent that they provide exemplary procedural or other details supplementary to those set forth herein, are specifically incorporated herein by reference:

### PATENTS AND PATENT APPLICATIONS

U.S. Pat. No. 5,705,629
U.S. Pat. No. 4,458,066
U.S. Pat. No. 4,659,774
U.S. Pat. No. 4,816,571
U.S. Pat. No. 5,141,813
U.S. Pat. No. 5,264,566
U.S. Pat. No. 4,959,463
U.S. Pat. No. 5,427,916
U.S. Pat. No. 5,428,148
U.S. Pat. No. 5,554,744
U.S. Pat. No. 5,574,146
U.S. Pat. No. 5,602,244
U.S. Pat. No. 4,683,202
U.S. Pat. No. 4,682,195
U.S. Pat. No. 5,645,897,

### PUBLICATIONS

Barker F G, 2nd, Prados M D, Chang S M, et al. Radiation response and survival time in patients with glioblastoma multiforme. J Neurosurg 1996; 84(3):442-8.
Breiman L, Cutler A, Liaw A, Wiener M. randomForest: Breiman and Cutler's Random Forests for Classification and Regression. In; 2006.
Breiman L. Random Forests. Machine Learning 2001; 24:123-40.
Breitling R, Armengaud P, Amtmann A, Herzyk P. Rank products: a simple, yet powerful, new method to detect differentially regulated genes in replicated microarray experiments. FEBS Lett 2004; 573(1-3):83-92.
Breitling R, Herzyk P. Rank-based methods as a non-parametric alternative of the T-statistic for the analysis of biological microarray data. J Bioinform Comput Biol 2005; 3(5):1171-89.
Bühlmann P, Yu B. Boosting with L2 Loss: Regression and Classification. Journal of the American Statistical Association 2003; 98(462):324-38.
Burton E C, Lamborn K R, Feuerstein B G, et al. Genetic aberrations defined by comparative genomic hybridization distinguish long-term from typical survivors of glioblastoma. Cancer Res 2002; 62(21):6205-10.
Camby I, Belot N, Rorive S, et al. Galectins are differentially expressed in supratentorial pilocytic astrocytomas, astrocytomas, anaplastic astrocytomas and glioblastomas, and significantly modulate tumor astrocyte migration. Brain Pathol 2001; 11(1):12-26.
Chakravarti A, Zhai G, Suzuki Y, et al. The prognostic significance of phosphatidylinositol 3-kinase pathway activation in human gliomas. J Clin Oncol 2004; 22(10):1926-33.
Ding H, Roncari L, Wu X, et al. Expression and hypoxic regulation of angiopoietins in human astrocytomas. Neuro-oncol 2001; 3(1):1-10.
Fan C, Oh D S, Wessels L, et al. Concordance among gene-expression-based predictors for breast cancer. N Engl J Med 2006; 355(6):560-9.
Freije W A, Castro-Vargas F E, Fang Z, et al. Gene expression profiling of gliomas strongly predicts survival. Cancer Res 2004; 64(18):6503-10.
Haas-Kogan D A, Prados M D, Lamborn K R, Tihan T, Berger M S, Stokoe D. Biomarkers to predict response to epidermal growth factor receptor inhibitors. Cell Cycle 2005; 4(10):1369-72.
Hegi M E, Diserens A C, Gorlia T, et al. MGMT gene silencing and benefit from temozolomide in glioblastoma. N Engl J Med 2005; 352(10):997-1003.
Imanishi T, Itoh T, Suzuki Y, et al. Integrative annotation of 21,037 human genes validated by full-length cDNA clones. PLoS Biol 2004; 2(6):e162.
Kleihues P, Cavenee W, eds. WHO Classification of Tumours: Pathology and Genetics of Tumours of the Nervous System. Lyon: IARC Press; 2000.
Leins A, Riva P, Lindstedt R, Davidoff M S, Mehraein P, Weis S. Expression of tenascin-C in various human brain tumors and its relevance for survival in patients with astrocytoma. Cancer 2003; 98(11):2430-9.
Liang Y, Diehn M, Watson N, et al. Gene expression profiling reveals molecularly and clinically distinct subtypes of glioblastoma multiforme. Proc Natl Acad Sci USA 2005; 102(16):5814-9.
McLendon R E, Wikstrand C J, Matthews M R, Al-Baradei R, Bigner S H, Bigner D D. Glioma-associated antigen expression in oligodendroglial neoplasms. Tenascin and epidermal growth factor receptor. J Histochem Cytochem 2000; 48(8):1103-10.
Mellinghoff I K, Wang M Y, Vivanco I, et al. Molecular determinants of the response of glioblastomas to EGFR kinase inhibitors. N Engl J Med 2005; 353(19):2012-24.
Nigro J M, Misra A, Zhang L, et al. Integrated array-comparative genomic hybridization and expression array profiles identify clinically relevant molecular subtypes of glioblastoma. Cancer Res 2005; 65(5):1678-86.
Nutt C L, Mani D R, Betensky R A, et al. Gene expression-based classification of malignant gliomas correlates better with survival than histological classification. Cancer Res 2003; 63(7):1602-7.
Paik S, Shak S, Tang G, et al. A multigene assay to predict recurrence of tamoxifen-treated, node-negative breast cancer. N Engl J Med 2004; 351(27):2817-26.
Pelloski C E, Lin E, Zhang L, et al. Prognostic associations of activated mitogen-activated protein kinase and Akt pathways in glioblastoma. Clin Cancer Res 2006; 12(13):3935-41.
Pelloski C E, Mahajan A, Maor M, et al. YKL-40 expression is associated with poorer response to radiation and shorter overall survival in glioblastoma. Clin Cancer Res 2005; 11(9):3326-34.
Phillips H S, Kharbanda S, Chen R, et al. Molecular subclasses of high-grade glioma predict prognosis, delineate a pattern of disease progression, and resemble stages in neurogenesis. Cancer Cell 2006; 9(3):157-73.
Potti A, Mukherjee S, Petersen R, et al. A genomic strategy to refine prognosis in early-stage non-small-cell lung cancer. N Engl J Med 2006; 355(6):570-80.
Pruitt K D, Tatusova T, Maglott D R. NCBI Reference Sequence project: update and current status. Nucleic Acids Res 2003; 31(1):34-7.
Ransohoff D F. Rules of evidence for cancer molecular-marker discovery and validation. Nat Rev Cancer 2004; 4(4):309-14.
Rich J N, Hans C, Jones B, et al. Gene expression profiling and genetic markers in glioblastoma survival. Cancer Res 2005; 65(10):4051-8.
Shi L, Reid L H, Jones W D, et al. The MicroArray Quality Control (MAQC) project shows inter- and intraplatform reproducibility of gene expression measurements. Nat Biotechnol 2006; 24(9):1151-61.
Simon R. Roadmap for developing and validating therapeutically relevant genomic classifiers. J Clin Oncol 2005; 23(29):7332-41.
Stupp R, Mason W P, van den Bent M J, et al. Radiotherapy plus concomitant and adjuvant temozolomide for glioblastoma. N Engl J Med 2005; 352(10):987-96.
Sun L, Hui A M, Su Q, et al. Neuronal and glioma-derived stem cell factor induces angiogenesis within the brain. Cancer Cell 2006; 9(4):287-300.
Thiery J P. Epithelial-mesenchymal transitions in tumour progression. Nat Rev Cancer 2002; 2(6):442-54.
Tso C L, Shintaku P, Chen J, et al. Primary glioblastomas express mesenchymal stem-like properties. Mol Cancer Res 2006; 4(9):607-19.
Tusher V G, Tibshirani R, Chu G. Significance analysis of microarrays applied to the ionizing radiation response. Proc Natl Acad Sci USA 2001; 98(9):5116-21.
Zhang L, Miles M F, Aldape K D. A model of molecular interactions on short oligonucleotide microarrays. Nat Biotechnol 2003; 21(7):818-21

1. A method of screening an individual for glioblastoma prognosis and/or response to glioblastoma therapy, comprising assessing the expression levels of the RNA transcripts of the genes listed in Table 4, or their protein translation products, in a glioblastoma cell sample from the individual, as normalized in relation to the expression levels of one or more reference RNA transcripts, or their protein translation products, and determining a prognosis or therapeutic response by means of said comparison.

2. The method of claim 1, wherein increased expression, as compared to the reference RNA transcripts, of one or more of KIAA0509, RTN1, GRIA1, GABBR1, OLIG2, TCF12, C10orf56, ID1, PDGFRA, C1QL1 and OMG indicates a favorable prognosis and/or favorable response to therapy, and/or wherein increased expression, as compared to the reference RNA transcripts, of one or more of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10, PBEF, LTF1, CHI3L2, SEC61G, DKFZp564K0822, and EGFR, indicates an unfavorable prognosis and/or unfavorable response to therapy.

3. The method of claim 1, further defined as:
(a) determining the expression levels of RNA transcripts from two or more genes listed in Table 4;
(b) normalizing the expression levels of the RNA transcripts from two or more genes to expression levels of one or more reference RNA transcripts;
(c) subtracting the sum of the normalized expression values for the RNA transcripts from genes associated with favorable prognosis and/or therapy response from the sum of the normalized expression values for the RNA transcripts from genes associated with unfavorable prognosis and/or therapy response, wherein said subtracting results in a tumor value;
(d) comparing the tumor value with reference glioblastoma tumor values, wherein a tumor value that is in the upper 75^{th} percentile relative to the reference glioblastoma tumor values indicates an unfavorable prognosis and/or therapy response and wherein a tumor value that is in the lower 25^{th} percentile relative to the reference glioblastoma tumor values indicates a favorable prognosis and/or therapy response,
wherein the genes associated with favorable prognosis and/or therapy response are selected from the group consisting of KIAA0509, RTN1, GRIA1, GABBR1, OLIG2, TCF12, C10orf56, ID1, PDGFRA, C1QL1 and OMG, and
wherein the genes associated with unfavorable prognosis and/or therapy response are selected from the group consisting of TIMP1, YKL-40, IGFBP2, LGALS3, LGALS1, AQP1, LDHA, EMP3, FABP5, TNC, COL1A2, VEGF, MAOB, FN1, SERPINA3, PDPN, TAGLN, NNMT, CLIC1, SERPING1, IGFBP3, SERPINE1, TMSB10, TGFB1, GPNMB, TCTE1L, RIS1, TAGLN2, ACTN1, PLP2, S100A10, PBEF, LTF1, CHI3L2, SEC61G, DKFZp564K0822, and EGFR.

4. (canceled)

5. (canceled)

6. (canceled)

7. The method of claim 1, wherein the method is screening an individual for glioblastoma prognosis.

8. The method of claim 1, wherein the method is screening an individual for response to glioblastoma therapy.

9. The method of claim 1, wherein the one or more reference RNA transcripts are further defined as RNA transcripts of one or more housekeeping genes.

10. The method of claim 9, wherein the housekeeping genes are selected from the group consisting of glyceraldehyde-3-phosphate-dehydrogenase (GAPDH), β-glucuronidase, actin, ubiquitin, albumin, cytochrome, and tubulin.

11. The method of claim 1, wherein the glioblastoma therapy comprises radiation, chemotherapy, or a combination thereof.

12. The method of claim 11, wherein the chemotherapy is further defined as comprising one or more alkylating agents.

13. The method of claim 11, wherein the chemotherapy comprises temozolomide, carmustine, cyclophosphamide, procarbazine, lomustine, and vincristine, carboplatin, irinotecan, erlotinib, sorafenib, RAD001, or a combination thereof.

14. The method of claim 1, wherein said assessing comprises polymerase chain reaction, microarray analysis, or immunoassay.

15. A kit comprising an isolated collection of nucleic acids that hybridize under stringent conditions to the RNA transcripts from at least 5, 10, 15, 20, 25, 30, or 35 of the genes listed in Table 4.

16. (canceled)

17. (canceled)

18. (canceled)

19. (canceled)

20. (canceled)

21. (canceled)

22. The kit of claim 15, wherein the nucleic acids hybridize under stringent conditions to RNA transcripts from at least five of the genes selected from the group consisting of PDPN, AQP1, YKL40, GPNMB, EMP3, S100, IGFBP2, LGALS3, SERPE3, TNC, NNMT, VEGFA, TCTEIL, MAOB, TAGLN2, RTN1, KIAA0510, OLIG2, GABA, EGFR, CHI3L2, C1QL1, PDGFRA, ID1, and LTF.

23. The kit of claim 15, further comprising nucleic acids that hybridize under stringent conditions to RNA transcripts from fifteen or fewer, twelve or fewer, ten or fewer, seven or fewer, five or fewer, or two or fewer housekeeping genes.

24. (canceled)

25. (canceled)

26. (canceled)

27. (canceled)

28. (canceled)

29. The kit of claim 23, wherein the housekeeping genes are selected from the group consisting of glyceraldehyde-3-phosphate-dehydrogenase (GAPDH), β-glucuronidase, actin, ubiquitin, albumin, cytochrome, and tubulin.

30. The kit of claim 15, wherein the isolated collection of nucleic acids are housed on a substrate.

31. The kit of claim 35, wherein the substrate is a microarray chip.

32. A collection of oligonucleotides, wherein each of said oligonucleotides hybridizes under stringent conditions to an RNA transcript from a gene listed in Table 4.

33. The collection of claim 32, wherein the oligonucleotides are further defined as primers for polymerase chain reaction.

34. The collection of claim 33, wherein the collection comprises two or more primers for an RNA transcript from each of at least two, five, ten, fifteen, twenty, twenty-five, thirty, or thirty-five genes listed in Table 4.

35. (canceled)

36. (canceled)

37. (canceled)

38. (canceled)

39. (canceled)

40. (canceled)

41. (canceled)

42. The collection of claim 33, wherein the collection comprises three or more primers for an RNA transcript from each of at least two, five, ten, fifteen, twenty, twenty-five, thirty, or thirty-five genes listed in Table 4.

43. (canceled)

44. (canceled)

45. (canceled)

46. (canceled)

47. (canceled)

48. (canceled)

49. (canceled)
